(19) United States
(12) Patent Application Publication
(10) Pub. No.: US 2025/0002983 A1
(43) Pub. Date: Jan. 2, 2025

(54) MASSIVELY PARALLEL CHARACTERIZATIONS OF MULTIPLE TARGET TYPES IN MULTI-DIMENSIONAL SPACE

(71) Applicant: Curio Bioscience, Inc., Palo Alto, CA (US)

(72) Inventors: Stephen P.A. FODOR, Palo Alto, CA (US); Hei Mun Christina FAN, Palo Alto, CA (US); Christina CHANG, San Mateo, CA (US); Julie WILHELMY, Santa Cruz, CA (US); Wanxin WANG, Mountain View, CA (US)

(21) Appl. No.: 18/752,298

(22) Filed: Jun. 24, 2024

Related U.S. Application Data
(63) Continuation of application No. PCT/US24/34537, filed on Jun. 18, 2024.
(60) Provisional application No. 63/523,600, filed on Jun. 27, 2023.

(51) Int. Cl. C12Q 1/6841

(52) U.S. Cl. CPC C12Q 1/6841

(57) ABSTRACT

Systems, methods, and compositions for generating a high-resolution spatial map of a distribution of targets of a sample are described. Systems and methods can further include features configured to reduce observation of smearing artifacts in generated spatial maps. Processes for generating the spatial map can include: receiving the sample at a substrate having a distribution of functionalized particles, each having a stochastic barcode sequence paired with a position on the substrate; promoting interactions between the distribution of targets of the sample and the distribution of functionalized particles; applying a set of reactions to the sample at the substrate; and obtaining a set of sequences of a population of molecules generated from the set of reactions.

### CROSS-REFERENCE

This application is a continuation of International Application No. PCT/US24/34537, filed Jun. 18, 2024, which claims the benefit of U.S. Provisional Application No. 63/523,600, filed Jun. 27, 2023, which are incorporated in their entirety herein by these references.

### STATEMENT AS TO FEDERALLY SPONSORED RESEARCH

The invention(s) disclosed herein was made with Government support under 5R44HG012532 awarded by the National Human Genome Research Institutes. The Government has certain rights in the invention(s).

### TECHNICAL FIELD

This invention relates generally to the sample characterization field, and more specifically to new and useful systems, methods, and compositions for characterizing locations of target analytes.

### BACKGROUND

With an increased interest in understanding distributions of particular target analytes within a biological sample, improved compositions, methods, and systems that allow for analyte mapping are becoming highly valuable. Current technologies are limited in resolution (e.g., with respect to location of target analytes), ability to characterize locations in multiple dimensions, ability to characterize locations across scales of magnitude, ability to characterize different types of analytes, ability to characterize locations of targets in situ, and/or in other manners. Furthermore, compositions for enabling mapping can require high precision and uniformity in composition in order to enable accurate characterization of target locations in space. Thus, there is a need in the sample characterization field for new and useful systems, methods, and compositions for characterizing locations of target analytes.

### SUMMARY OF THE INVENTION

Currently, methods and systems for spatially resolving single or multiple analytes in space (e.g., in situ, in vitro, etc.) are limited in relation to: resolution (e.g., with respect to potential number of target analytes that can be characterized per unit area or volume, with respect to scale of position determination for target analytes), low signal to noise ratio (e.g., due to high levels of background noise), empty/unused space between individual sites for target analytes interactions, ability to characterize locations in multiple dimensions, ability to characterize locations across scales of magnitude, ability to characterize different types of analytes, ability to characterize single cell subtypes without referencing single cell databases (e.g., using spatial marker genes alone), ability to characterize locations of targets in situ, ability to characterize locations of targets in vitro, and/or in other manners.

Accordingly, this disclosure describes embodiments, variations, and examples of systems, methods, and compositions for performing spatial biology (e.g., spatial transcriptomics, spatial proteomics, spatial multi-omics, etc.), in a manner that provides broader transcriptome coverage while achieving high levels of spatial resolution, with significant reductions in levels of mapping artifacts.

An aspect of the disclosure provides embodiments, variations, and examples of systems, methods, and compositions for efficient capture and labeling of target material (e.g., DNA, RNA, miRNA, proteins, small molecules, single analytes, multianalytes, etc.) to enable analyses for characterizing locations of target material in space. For nucleic acid targets, capture probes of compositions described can include complementary molecules to the nucleic acid targets. For protein targets or small molecule targets, capture probes of the compositions described can include antibodies or aptamers conjugated with specific nucleic acid sequences for detection.

Targets can include cytoplasmic targets, intracellular targets, or other targets on the surface of or within a cell (or components of a cell). For instance, targets can include cytoplasmic targets and targets of or associated with nuclei of cells from the same tissue, such that simultaneous mapping of multiple target types in a highly parallel manner can be achieved. In one embodiment, spatial labeling of nuclei of a sample can include: processing a sample comprising a set of nuclei with a substrate comprising a distribution of functionalized particles (e.g., with decoding of positions of the functionalized particles by way of stochastic/spatial barcodes, as described below); tagging the set of nuclei with the stochastic/spatial barcodes (e.g., upon cleaving functionalized molecules from the functionalized particles); isolating nuclei of the set of nuclei and/or other targets of the sample; and determining positions of nuclei of the set of nuclei upon sequencing molecules generated from the distribution of functionalized particles. Optionally, in some embodiments, methods can include capture (e.g., microfluidic capture, capture within microwells, capture within partitions, capture within droplets of an emulsion) and barcoding of nuclei targets (e.g., mRNAs, other nuclei targets) and spatial barcodes after isolation of nuclei. Identification of nuclei targets can further optionally be performed using optical detection of nuclei targets tagged with probes during capture and barcoding of nuclei, without sequencing.

In embodiments, a substrate can include functionalized particles including a first subset of functionalized particles for tagging nuclei targets of a sample, and a second subset of functionalized particles for tagging cytoplasmic targets of the sample. In embodiments, a substrate can include functionalized particles for tagging only nuclei targets (e.g., nuclear RNA) of a sample. In embodiments, a substrate can include functionalized particles for tagging only cytoplasmic targets (e.g., cytoplasmic RNA) of a sample. In embodiments a substrate can be functionalized with functionalized particles to tag whole single cells of a sample.

In relation to tissue processing, the disclosure provides methods, systems, and devices for spatially labeling targets (e.g., cytoplasmic targets) that are exposed during tissue processing (e.g., tissue slicing, tissue sectioning), as well as spatially labeling of individual nuclei and/or other intracellular components. Methods described can include determining locations of cell bodies (e.g., based upon nuclei positions) of a sample, and performing single-cell analysis techniques in coordination with spatial analysis of single-cell and other targets, where analysis techniques can include single nucleus RNA-seq (snRNA-seq), t-cell receptor (TCR) analyses, b-cell receptor (BCR) analyses (e.g., with receptor-ligand characterizations), ATAC-seq for assessment of chromatin accessibility, processing of nuclei and non-nuclei targets of formalin-fixed and paraffin-embedded (FFPE) samples, analysis of nuclear DNA, analysis of nuclear proteins, and/or other analyses.

Aspects of the disclosure also provide embodiments, variations, and examples of systems, methods, and devices for accurately determining positions (e.g., relative positions, positions on a substrate having functionalized particles, positions relative to another reference point or surface, etc.) of nuclei of a sample. In embodiments where a nucleus is tagged using cleavable molecules of functionalized particles, tagging of the nucleus can involve tagging the nucleus with different cleavable molecules associated with different spatial positions. As such, determining the position of the nucleus can involve determining the position based upon a subset of positions corresponding to a subset of stochastic barcodes of molecules that tagged the nucleus. The position of the nucleus can be determined from an average position of the subset of stochastic barcodes (e.g., a centroid of positions of the subset of stochastic barcodes). In variations, nuclei can be tagged using a combination of cleavable and non-cleavable molecules, such that positions of the nuclei can be determined from stochastic/spatial barcode positions of cleavable and non-cleavable molecules (e.g., as a weighted centroid of positions, where positions of non-cleavable components are weighted more heavily than positions of cleavable components).

Aspects of the disclosure also provide embodiments, variations, and examples of systems, methods, and devices that solve cell segmentation issues and sensitivity issues associated with mapping cell-associated targets (e.g., cytoplasmic targets, nuclei-associated targets, etc.). The systems, methods, and devices solve cell segmentation issues and sensitivity issues associated with mapping of targets in human tissue.

Aspects of the disclosure also provide embodiments, variations, and examples of systems, methods, and compositions for generating spatial maps of a set of targets of a sample, in a manner that provides high performance with respect to reducing an amount of artifacts present in resultant target maps. Reducing artifacts (e.g., smearing artifacts) in resultant target maps can be achieved using sample processing and/or bioinformatics approaches, as described in more detail below. In particular, with respect to sample processing steps, application of a layer (e.g., layer of OCT compound, described in more detail below) can result in a percent reduction of smearing and background artifacts in generated maps, where in examples, the percent reduction in smearing was: greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, or greater. In examples, the percent reduction in smearing was determined by comparing values (e.g., percentage values, number values) of functionalized particles to which noise (e.g., noise associated with target movement and re-capture) was attributed, between a first section of a tissue sample and a second section of the tissue sample, where the smear-prevention layer was added to the first section, and was not added to the second section. In examples, the percent reduction in smearing was determined by comparing values (e.g., percentage values, number values) of UMIs/bead detected during sequencing, to which noise (e.g., noise associated with target movement and re-capture) was attributed, between a first section of a tissue sample and a second section of the tissue sample, where the smear-prevention layer was added to the first section, and was not added to the second section.

With respect to bioinformatics approaches, such approaches can result in a percent reduction of smearing and background artifacts in generated maps, where in examples, the percent reduction in smearing was: greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 95%, or 100%. In examples, the percent reduction in smearing was determined by comparing values (e.g., percentage values, number values) of functionalized particles to which noise (e.g., noise associated with target movement and re-capture) was attributed, between a first spatial map of targets generated without application of described bioinformatics processes, and a second spatial map of targets generated with application of described bioinformatics processes. In examples, the percent reduction in smearing was determined by comparing values (e.g., percentage values, number values) of UMIs/bead detected during sequencing, to which noise (e.g., noise associated with target movement and re-capture) was attributed, between a first spatial map of targets generated without application of described bioinformatics processes, and a second spatial map of targets generated with application of described bioinformatics processes.

Functionalized features (e.g., spots, particles, other features described) on the substrate can have a center-to-center spacing less than 20 micrometers, less than 19 micrometers, less than 18 micrometers, less than 17 micrometers, less than 16 micrometers, less than 15 micrometers, less than 14 micrometers, less than 13 micrometers, less than 12 micrometers, less than 11 micrometers, less than 10 micrometers, less than 9 micrometers, less than 8 micrometers, less than 7 micrometers, less than 5 micrometers, less than 4 micrometers, less than 3 micrometers, less than 2 micrometers, less than 1 micrometers, or less.

Substrates described can have at least: 50,000 functionalized features, 60,000 functionalized features, 70,000 functionalized features, 80,000 functionalized features, 90,000 functionalized features, 100,000 functionalized features, 120,000 functionalized features, 140,000 functionalized features, 160,000 functionalized features, 180,000 functionalized features, 200,000 functionalized features, 300,000 functionalized features, 400,000 functionalized features, 500,000 functionalized features, 600,000 functionalized features, 700,000 functionalized features, 800,000 functionalized features, 900,000 functionalized features, 1 million functionalized features, 2 million functionalized features, 5 million functionalized features, 10 million functionalized features, 15 million functionalized features, 20 million functionalized features, 25 million functionalized features, 30 million functionalized features, or greater.

Aspects of the disclosure also provide embodiments, variations, and examples of systems, methods, and compositions for generating spatial maps of a set of targets of a sample, where the spatial maps have a resolution of greater than one target mapped per 500 um², greater than one target mapped per 400 um², greater than one target mapped per 300 um², greater than one target mapped per 200 um², greater than one target mapped per 150 um², greater than one target mapped per 100 um², greater than one target mapped per 50 um², greater than one target mapped per 40 um², greater than one target mapped per 30 um², greater than one target mapped per 20 um², greater than one target mapped per 10 um², greater than one target mapped per 9 um², greater than one target mapped per 8 um², greater than one target mapped per 7 um², or any intermediate number of targets mapped per unit area.

Mapping can be performing for each of a set of at least 2 targets, 3 targets, 4 targets, 5 targets, 6 targets, 7 targets, 8 targets, 9 targets, 10 targets, 11 targets, 12 targets, 13 targets, 14 targets, 15 targets, 16 targets, 17 targets, 18 targets, 19 targets, 20 targets, 25 targets, 30 targets, 40 targets, 50 targets, 100 targets, 500 targets, 1000 targets, 5000 targets, 10000 targets, 15000 targets, 20000 targets, 30000 targets, 40000 targets, 50000 targets, 100000 targets, or any intermediate number of targets simultaneously, at resolutions described. Targets can be a single type of target (e.g., cytoplasmic mRNA targets, other target types described) or alternatively, targets can comprise different types of targets that can be captured and mapped using the same unit of the system. As such, mapping can be achieved for characterizations of multiple target types (see example in FIG. 11), in multi-dimensional space, using the same unit of the system (e.g., substrate with functionalized particles).

In relation to capture and mapping of nuclei targets, the disclosure provides embodiments, variations, and examples of systems, methods, and compositions for generating spatial maps with improved recovery rate for nuclei targets (e.g., in relation to actual numbers of nuclei targets present) to greater than 15%, greater than 20%, greater than 25%, greater than 30%, greater than 35%, greater than 40%, greater than 45%, greater than 50%, greater than 60% or greater. In particular, nuclei retention is typically poor due to losses during sample processing, capture of nuclei (e.g., using microfluidic approaches), and isolation of nuclei, and the disclosure provides methods for improved recovery rate and retention of nuclei.

In relation to tagging of nuclei and/or other targets of a sample with functionalized molecules of functionalized particles, functionalized molecules can include unique molecular identifiers (UMIS). The disclosure provides embodiments, variations, and examples of systems, methods, and compositions for recovering greater than 2000 UMIs per nucleus/target, greater than 2500 UMIs per nucleus/target, greater than 3000 UMIs per nucleus/target, greater than 3500 UMIs per nucleus/target, greater than 4000 UMIs per nucleus/target, greater than 4500 UMIs per nucleus/target, greater than 5000 UMIs per nucleus/target, greater than 5500 UMIs per nucleus/target, greater than 6000 UMIs per nucleus/target, greater than 6500 UMIs per nucleus/target, greater than 7000 UMIs per nucleus/target, greater than 7500 UMIs per nucleus/target, greater than 8000 UMIs per nucleus/target, greater than 8500 UMIs per nucleus/target, greater than 9000 UMIs per nucleus/target, greater than 9500 UMIs per nucleus/target, greater than 10,000 UMIs per nucleus/target, greater than 20,00 UMIs per nucleus/target, or greater. As such, systems, methods, and devices described can achieve higher UMI capture per nucleus/target, in relation to existing single-cell techniques and other state-of-the-art techniques.

Generated maps can have an associated signal-to-noise ratio (SNR) of at least 100, 200, 300, 400, 500, 600, 700, 800, 900, 1000, 2000, 3000, 4000, 5000, 6000, 7000, 8000, 9000, 10000, 100000, or greater, where background noise is attributed to leakage of targets away from their original positions at the sample and toward functionalized particles that are positioned further away from the original positions at the sample. As such, SNR can be determined by calculating a ratio between a number of signal copies observed at “correct” positions and a number of background copies of targets present in “background” positions). In variations, determining the SNR can include: identifying one or a set of genes known to be expressed in regions (e.g., single cell types, single cell subtypes) of a sample (where such genes can be expressed at low level, such as less than 100 copies per particle, or at high level); quantifying expression of the one gene or the set of genes across the sample (e.g., according to method steps described below), determining a value of the signal from a measure of the expression of the one gene or the set of genes in regions that should express such gene(s), and determining a value of the noise from a measure of the expression of the one gene or the set of genes in regions that should not express such genes. The SNR can then be determined from the value of the signal divided by the value of the noise. In a specific example, for a mouse hippocampus sample, the SNR was determined using hippocalcin hpca) gene and transthyretin (ttr) gene, which are known to be expressed by certain hippocampus cell subtypes and not represented in other sample regions. Using hpca and ttr, the value of the noise was determined to be 0, indicating that hpca and ttr genes were not observed in regions that should not express hpca/ttr. As such, the SNR for the exemplary sample type was shown to be infinite. Values of signal and noise can be determined from raw or normalized counts. Higher levels of noise can be attributed to anchoring of oligonucleotides onto glass substrates directly (e.g., without a particle layer intermediary), which can create different surface physical characteristics that promote target leakage and thus greater levels of background noise.

Generated maps can have an associated false positive rate less than a threshold number (e.g., a false positive percentage), where the false positive rate is determined from a percent (e.g., x %) of positive copies of targets observed beyond a threshold distance (e.g., y micrometers) away from where the positive copies (“signal”) should actually originate from. In examples, the false positive rate can be less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, or less than another suitable percent. In examples, the threshold distance can be 15 micrometers, 14 micrometers, 13 micrometers, 12 micrometers, 11 micrometers, 10 micrometers, 9 micrometers, 8 micrometers, 7 micrometers, 6 micrometers, 5 micrometers, 4 micrometers, 3 micrometers, 2 micrometers, or another suitable threshold distance, where background noise is attributed to leakage of targets away from their original positions at the sample and toward functionalized particles that are positioned further away from the original positions at the sample.

Aspects of the disclosure also provide embodiments, variations, and examples of systems for generating spatial maps of a set of targets of a sample, where the empty/unused space between substrate features (e.g., beads or other particle bodies, rods, protrusions, recesses, ridges, valleys, channels, wells, oligonucleotide spots, etc.) for generating such spatial maps is less than 45 micrometers, 40 micrometers, 35 micrometers, 30 micrometers, 25 micrometers, 20 micrometers, 15 micrometers, 10 micrometers, 9 micrometers, 8 micrometers, 7 micrometers, 6 micrometers, 5 micrometers, 5 micrometers, 4 micrometers, 3 micrometers, 2 micrometers, 1 micrometer, 0.5 micrometers, 0.25 micrometers, 0.1 micrometers, or intermediate distances.

Aspects of the disclosure also provide embodiments, variations, and examples of systems, methods, and compositions for generating spatial maps of a set of targets of a sample, where the spatial maps have a resolution of less than a threshold distance between features (e.g., beads or other particle bodies, rods, protrusions, recesses, ridges, valleys, channels, wells, oligonucleotide spots, etc.) of a substrate for target capture/interactions. Embodiments, variations, and examples of spatial maps generated have a resolution of less than 50 picometers between features, less than 40 picometers between features, less than 30 picometers between features, less than 20 picometers between features, less than 10 picometers between features, less than 5 picometers between features, or less than 1 picometer between features.

In particular, in relation to platforms involving non-close-packed features (e.g., functionalized particles) for target capture and mapping, the invention(s) described achieve high resolution mapping with minimal (or non-existent noise, as described above), by having smaller functional unit areas and spatial unit areas for target capture. Furthermore, the systems described minimize the amount of empty or dead space between features for target capture, by close packing such features (e.g., in comparison to platforms where features are printed as spots that are spaced apart at the surface of a substrate, such as a glass slide). In examples, as shown in FIG. 1C, structural configurations of the features for target mapping, as in the inventions described, produce smaller capture areas per feature (and therefore higher resolution data), smaller spatial unit areas, smaller functional unit areas, less leakage of targets from the sample and therefore lower levels of background noise, and the ability to identify features (e.g., single cell subtypes) based upon a clustering analysis of spatial biomarkers, and without requiring deconvolution or other more involved computational approaches.

Aspects of the disclosure provide embodiments, variations, and examples of systems, methods, and compositions for spatially characterizing samples in multidimensions (e.g., 2D, 3D, 4D with a time component), in relation to one or more of: whole tissue structures, tissue pieces (e.g., as in histology, in relation to biopsied tissues, in relation to seeded natural scaffolds, in relation to seeded synthetic scaffolds (e.g., cell-seeded hydrogel scaffolds, cell-seeded polaxamer scaffolds, etc.) in relation to frozen tissue specimens (e.g., fresh frozen tissue samples that are sectioned), in relation to formalin-fixed and paraffin-embedded (FFPE) specimens, fresh frozen plasma etc.), frozen cell suspensions, cell suspensions retained in a medium/hydrogel medium organs, whole organisms, organoids, cell suspensions, single cells, organelles, sub-organelle structures, intra-organelle components, mitochondrial targets, viruses, microorganisms, and other natural structures. Cells can include mammalian cells, bacteria, microbes, plant cells, fungal cells, or other cells/cell-like components.

Location characterization can additionally or alternatively be performed in relation to non-naturally occurring structures, such as microwells, microarrays, scaffolds, and other non-naturally occurring structures. For instance, the invention(s) can have in situ and/or in vivo applications, with infusion of functionalized particles into a sample (e.g., into a cell, into a tissue, into an organ, etc.). Examples of infusion can include one or more of: injection, electroporation, use of vectors (e.g., viral vectors), and other infusion methods.

In relation to single cell characterizations associated with various tissue types or other sample types, aspects of the disclosure provide embodiments, variations, and examples of methods for generating a spatial map of single cell subtypes of the sample based upon spatial biomarkers alone (e.g., without referencing single cell reference databases). In particular, given particle sizes implemented in the invention(s) described, single cell subtypes can be determined using unsupervised clustering architecture, without requiring deconvolution (e.g., which is used when the functional area for target capture is larger (e.g., larger than 20 microns)). Examples of the methods described are able to achieve identification of microglia, endothelial cells, astrocyte subtypes, interneurons, neurons, oligodendrocytes, polydendrocytes, entorihinal, ependymal, choroid, neurogenesis, cajal retzius, mural, and other tissue cell subtypes by applying a clustering analysis to marker genes alone.

In relation to mapping of different target types using the same unit of the system for capturing multiple target types, the invention(s) can achieve mapping of single cell types/subtypes of a sample along with mapping of distributions of nuclei targets, cytoplasmic targets, and/or other targets, from the same sample and using the same unit of the system (e.g., distribution of functionalized particles coupled to a substrate).

Aspects of the disclosure provide embodiments, variations, and examples of methods for generating a spatial map of a distribution of targets of a sample by a set of processes, where the set of processes can include: receiving a sample at a substrate comprising a distribution of functionalized particles, each of the distribution of functionalized particles including a stochastic barcode sequence paired with a position on the substrate (with decoding of the positions of the stochastic barcode sequences prior to use for target mapping); promoting interactions between the distribution of targets of the sample and the distribution of functionalized particles (e.g., upon transmitting heat to a surface of the substrate opposite the distribution of functionalized particles for frozen samples); applying a set of reactions to the sample at the substrate; obtaining a set of sequences of a population of molecules generated from the set of reactions, the set of sequences associated with the distribution of targets labeled using the stochastic barcode sequences of the distribution of functionalized particles, and returning a set of positions of the distribution of targets upon processing the set of sequences.

Aspects of the disclosure also provide embodiments, variations, and examples of systems, methods, and compositions configured for high levels of parallel sample processing, where, upon receiving a sample at a substrate, the sample can be re-frozen (e.g., at 0° C., at −20° C., at −80° C., etc.) for a duration of time prior to performing subsequent processing steps (e.g., post-thaw) for target mapping, without significant degradation in mapping performance (e.g., in relation to performance and quality metrics described).

In relation to quality metrics, aspects of the disclosure also provide embodiments, variations, and examples of systems, methods, and compositions that achieve threshold levels of performance in relation to various quality metrics. In examples, the invention(s) can achieve one or more of: number of paired end sequencing reads greater than a threshold level (e.g., greater than 100,000,000, greater than 200,000,000, greater than 500,000,000, etc.); percentage of read pairs having proper structure greater than a threshold percentage (e.g., 60%, 70%, 80%, 90%, 99%, etc.), where proper structure is determined by comparing the read sequence to the actual synthesized sequence (e.g., order of barcode regions, universal primer regions, unique molecule identifiers, polyT tails, etc.); total number of barcode sequences read per substrate greater than a threshold (e.g., 40,000, 50,000, 60,000, 70,000, 80,000, 90,000, 100,000, etc.); percentage of barcode sequences recovered greater than a threshold (e.g., 60%, 70%, 80%, 90%, 99%, etc.); percent of proper reads matched to a barcode sequence greater than a threshold (e.g., 60%, 70%, 80%, 90%, 99%, etc.); percent of proper reads in genes greater than a threshold (e.g., 60%, 70%, 80%, 90%, 99%, etc.); percent of proper reads matched to barcode sequences and genic sequences greater than a threshold (e.g., 60%, 70%, 80%, 90%, 99%, etc.); percent of raw useful reads (matched to a barcode sequences and genic sequences) greater than a threshold (e.g., 30%, 40%, 50%, 60%, 70%, 80%, 90%, 99%, etc.); average reads per unique molecule identifier (UMI) satisfying a threshold condition; total number of genes in matched bead barcodes greater than a threshold (e.g., 5,000, 10,000, 15,000, 20,000, 25,000, 30,000, 35,000, 40,000, 50,000, etc.); total number of UMIs in matched bead barcodes greater than a threshold (e.g., greater than 5,000,000, 10,000,000, 15,000,000, 20,000,000, 25,000,000, etc.); average reads per bead greater than a threshold (e.g., 100, 200, 300, 400, 500, 600, 700, 800, 900, 1000, etc.); average number of UMIs per bead greater than a threshold (e.g., 100, 200, 300, 400, 500, 600, 700, 800, 900, 1000, etc.); average number of genes per bead greater than a threshold (e.g., 100, 200, 300, 400, 500, 600, 700, 800, 900, 1000, etc.); top percents of reads, UMIs, and/or genes per bead greater than a threshold (e.g., 100, 200, 300, 400, 500, 600, 700, 800, 900, 1000, etc.); average percent of mitochondrial UMIs per bead satisfying a threshold condition; average percent of ribosomal protein UMIs per bead satisfying a threshold condition; average percent of ribosomal RNA UMIs per bead satisfying a threshold condition; and/or other suitable quality metrics.

Aspects of the disclosure also provide embodiments, variations, and examples of systems, methods, and non-naturally-occurring compositions for facilitating capture of target biological material from a sample and characterizing locations of target biological material in space (e.g., two dimensional space, three dimensional space). Such compositions can include materials that have been modified from their natural states (e.g., in terms of providing structural differences from natural compositions). Furthermore, the invention(s) relate to combinations of materials, where the combinations of materials are non-naturally occurring (e.g., there is no naturally occurring counterpart to the compositions described and claimed).

Aspects of the disclosure also provide embodiments, variations, and examples of improved manufacturing methods for generating systems for characterizing locations of target analytes in space, in relation to efficiently generating multiple system units for kitting purposes.

Aspects of the disclosure also provide embodiments, variations, and examples of systems, methods, and compositions for location characterization in multiple dimensions, where particles of the composition(s) described can be implemented in monolayer form (e.g., with manufacturing processes to apply composition units in monolayer or near-monolayer form, with systems that apply magnetic or other forces to form particle monolayers, etc.), with sample (e.g., tissue, cells) positioned adjacent the monolayer for subsequent processing and mapping. Alternatively, particles of the composition(s) described can be infused into a sample/specimen (e.g., by magnetic force, by electroporation, by using vectors, etc.). Alternatively, particles of the composition(s) described can be coupled to a surface of a sample/specimen (e.g., by chemical binding, by magnetic binding, by other binding), in order to enable surface mapping. Alternatively, particles of the composition(s) described can be guided or otherwise retained in 3D structures (e.g., in grids, in non-grid structures), such as microwells, microarrays (e.g., with nucleic acids capturing particles), scaffolds (e.g., hydrogels), or other 3D structures. In a related application, physical or other forces can be used to define structures (e.g., close packed structures) for distributions of particles that interact with samples to enable mapping. Alternatively, in relation to location characterization in multiple dimensions, particles of composition(s) described can be randomly distributed in space.

Aspects of the disclosure also provide embodiments, variations, and examples of systems, methods, and compositions for applications in spatial transcriptomics. Compositions, methods, and systems described can be used for mapping of targets in a sample over time, in order to understand disease pathology and progression (e.g., spread of targets and changes in expression over time).

Aspects of the disclosure also provide embodiments, variations, and examples of systems, methods, and compositions for mapping of nuclei targets of a sample, along with other targets (e.g., cytoplasmic targets, protein targets, etc.) and/or cell types and/or cell subtypes of the sample, using distributions of functionalized particles, with molecules functionalized for capturing different target types. Upon capture, recovery, and amplification of nuclei targets, further processing steps can be performed in order to generate characterizations, including but not limited to methylation status, epigenetic aspects (e.g., control of nuclear architecture, epigenetic changes in relation to previous instances of sampling or sampling from related sources), chromatin accessibility using transposase-accessible chromatin with sequencing ATAC-Seq, cytotoxicity status, and/or other characterizations.

Aspects of the disclosure also provide embodiments, variations, and examples of systems, methods, and compositions for mapping of targets of an FFPE sample according to a set of processes including: deparaffinization of the sample (prior to application to a substrate with functionalized particles, after application to a substrate with functionalized particles), optionally permeabilizing the FFPE sample, capture/tagging of nuclei targets of the FFPE sample along with cytoplasmic targets of the sample (e.g., using cleavable functionalized molecules described in further detail below), retrieving nuclei targets (e.g., with magnetic bodies of functionalized particles implemented, with sample homogenization, with exposure of nuclei using detergents, with electroporation, etc.) thereby retrieving nuclei targets with improved recovery rates for FFPE samples, and performing downstream analyses and mapping of multiple target types from the FFPE sample.

In one variation, a method for mapping nuclei targets of an FFPE sample can include: receiving an FFPE sample at a substrate including a distribution of functionalized particles for tagging nuclei targets of the FFPE sample; drying the FFPE sample and the substrate for a duration of time; performing a deparaffinization operation upon the FFPE sample at the substrate; performing a reverse crosslinking operation with the FPPE sample at the substrate; photocleaving linkers of functionalized molecules of the distribution of functionalized particles, thereby releasing the functionalized molecules for tagging nuclei targets of the FFPE sample; performing a tissue dissociation and nuclei isolation operation upon the FFPE sample; and generating a spatial map of the nuclei targets of the FPPE sample upon sequencing molecules of the FFPE sample after the tissue dissociation and nuclei isolation operation.

In variations, processing an FFPE sample for target mapping (e.g., of cytoplasmic targets, of nuclei targets, etc.) can include dissolving extracellular matrix (ECM) of the FFPE sample (e.g., with collagenase); performing a reverse crosslinking operation in order to reverse crosslinks of molecular components of the FFPE sample (e.g., prior to polyadenylation of nucleic acid targets of the FFPE sample); implementation of high pH buffers for facilitate polyadenylation and/or deparaffinization of FFPE samples; and performing other suitable steps to increase accessibility of RNA material for tagging and subsequent mapping.

In variations, increasing accessibility of nucleic acid targets (e.g., RNA material of an FFPE sample) can include improving efficacy of nucleic acid extraction from a sample, preventing nucleic acid losses into buffers prior to performing hybridization steps, and increasing efficacy of reverse crosslinking steps. In one such variation, increasing accessibility of nucleic acid targets can include performing RNA processing in situ, followed by performing hybridization of targets with molecules of a distribution of functionalized particles (as described herein). In one example, the method can include: increasing accessibility of RNA targets of a sample, generating cDNA copies of the RNA targets upon performing a reverse transcription operation, adding a capture site to 3′ ends of the cDNA copies with template switching oligonucleotides (TSOs), digesting RNA of the sample, permeabilizing cells of the sample, capturing the cDNA copies with the capture sites; and extending molecules of the functionalized particles with the cDNA copies.

Aspects of the disclosure also provide embodiments, variations, and examples of systems, methods, and compositions for multi-omic characterizations of a sample, with mapping of cytoplasmic targets, nuclei targets, protein/antibody targets (with oligo-coupled capture components for protein targets), cluster regularly interspaced short palindromic repeats (CRISPR) targets (e.g., guide RNAs with A-tails or other features that can be captured and amplified), and other targets tagged from the same sample.

Applications of the methods described can be used to deepen characterizations of various tissue types. For instance, in relation to neurological tissue or neurological cells, mapping of multiple target types of the sample can characterize neurons, neuron subtypes, neuron membrane aspects, neuron nuclei aspects, dendritic aspects, axon aspects, oligodendrocyte aspects, hillock aspects, myelin sheath aspects, node of Ranvier aspects, synaptic end bulb aspects, axon terminal aspects, and thus neuron processes performed. In relation to other tissue or cell types, mapping of multiple target types of the sample can characterize cell and tissue subcomponents, as well as cellular functions performed.

Aspects of the disclosure also provide embodiments, variations, and examples of systems that can perform complex characterizations, without requiring traditional platforms for performing such characterizations. For instance, in relation to performing single cell characterizations (which traditionally require complex platforms for single cell partitioning, spatial characterization platforms, and sequencing platforms), the disclosure can streamline operations for performing such characterizations. In one embodiments, samples of cells, nuclei, or other cellular components can be barcoded, and portions can be applied to a substrate with functionalized particles (e.g., after forming a suspension with such components, after centrifugation, after purification, after enrichment, after freezing, after slicing, etc.) for target capture, sequencing, and mapping, without involvement of complex single cell processing setups (e.g., traditionally involving microwells or other partitioning technologies).

As described herein, functionalized particles can be replaced or supplemented with functionalized features, where functionalized features can include spots (e.g., printed spots, spots provided on a substrate, oligonucleotide spots), wells, pits, other recesses, protrusions, columns, beads, nanotubes, or other features on a substrate.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference. Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### BRIEF DESCRIPTION OF THE DRAWINGS

FIG. 1A depicts a schematic of an embodiment of a system for characterizing locations of target analytes in space.

FIG. 1B depicts variations of substrates of a system for characterizing locations of target analytes in space.

FIG. 1C depicts variations of structural features for target capture and spatial mapping, with resulting performance differences.

FIG. 1D depicts variations of particle subpopulations and molecules for target capture, of an embodiment of a system for characterizing locations of target analytes in space.

FIG. 1E depicts variations of molecule types for target interactions, of an embodiment of a system for characterizing locations of target analytes in space.

FIG. 2A depicts a schematic of an embodiment of a support structure for a system for characterizing locations of target analytes in space.

FIG. 2B depicts a schematic of a variation of a support structure for a system for characterizing locations of target analytes in space.

FIG. 3 depicts a schematic of a kit including elements for characterizing locations of target analytes in space.

FIG. 4A depicts a flowchart of an example workflow of a system for characterizing locations of target analytes in space.

FIG. 4B depicts schematics of example workflow steps related to steps shown in FIG. 4A.

FIG. 5 depicts schematics of another variation of a support structure for a system for characterizing locations of target analytes in space and method of use.

FIG. 6 depicts a flowchart of an embodiment of a method of manufacturing a system for characterizing locations of target analytes in space.

FIG. 7A depicts variations of templates used in manufacturing a system for characterizing locations of target analytes in space.

FIG. 7B depicts a variation of a substrate with multiple distributions of functionalized particles.

FIG. 8A depicts an example of separation of units of a system during manufacturing.

FIG. 8B depicts an example of separation of units of a system during manufacturing.

FIG. 9A depicts a flowchart of an embodiment of a method for characterizing locations of target analytes in space.

FIG. 9B depicts a schematic of an embodiment of a method for target characterization.

FIG. 9C depicts a schematic of an embodiment of a method for sample processing, for generation of a spatial map of a tissue sample.

FIG. 10 depicts an example of processing steps associated with characterizing a distribution of single cells/particles/analytes dispersed across a medium or scaffold.

FIG. 11 illustrates exemplary maps of multiple target types from the same sample processed with a unit of the system.

FIG. 12 illustrates an exemplary smearing artifact and background artifact.

FIG. 13A depicts an exemplary system configuration for preventing smearing artifacts and/or background artifacts.

FIGS. 13B and 13C depict variations of methods for preventing smearing artifacts and/or background artifacts.

FIG. 14 illustrates an exemplary system configuration for retaining, isolating, and/or partitioning sample components.

FIG. 15 illustrates a computer system that is programmed or otherwise configured to implement methods provided herein.

### DETAILED DESCRIPTION OF THE INVENTION(S)

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

### 1. General Overview

The present disclosure covers systems, devices, methods performed by such systems and devices, and methods of manufacturing and assembling such devices.

Generally, embodiments of the methods, systems, and compositions provide mechanisms for efficient capture and labeling of target material (e.g., DNA, RNA, miRNA, proteins, small molecules, single analytes, multianalytes, etc.) in order to enable analyses for characterizing locations of target material in space. For nucleic acid targets, capture probes of compositions described can include complementary molecules to the nucleic acid targets. For protein targets or small molecule targets, capture probes of the compositions described can include antibodies or aptamers conjugated with specific nucleic acid sequences for detection.

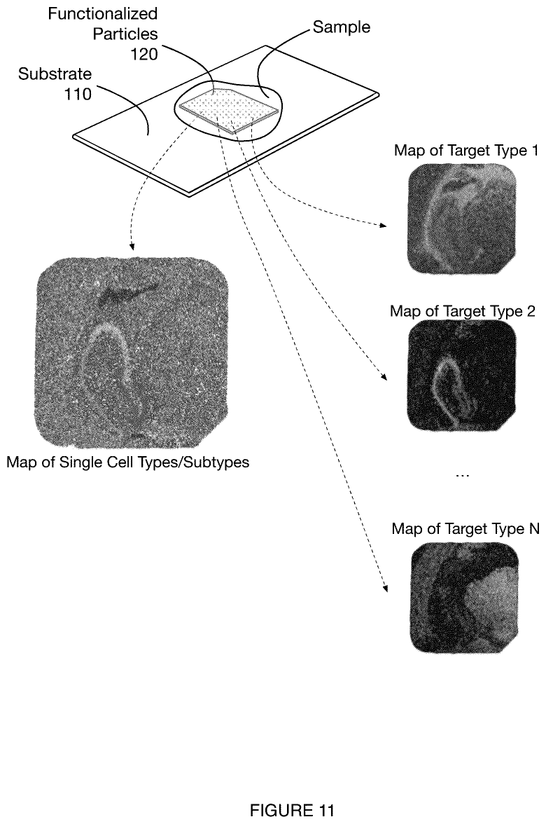

Targets can include cytoplasmic targets, intracellular targets, or other targets on the surface of or within a cell (or components of a cell). For instance, targets can include cytoplasmic targets and targets of or associated with nuclei of cells from the same tissue, such that simultaneous mapping of multiple target types in a highly parallel manner can be achieved (see example in FIG. 11). In one embodiment, spatial labeling of nuclei of a sample can include: processing a sample comprising a set of nuclei with a substrate comprising a distribution of functionalized particles (e.g., with decoding of positions of the functionalized particles by way of stochastic/spatial barcodes, as described below); tagging the set of nuclei with the stochastic/spatial barcodes (e.g., upon cleaving functionalized molecules from the functionalized particles); isolating nuclei of the set of nuclei and/or other targets of the sample; and determining positions of nuclei of the set of nuclei upon sequencing molecules generated from the distribution of functionalized particles. Optionally, in some embodiments, methods can include capture (e.g., microfluidic capture, capture within microwells, capture within partitions, capture within droplets of an emulsion) and barcoding of nuclei targets (e.g., mRNAs, other nuclei targets) and spatial barcodes after isolation of nuclei. Identification of nuclei targets can further optionally be performed using optical detection of nuclei targets tagged with probes during capture and barcoding of nuclei, without sequencing.

In relation to tissue processing, the disclosure provides methods, systems, and devices for spatially labeling targets (e.g., cytoplasmic targets) that are exposed during tissue processing (e.g., tissue slicing, tissue sectioning), as well as spatially labeling of individual nuclei and/or other intracellular components. Methods described can include determining locations of cell bodies (e.g., based upon nuclei positions) of a sample, and performing single-cell analysis techniques in coordination with spatial analysis of single-cell and other targets, where analysis techniques can include single nucleus RNA-seq (snRNA-seq), t-cell receptor (TCR) analyses, b-cell receptor (BCR) analyses (e.g., with receptor-ligand characterizations), ATAC-seq for assessment of chromatin accessibility, processing of nuclei and non-nuclei targets of formalin-fixed and paraffin-embedded (FFPE) samples, analysis of nuclear DNA, analysis of nuclear proteins, and/or other analyses.

The systems, methods, and devices disclosed herein can provide several additional benefits over other systems and methods, and such systems, methods, and devices are further implemented into many practical applications across various disciplines.

The systems, methods, and devices solve cell segmentation issues and sensitivity issues associated with mapping cell-associated targets (e.g., cytoplasmic targets, nuclei-associated targets, etc.). The systems, methods, and devices solve cell segmentation issues and sensitivity issues associated with mapping of targets in human tissue.

The systems, methods, and devices further improve accuracy of spatial mapping of nuclei targets. In embodiments where a nucleus is tagged using cleavable molecules of functionalized particles, tagging of the nucleus can involve tagging the nucleus with different cleavable molecules associated with different spatial positions. As such, determining the position of the nucleus can involve determining the position based upon a subset of positions corresponding to a subset of stochastic barcodes of molecules that tagged the nucleus. The position of the nucleus can be determined from an average position of the subset of stochastic barcodes (e.g., a centroid of positions of the subset of stochastic barcodes). In variations, nuclei can be tagged using a combination of cleavable and non-cleavable molecules, such that positions of the nuclei can be determined from stochastic/spatial barcode positions of cleavable and non-cleavable molecules (e.g., as a weighted centroid of positions, where positions of non-cleavable components are weighted more heavily than positions of cleavable components).

In relation to capture and mapping of nuclei targets, the systems, methods, and compositions described can generate spatial maps with improved recovery rate for nuclei targets (e.g., in relation to actual numbers of nuclei targets present) to greater than 15%, greater than 20%, greater than 25%, greater than 30%, greater than 35%, greater than 40%, greater than 45%, greater than 50%, greater than 60% or greater. In particular, nuclei retention is typically poor due to losses during sample processing, capture of nuclei (e.g., using microfluidic approaches), and isolation of nuclei, and the disclosure provides methods for improved recovery rate and retention of nuclei.

In relation to tagging of nuclei and/or other targets of a sample with functionalized molecules of functionalized particles, functionalized molecules can include unique molecular identifiers (UMIS). The disclosure provides embodiments, variations, and examples of systems, methods, and compositions for recovering greater than 2000 UMIs per nucleus/target, greater than 2500 UMIs per nucleus/target, greater than 3000 UMIs per nucleus/target, greater than 3500 UMIs per nucleus/target, greater than 4000 UMIs per nucleus/target, greater than 4500 UMIs per nucleus/target, greater than 5000 UMIs per nucleus/target, greater than 5500 UMIs per nucleus/target, greater than 6000 UMIs per nucleus/target, greater than 6500 UMIs per nucleus/target, greater than 7000 UMIs per nucleus/target, greater than 7500 UMIs per nucleus/target, greater than 8000 UMIs per nucleus/target, greater than 8500 UMIs per nucleus/target, greater than 9000 UMIs per nucleus/target, greater than 9500 UMIs per nucleus/target, greater than 10,000 UMIs per nucleus/target, greater than 20,00 UMIs per nucleus/target, or greater. As such, systems, methods, and devices described can achieve higher UMI capture per nucleus/target, in relation to existing single-cell techniques and other state-of-the-art techniques.

The systems, methods, and devices can generate spatial maps of a set of targets of a sample, where the spatial maps have unprecedented resolution performance, ability to map multiple sets of targets for different sample and tissue types, and satisfy quality metrics for high resolution mapping.

The systems, methods, and devices also are designed to promote ease of use by end user(s), in relation to processing different tissue types and/or various sample types (e.g., involving natural and synthetic scaffolds).

The systems, methods, and devices provide and implement non-naturally occurring compositions for facilitating capture of target biological material from a sample and characterizing locations of target biological material in space (e.g., two dimensional space, three dimensional space). Such compositions can include materials that have been modified from their natural states (e.g., in terms of providing structural differences from natural compositions). Furthermore, the invention(s) relate to combinations of materials, where the combinations of materials are non-naturally occurring (e.g., there is no naturally occurring counterpart to the compositions described and claimed).

The systems, methods, and devices provide improved manufacturing methods for generating systems for characterizing locations of target analytes in space.

The systems, methods, and devices provide improved characterization of locations of targets in multidimensions (e.g., 2D, 3D, 4D with a time component), in relation to one or more of: whole tissue structures, tissue pieces (e.g., as in histology, in relation to biopsied tissues, in relation to seeded natural scaffolds, in relation to seeded synthetic scaffolds (e.g., cell-seeded hydrogel scaffolds, cell-seeded polaxamer scaffolds, etc.) in relation to frozen tissue specimens, in relation to formalin-fixed and parafin-embedded (FFPE) specimens, etc.), organs, whole organisms, cell suspensions, single cells, organelles, within organelles, in relation to mitochondrial targets, viruses, microorganisms, and other natural structures. Cells can include mammalian cells, bacteria, microbes, plant cells, fungal cells, or other cells/cell-like components. Location characterization can additionally or alternatively be performed in relation to non-naturally occurring structures, such as microwells, microarrays, scaffolds, and other non-naturally occurring structures. For instance, the invention(s) can have in situ and/or in vivo applications, with infusion of functionalized particles into a sample (e.g., into a cell, into a tissue, into an organ, etc.). Examples of infusion can include one or more of: injection, electroporation, use of vectors (e.g., viral vectors), and other infusion methods.

In relation to location characterization in multiple dimensions, particles of the composition(s) described can be implemented in monolayer form (e.g., with manufacturing processes to apply composition units in monolayer or near-monolayer form, with systems that apply magnetic or other forces to form particle monolayers, etc.), with sample (e.g., tissue, cells) positioned adjacent the monolayer for subsequent processing and mapping. Alternatively, particles of the composition(s) described can be infused into a sample/specimen (e.g., by magnetic force, by electroporation, by using vectors, etc.). Alternatively, particles of the composition(s) described can be coupled to a surface of a sample/specimen (e.g., by chemical binding, by magnetic binding, by other binding), in order to enable surface mapping. Alternatively, particles of the composition(s) described can be guided or otherwise retained in 3D structures (e.g., in grids, in non-grid structures), such as microwells, microarrays (e.g., with nucleic acids capturing particles), scaffolds (e.g., hydrogels), or other 3D structures. In a related application, physical or other forces can be used to define structures (e.g., close packed structures) for distributions of particles that interact with samples to enable mapping. Alternatively, in relation to location characterization in multiple dimensions, particles of composition(s) described can be randomly distributed in space.

The systems, methods, and devices provide improved applications in spatial transcriptomics. For instance, compositions, methods, and systems described can be used for mapping of targets in a sample over time, in order to understand disease pathology and progression (e.g., spread of targets and changes in expression over time).

Additionally or alternatively, the systems, devices, or methods described can confer any other suitable benefit.

### 2. Systems

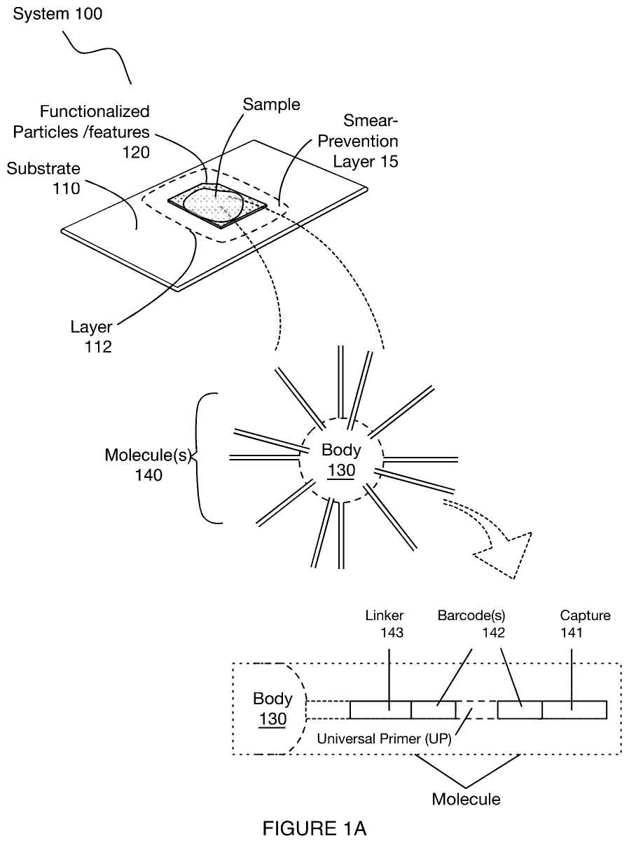

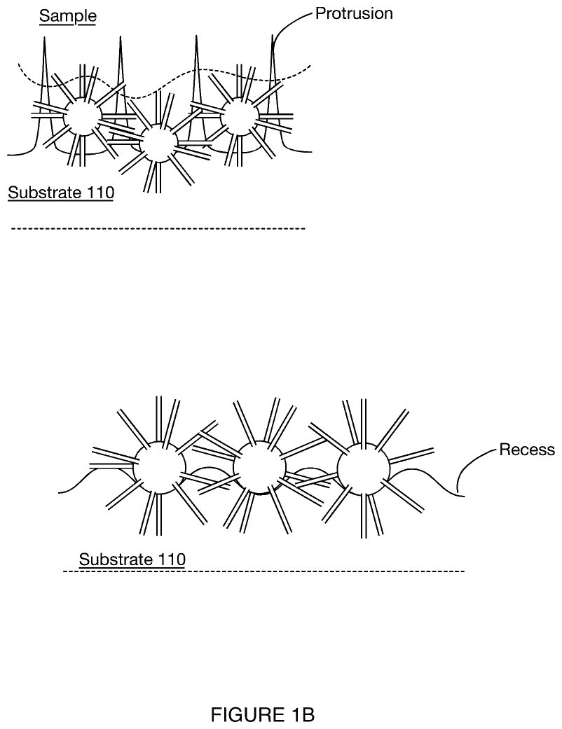

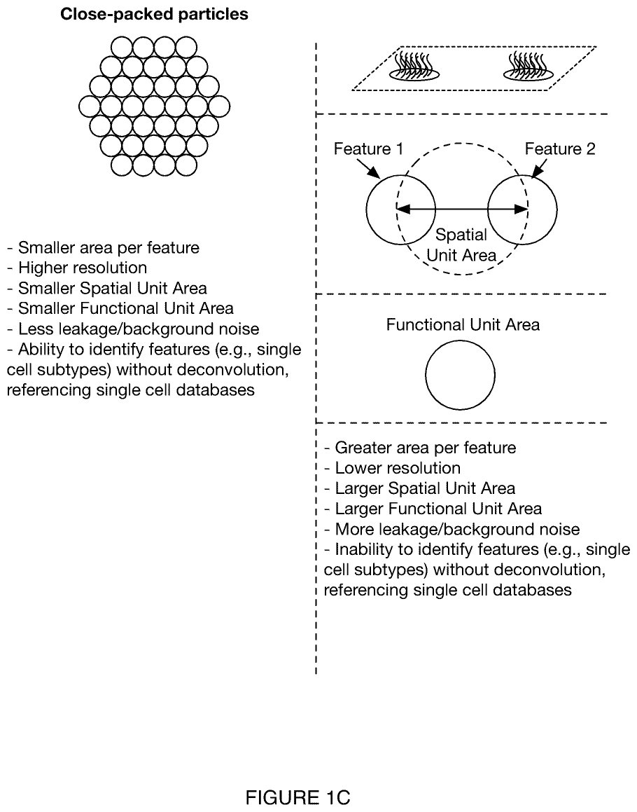

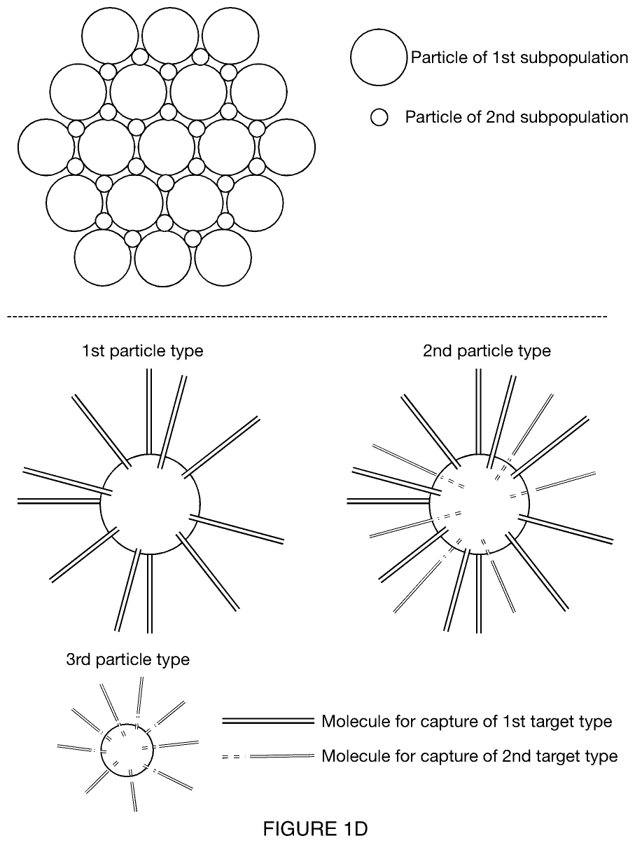

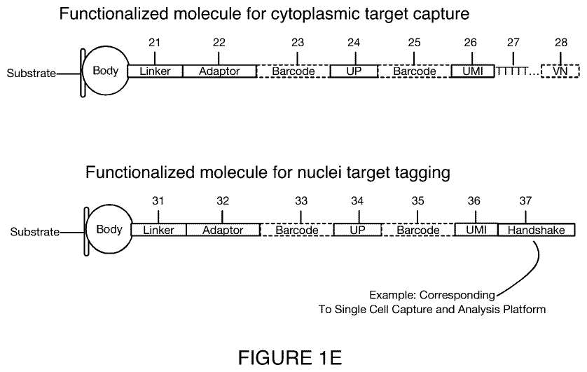

As shown in FIG. 1A, a system 100 for characterizing locations of target analytes of a sample, can include: a substrate 110; and a distribution of functionalized particles 120 coupled to the substrate, wherein each of the distribution of functionalized particles includes: a body 130 reversibly coupled to the substrate, and one or more molecules 140 coupled to the body, the one or more molecules 140 including at least: a capture segment 141, a barcode segment 142, and optionally, a first cleavable linker 143 coupled to the body. The system 100 functions to interact with a sample and/or target analytes of a sample, in order to enable characterization of the location(s) of the target analytes in space.

In embodiments, the target analytes can include one or more of: nucleic acid material (e.g., DNA, RNA, miRNA, etc.), protein material, amino acid material, other small molecules, other single analytes, other multi-analytes, and/or other suitable target material of a sample. In embodiments, the sample can include whole tissue structures, tissue portions (e.g., histological tissue slices, formalin-fixed paraffin-embedded (FFPE) tissue, frozen tissue, biopsied tissues, fresh frozen plasma, seeded natural scaffolds, seeded synthetic scaffolds, etc.), organs, whole organisms, organoids, cell suspensions (e.g., frozen cell suspensions that are separated prior to processing with the system, cell suspensions retained in a medium/hydrogel medium, etc.), nuclei suspension, single cells, organelles, sub-organelle structures, intra-organelle components, mitochondrial targets, viruses, microorganisms, and other samples.

In some non-limiting examples, sample material from which targets can be captured can include one or more of: nervous system biological material, cardiovascular system biological material, integumentary system biological material, skeletal system biological material, muscular system biological material, respiratory system biological material, digestive system biological material, endocrine system biological material, urinary system biological material, and reproductive system biological material. Cellular material can be associated with normal and diseased states, including one or more of: cancer cells, circulating tumor cells, metastatic cells, benign cells, or any combination thereof. In embodiments, the sample can include solid/contiguous tissue material obtained from a subject.

Details of the system 100 and use thereof are described in further detail in the following sections.

### 2.1 System-Substrate

The substrate 110 functions to provide one or more surfaces onto which the distribution of functionalized particles 120 is patterned or otherwise deposited (as described below). The substrate 110 thus functions to support the distribution of functionalized particles 120 in a reliable manner during sample handling and processing. The substrate 110 can additionally or alternatively function to support mechanisms for controlled interactions with the distribution of functionalized particles (e.g., with respect to controllable binding and release mechanisms, etc.). The substrate 110 can additionally or alternatively function to facilitate detection of optical signals generated from interactions between the distribution of functionalized particles 120 and captured target analytes of the sample, by having suitable optical characteristics for transmission of light signals to an optical signal sensing apparatus. However, in variations, the substrate 110 can be processed to release functionalized particles, which are processed to characterize the distribution of functionalized particles away from the substrate 110. The substrate 110 can additionally or alternatively function to enable transmission of heat to a sample interacting with the system 100 during use, in order to promote interactions between the target analytes and the distribution of functionalized particles 120 at the substrate 110. Additionally or alternatively, the substrate 110 can have other suitable functionality.

In one embodiment, the substrate 110 is composed of glass/silica (e.g., a borosilicate glass), which offers desired properties for manufacturing (e.g., in relation to surface functionalization, in relation to processing, in relation to separation of composition units, etc.), thermal characteristics (e.g., in terms of thermal conductivity, electrical characteristics (e.g., in terms of supporting charge, in terms of electrical conductivity, etc.), optical characteristics (e.g., providing mechanisms for optical recognition, characterized by one or more optical features encoding a set of nucleic acid bases, the set of nucleic acid bases identifiable upon detection of the one or more optical features, etc.), magnetic properties (e.g., in relation to providing or supporting magnetic fields for manipulation of sample components and/or aspects of the distribution of functionalized particles 120), biocompatibility characteristics, and/or other suitable characteristics. Alternatively, the substrate 110 can include, or be composed of one or more of: plastic/polymer materials (e.g., acrylic, cyclic olefin polymer, polycarbonate, poly(methyl methacrylate) (PMMA), cyclo olefin polymer (COP), polystyrene, polypropylene, polyethylene terephthalate glycol-modified (PEGT), etc.); ternary compositions (e.g., indium tin oxide); and/or other suitable materials.

In variations, the substrate 110 has a characteristic roughness less than or equal to 1 micrometer (e.g., 0.8 micrometer), but can alternatively have another suitable roughness. For instance, variations of the substrate 110 can have a desired roughness (e.g., greater than 1 micrometer) to provide a desired texture or serve other suitable functionality.

Additionally or alternatively, the substrate 110 can be flexible (e.g., composed of a flexible material) in order to enable applications involving flexible application to a sample surface (e.g., wrapping around a tissue body, etc.).

In some variations, as shown in FIG. 1B, the substrate 110 (and/or functionalized particle surfaces) can include a set of protrusions establishing an interface between the sample and the distribution of functionalized particles during use. As such, during operation, protrusions of the substrate 110 can be configured to extend into the sample (e.g., into tissue) to promote desired interactions between deeper portions of the sample and the distribution of functionalized particles at the substrate 110. In a related variation, the protrusions can be hollow (e.g., as in microneedles), to aid transmission of fluid material to the sample (e.g., for sample processing) and/or to promote target analyte capture.

Additionally or alternatively, as shown in FIG. 1B, the substrate 110 can include a set of recesses configured to receive or otherwise support the distribution of functionalized particles 120, for instance, for positioning of the distribution of functionalized particles 120 at the substrate 110 in a desired configuration (e.g., patterned manner, with desired density, in a monodisperse manner, in monolayer, etc.).

Additionally or alternatively, the substrate 110 can include or be positioned adjacent to a set of fiducials, where the set of fiducials can provide observable markings for manufacturing (e.g., in relation to scribing/sawing of the substrate to separate units). Additionally or alternatively, the set of fiducials can define addressable locations of the system 100/distribution of functionalized particles for characterizing locations of target analytes of the sample captured using the system 100.

Additionally or alternatively, the substrate 110 can be optically recognizable (e.g., such that the substrate can be observed with optical apparatus to provide a signal). In variations, the substrate can be characterized by one or more optical features encoding a set of nucleic acid bases, the set of nucleic acid bases identifiable upon detection of the one or more optical features.

### 2.2 System-Functionalized Particles

As shown in FIG. 1A, the system 100 further includes a distribution of functionalized particles 120 coupled to the substrate, in order to enable analyses for characterizing locations of target material in space. The distribution of functionalized particles 120 functions to capture target analytes from the sample, and to provide functionality for decoding aspects of the target analytes and/or locations of each target analyte unit in space, upon sequencing. each functionalized particles 120 includes a body 130 (e.g., a body reversibly coupled to the substrate 110), and one or more molecules 140 coupled to the body, the one or more molecules 140 including at least: a capture segment 141, a barcode segment 142, and a first cleavable linker 143 coupled to the body.

In embodiments, the distribution of functionalized particles 120 is arranged at the substrate 110 (e.g., using a templating process, using another suitable process) with a circular footprint; however, in other variations the distribution of functionalized particles 120 can be arranged with other suitable morphology (e.g., an ellipsoidal footprint, a rectangular footprint, a polygonal footprint, an amorphous footprint, etc.). In one such example, the distribution of functionalized particles 120 is arranged with a square footprint having a corner notch for orientation purposes. In still other variations, the distribution of functionalized particles 120 can be patterned onto the substrate 110 in an arrangement corresponding to the sample(s) being processed using the system 100. For instance, in some variations, the distribution of functionalized particles 120 can be patterned in a manner corresponding to a characteristic sample shape (e.g., tissue biopsy shape), characteristic sample structural features (e.g., tissue fiber orientations), characteristic sample container shape (e.g., tube shape, well shape, etc.), and/or other suitable feature.

In variations, a characteristic dimension (e.g., diameter, width, length, etc.) of the arrangement in bulk of the distribution of functionalized particles 120 can range from 1 to 10 mm (or alternatively, greater than 10 mm), and in specific examples, the characteristic dimension of the distribution of functionalized particles 120 can range from 2 to 4 mm (e.g., in diameter for a circular footprint). The distribution of functionalized particles can have a length or width of 1 millimeter, a length or width of 2 millimeters, a length or width of 3 millimeters, a length or width of 4 millimeters, a length or width of 5 millimeters, a length or width of 6 millimeters, a length or width of 7 millimeters, a length or width of 8 millimeters, a length or width of 9 millimeters, a length or width of 10 millimeters, a length or width of 11 millimeters, a length or width of 12 millimeters, a length or width of 13 millimeters, a length or width of 14 millimeters, a length or width of 15 millimeters, a length or width of 16 millimeters, a length or width of 17 millimeters, a length or width of 18 millimeters, a length or width of 19 millimeters, a length or width of 20 millimeters, an intermediate length or width, or another suitable length or width (e.g., greater than 20 millimeters).

In one example, a distribution of functionalized particles having a footprint of 3 mm×3 mm can be used for simultaneous capture/tagging of nuclei targets and other targets (e.g., cytoplasmic targets). In another example, a distribution of functionalized particles having a footprint of 10 mm×10 mm can be used for simultaneous capture/tagging of nuclei targets and other targets (e.g., cytoplasmic targets).

A substrate can have multiple distributions of functionalized particles arranged as separate arrays.

In examples, larger tiles can be used to accommodate larger tissue samples, in order to generate spatial maps of larger tissue samples. However, the distribution of functionalized particles 120 can alternatively have another suitable characteristic dimension.

Furthermore, a substrate can have more than one distribution of functionalized particles 120, as shown in FIG. 7B, where the distributions (e.g., 120a through 120i shown in FIG. 7B) can be arranged in an array or otherwise arranged. In variations, the distributions can be arranged in a 2×2 array, a 3×3 array, or any other suitable array (ordered or non-ordered) on a substrate. Arrays of distributions of particles can be used to accommodate larger tissue samples, in order to generate spatial maps of larger tissue samples. As such, methods using arrayed distributions can include applying a sample (e.g., tissue sample), to an array of distributions of functionalized particles, and processing the sample according to embodiments, variations, and examples of method steps described.

Furthermore, one or more distributions of functionalized particles at a substrate can include differences in barcode sequences (e.g., each unit of the array can have a different barcode sequence), which can be used for selective amplification and sequencing of different regions of a sample applied to the array. In one example, a region of interest associated with a first unit of the array can be selectively amplified and sequenced using the unique barcode sequence of the unit. If results from the region of interest are promising or otherwise satisfy a condition (e.g., quality condition) that warrants additional investigation, then the method can include selective amplification and sequencing of other regions of the sample, using the unique barcode sequences of the other units.

In more detail, implementation of multiple distributions of functionalized particles (e.g., different sub-arrays each having the same or different footprints) can enable regional subsampling (e.g. selective amplification, interrogation, and/or detection of targets at a specific sub-array). Different sub-arrays can be configured to capture different target types (e.g., a first sub-array can be configured to capture a first target type, and a second sub-array can be configured to capture a second target type). Alternatively, different sub-arrays can each be configured to capture multiple target types (e.g., a first sub-array can be configured to capture multiple target types). In one example, in order to reduce overall experiment cost, a first sub-array with captured targets can be interrogated in relation to a first target type (e.g., cytoplasmic targets, nuclei targets, or other targets), and based upon such interrogation, the same or other sub-arrays can be interrogated for characterizations of the same or other target types (e.g., cytoplasmic targets, nuclei targets, or other targets). Such a configuration thus enables initial analyses to be performed, which can guide decisions as to whether additional analyses should be performed.

Selective amplification can be enabled by implementation of functional molecules for capturing different target types, where molecules can have different PCR handles corresponding to different target types intended to be captured (as described in relation to functional molecules described in further detail below).

The distribution of functionalized particles 120 is preferably arranged at the substrate 110 with a high degree of density (e.g., hexagonal close packing, rectangular close packing, near-close packing, etc.). In specific examples, the distribution of functionalized particles is characterized by a high level of occupancy at the surface of the substrate 110. In relation to close packing of particles (e.g., random close packing), the packing density at the substrate can be from 55% to 74%, such that the empty or dead space between particles is from 26% to 45%. As such, the configuration of the distribution of functionalized particles achieves minimal dead space, as permitted by physics. Additionally or alternatively, in relation to implementation of functionalized particles having different subpopulations of body sizes, interstitial spaces or other gaps between particles of a first size can be covered with particles of a second size, in order to further increase density of packing and thus, resolution (e.g., as in FIG. 1D).

However, the distribution of functionalized particles can be characterized by another suitable percent occupancy. The distribution is preferably also monodisperse (e.g., uniformly distributed and with particles of substantially uniform size, with a critical distance between particles below a threshold); however, the distribution can alternatively be non-monodisperse/random. As such, application of the distribution of functionalized particles can be random or guided by morphology of the substrate (e.g., with meshes, with wells, with protrusions, with recesses, with textures, etc.).

Furthermore, the distribution of functionalized particles is preferably arranged at the substrate 110 in a monolayer (e.g., without stacking); however, in variations, the distribution of functionalized particles 120 can be arranged at the substrate 110 with a different degree of density (e.g., non-packed) and/or in non-monolayer format. In one such variation, the distribution of functionalized particles can be arranged in one or more sub-arrays (e.g., patterned for a specific application of use). In examples, the sub-arrays can include a different sub-arrays functionalized for different target analytes, different forms of target analytes (e.g., such as for different epitopes), control regions, or other suitable regions. Additionally or alternatively, multiple distributions can be arranged at a single substrate (e.g., an array or matrix of distributions of functionalized particles, arranged in discrete zones at the substrate).

The distribution of functionalized particles 120 can be coupled to the substrate 110 using a layer 112 to which particles can adhere (e.g., in a reversible manner, in a permanent manner). In variations, the distribution of functionalized particles 120 can be coupled to the substrate 110 by way of the layer 112 (shown in FIG. 1A) in a coupled operation mode, and separated from the substrate 110 in a separated operation mode. The adhesion or pull-off force for separation can range from 200 to 500 nano-Newtons; however, in other variations, the adhesion or pull-off force can be less than 200 nano-Newtons or greater than 500 nano-Newtons.

In variations, separation can be achieved using a detergent that separates the distribution of functionalized particles 120 from the layer 112 and/or substrate 110 directly. As such, by coupling of the distribution of functionalized particles can be provided by at least one of a hydrophobic interaction and a hydrophilic interaction that is reversible by addition of a detergent separation. Additionally or alternatively, separation can be achieved with linkers (e.g., cleavable linkers) coupling the functionalized particles to the layer or substrate (e.g., by a chemical modification at the substrate), where the linkers are configured to be cleaved in response to one or more of: a thermal cleavage mechanism, a pH shift, a photocleaving mechanism, a chemical cleaving mechanism, an enzymatic cleaving mechanism (e.g., as in molecular scissors), separation based upon changes in charge (e.g., as in an electrostatic interaction), or another suitable cleaving/separation mechanism.

Functionalized particles can have an adherence of a threshold force level (e.g., 100 nano-Newtons, 200 nano-Newtons, greater than 200 nano-Newtons, etc.) prior to cleavage.

The layer 112 can be composed of a rubber (e.g., thermoplastic rubber) and/or other suitable polymer, and in specific examples, includes one or more of: an isoprene-based material, a styrene-based material, a propylene-based material, an ethylene-based material, a nylon-based material, and other suitable rubber/polymer materials. However, in other variations, the layer 112 can alternatively be composed of another suitable material.

The layer 112 can be hydrophobic, or can alternatively have a suitable degree of hydrophilicity, in relation to fabrication processes (e.g., using a spraying process, using a vapor deposition process, using a spin-coating process, using a printing process, etc.) and coupling of the distribution of functionalized particles 120 to the layer. The layer 112 can further be composed of a thermoplastic material, or can alternatively be composed of a thermosetting material. The layer 112 can be processed in liquid form (e.g., with suitable solvents) and applied to the substrate 110 using one or more processes (described in further detail below); however, the layer 112 can additionally or alternatively be applied to the substrate 110 in another suitable manner (e.g., as a pre-generated film, using a printing process, using a patterning process, etc.). For instance, in some examples, the layer 112 can be applied to the substrate 110 with a pattern or texture that promotes preferential coupling of the distribution of functionalized particles 120 to the layer 112 with a desired pattern and/or density (e.g., by using hydrophobic characteristics, hydrophilic characteristics, chemical bond characteristics, etc.).

Additionally or alternatively, adherence can be supported and/or reversed using magnetic forces. For instance, as described above, functionalized particles can have or be composed of magnetic materials, and manipulated (e.g., retained in position or separated from a substrate) by application, reversal, and/or removal of magnetic forces.

In relation to manufacturing processes described in further detail below, multiple distributions of functionalized particles can be arranged at a bulk substrate, and separated from each other to create units of the system 100. Additional details of manufacturing are described in more detail below.

Furthermore, during use, and in an application of use involving spatial characterization of target analytes in 3D, stacks of substrates with distributions of functionalized particles can be implemented (e.g., with layering of samples/slices of tissue and units of the system 100). As such, the system 100 can include additional substrates with distributions of functionalized particles (e.g., a second substrate with a second distribution of functionalized particles, a third substrate with a third distribution of functionalized particles, etc.), with layering of sample and reconstruction of 3D volumes by stitching data derived from implementation of the various substrates.

Embodiments, variations, and examples of the distribution of functionalized particles can additionally or alternatively include particle compositions described in U.S. application Ser. No. 17/376,396 filed on 15 Jul. 2021, which is herein incorporated in its entirety by this reference. Such functionalized particles for determining nearest neighbor interactions can thus be provided with substrates or other natural/synthetic structures in order to characterize locations of target analytes in space.

### 2.2.1 Functionalized Particles

Each body 130 of the distribution of functionalized particles 120 functions to provide a surface to which the one or more molecules 140 can be coupled, in order to provide functionalization for target analyte capture, sample processing, and subsequent location characterization operations.

In relation to morphology, the body 130 can have the form of a microsphere. Alternatively, the body 130 can have the form of a non-spherical (e.g., ellipsoidal, prismatic, polyhedral, amorphous, nanotube, etc.) body, where a cross section taken through the body 110 is non-circular. However, the body 130 can alternatively have another suitable form. For instance, in variations, the bodies 130 can be quantum dots responsive to excitation by different types of energy (e.g., wavelength ranges of electromagnetic energy for various applications.

In relation to dimensions, the body 130 can have a diameter (or characteristic width) on the order of nanometers to micrometers in dimension, where particle size determines the resolution of target analyte location characterization. Dimensional characteristics of the body 130 correspond to scales appropriate for characterization of target analyte locations for various structures (e.g., cells, tissues, and organs, sub-cellular structures, whole organisms, other components, etc.). In variations, the body 130 can have a diameter that is sub-micron up through 10 micrometers; however, alternative variations of the body 130 can have other suitable dimensions (e.g., less than sub-micron, greater than 10 micrometers in diameter). In specific examples, the dimensions of the bodies can be from 3-5 micrometers. In relation to body dimensions below 6 micrometers, tissue processing steps (e.g., permeabilization steps) can be optimized to prevent leakage of targets toward particles not proximal to their respective originating positions at the sample. Alternatively, the sample may not be permeabilized, such that the method involves positioning a non-permeabilized tissue sample at the substrate, thereby avoiding target diffusion away from origination positions in the sample, and preventing background noise (i.e., producing high SNR values) during spatial mapping. As such, use of particles having smaller dimensions can still be used to achieve accurate mapping without producing background noise attributed to target leakage or other noise sources.

In variations, the body 130 can have other suitable dimensions. For instance, the body 130 can have a diameter of 1 micrometer, 2 micrometers, 3 micrometers, 4 micrometers, 5 micrometers, 6 micrometers, 7 micrometers, 8 micrometers, 9 micrometers, 10 micrometers, 11 micrometers, 12 micrometers, 13 micrometers, 14 micrometers, 15 micrometers, 16 micrometers, 17 micrometers, 18 micrometers, 19 micrometers, 20 micrometers, intermediate diameter dimensions, or greater diameter dimensions.

In variations, the distribution of bodies can include different subpopulations of bodies/subpopulations of functionalized particles (e.g., as in FIG. 1D). For instance, the distribution of bodies can include a first subpopulation of bodies having a first diameter (e.g., a diameter below 5 micrometers, another suitable diameter) and/or other first characteristic, and a second population of bodies having a second diameter (e.g., a diameter above 5 micrometers, another suitable diameter) and/or other second characteristic. Additionally or alternatively, the distribution of bodies can include a third subpopulation of bodies having a third diameter and/or other third characteristic, a fourth subpopulation of bodies having a fourth diameter and/or other fourth characteristic, and/or other suitable subpopulations of bodies.

In one application, bodies of a first subpopulation can be functionalized (e.g., with a first capture molecule type) for capturing a first target type (e.g., cytoplasmic targets, sample surface targets, mRNAs by polyA/polyT interactions, etc.). Bodies of the first subpopulation can have a first diameter (e.g., a diameter above 5 micrometers, another suitable diameter). Bodies of a second subpopulation can be functionalized (e.g., with a first capture molecule type) for capturing a second target type (e.g., targets comprising nuclei or nuclei-associated targets, for instance, with randomer segments, segments that are gene-specific to nuclei targets, segments that capture extended nuclei targets, segments that capture polyadenylated nuclei targets, or other capture segments for nuclei targets). Bodies of the second subpopulation can have a second diameter (e.g., a diameter below 5 micrometers, another suitable diameter). Bodies of the second subpopulation can be dispersed interstitially (e.g., in spaces between) or amongst the first population at a substrate. As such, in this application, multiple subpopulations of functionalized particles can be implemented in order to capture different targets simultaneously, for characterizations of distributions of different targets using the same system.

In more detail, molecules for capturing a first target type (e.g., cytoplasmic targets, sample surface targets, mRNAs by polyA/polyT interactions, etc.) can include: a linker sequence 21, an adaptor sequence 22 (e.g., for a next generation sequencing platform, for library preparation), a first barcode sequence 23, a UP sequence 24, a second barcode sequence 25, a UMI sequence 26, and a polyT sequence 27 (e.g., dT, dTVN, etc.) or alternative sequence (e.g., sequence comprising Ts and other bases) for tagging of mRNA targets. Molecules structured for mRNA capture can further include a VN anchor 28 including a dV and a dN (i.e., a V sequence including an A, C, or G nucleotide positioned next to an N sequence including an A, G, C, or T nucleotide) at or near 3′ end. The addition of a VN anchor 28 can promote capture of mRNAs further into a polyA portion of the mRNA molecule, closer to 5′ end. The addition of the VN anchor 28 can also support approaches for capturing polyadenylated (A-tailed) nucleic acids (e.g., polyadenylated micro RNAs, polyadenylated small nuclear RNAs, polyadenylated viral RNAs, polyadenylated microbial RNAs, polyadenylated RNAs non-host RNAs, polyadenylated coding and non-coding RNAs, etc.), where polyadenylation can involve use of yeast polyA polymerase or other suitable components for polyadenylation.

An example of a molecule for mRNA capture is shown in FIG. 1E, with sequences positioned in a 5′ to 3′ direction.

In more detail, molecules for barcoding, identification, and capturing a second target type (e.g., targets comprising nuclei or nuclei-associated targets, for instance, with randomer segments, segments that are gene-specific to nuclei targets, segments that capture extended nuclei targets, segments that capture polyadenylated nuclei targets, or other capture segments for nuclei targets) for spatially resolving the second target type can include: a linker sequence 31, a an adaptor sequence 32 (e.g., for a next generation sequencing platform, for library preparation), a first barcode sequence 33, a UP sequence 34, a second barcode sequence 35, a UMI sequence 36, and a handshake sequence 37 for nuclei targets/other targets.

An example is shown in FIG. 1E, with sequences positioned in a 5′ to 3′ direction. Molecules for capturing a second target type can be cleaved and hybridize with capture sequences of particles for single cell analyses, where such particles can be functionalized for co-capture of nuclear mRNA targets as well.

Molecules can have one or more G bases, one or more C bases, one or more T bases, one or more A bases, one or more U bases, one or more R bases, one or more Y bases, one or more K bases, one or more M bases, one or more S bases, one or more W bases, one or more B bases, one or more D bases, one or more H bases, one or more I bases, one or more V bases, one or more N bases, where bases are described with nucleotide symbols. Bases can be arranged in a pattern and/or randomly within a sequence used for interactions with targets of a sample (e.g., with full complementarity to the target, without full complementarity to the target).

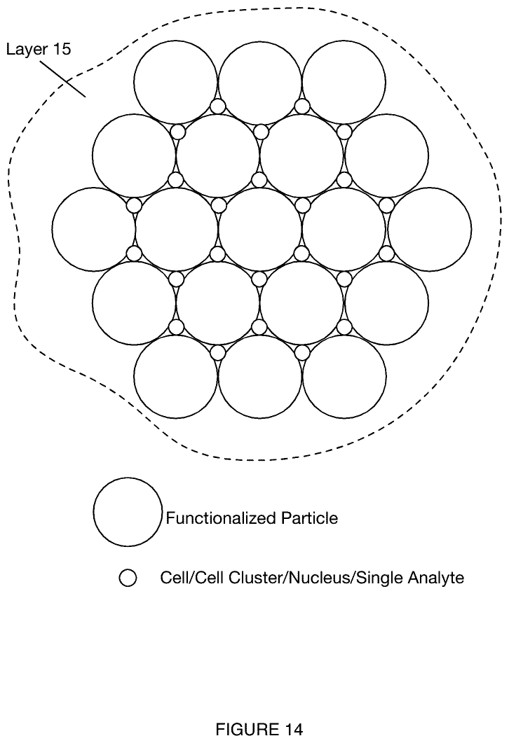

In still other variations, one of which is shown in FIG. 14, a distribution of functionalized particles can be configured, such that spaces between functionalized particles serve as features for retaining, partitioning, and/or isolating sample components (e.g., cells, nuclei, cell clusters, single analytes, grouped analytes, etc.) in position at the substrate, for spatial mapping and other characterizations of targets associated with the isolated sample components. As shown in FIG. 14, the sample components can be individually isolated interstitially in spaces between functionalized particles. Alternatively, sample components can be seated upon regions defined by borders of adjacent functionalized particles. In an example, sample components can include single cells. In another example, sample components can include nuclei (e.g., nuclei tagged with barcodes and isolated, as described).

In variations, sample components that are isolated relative to a distribution of functionalized particles at the substrate can be covered (e.g., sealed), with a layer, such as smear-prevention layer 15 described below. In variations, the smear-prevention layer 15 can be composed of optimal cutting temperature (OCT) compound (e.g., OCT compound alone, OCT compound combined with other process reagents, etc.), an oil, an aqueous material, a mesh, a hydrogel, or another suitable material. Covering can thus serve protective functions with respect to maintenance of cell or other target viability and general sample handling, and/or sample processing functions.

With respect to functionalized particles described, the body 130 can have suitable density characteristics (e.g., with density less than, greater than, or equal to various process liquids associated with processing and characterization operations); porosity (e.g., with pore sizes of 100-2000 Angstroms, etc.); thermal properties (e.g., with respect to melting temperatures, with respect to conductivity, with respect to temperature sensitivity/responsiveness, etc.); physical properties (e.g., with respect to swelling characteristics, with respect to leaching characteristics, with respect to hydrophilicity, with respect to crosslinking, etc.); surface properties (e.g., binding sites for linker molecules, functional chemical groups, charge, etc.); magnetic properties (e.g., magnetic properties, paramagnetic properties, for instance by incorporation of magnetic nanoparticles, etc.); fluorescence properties (e.g., non-fluorescence so as to not interfere with optical-based detection assays, fluorescent/optical feature properties, such as fluorescence-embedded labels, encoding nucleic acid bases identifiable upon detection of the optical features, etc.); buoyant properties (e.g., in order to arrange particles at a surface due to buoyancy, and applying the sample to the buoyant distribution at the surface to which the buoyant particles migrate); mechanical properties (e.g., hardness, rigidity, elastic behavior, viscoelastic behavior, fatigue resistance, fracture resistance, shear strength, compressive strength, tensile strength, rheological behavior, etc.); solubility (e.g., dissolvable in a solvent, etc.); pH sensitivity; and/or other suitable properties, embodiments, variations, and examples of which are described in U.S. application Ser. No. 17/376,396 filed on 15 Jul. 2021, which is herein incorporated in its entirety by this reference.

In relation to composition, the body 130 can be composed of one or more of: polymers (e.g. polystyrene, polystyrene-divinylbenzene, polymethylmethacrylate (PMMA), etc.), hydrogels, silica, silicon, non-porous glass, porous glass, coated glass, agarose, acrylamide, polyacrylamide, iron, steel, or ceramic materials and/or a combination of one or more suitable materials. Different regions of the body 130 can be composed of different materials (e.g., a core region can be composed of a first material and a shell region can be composed of a second material). Additionally or alternatively, the body can be treated or otherwise compatible with phosphoramidite chemistry for synthesis of oligonucleotides onto the body 130. In specific examples, synthesis can be performed by: synthesizing constant sequences in a single column, followed by deprotection, then distribution of bodies across four columns, each configured to add one of an A, T, G, or C phosphoramidite to the bodies. Then, the bodies can be pooled, mixed well, and redistributed across the four columns for addition of an A, T, G, or C phosphoramidite to the bodies. As such, the barcode sequence added to each bead is randomized, but all oligonucleotides on each bead will have the same sequence. Synthesis can implement use of exonucleases to remove or otherwise avoid undesired levels of truncated oligonucleotides of the functionalized particles.

In some embodiments the body 130 can include multiple regions either as multiple shell regions, or in other configurations such as amorphous or ordered spatial arrangements. In still further examples, the body 130 can include or take the form of a polymeric/molecular body (e.g., DNA nanoball, dendrimer, etc.), where, in applications, the dendrimers can be reduced in size to a “functional monomer” (i.e., as a smallest functional molecular assembly unit).

Each body has one or more molecules 140 coupled thereto and structured to provide functionality as described below. The occupancy of molecules at the surface of each particle can be configured to prevent crowding, prevent self-hybridization, and/or enable access of target analytes and/or enzymes as required during processing. In embodiments, the one or more molecules 140 include at least: a capture segment 141, a barcode segment 142, and optionally a first cleavable linker 143 coupled to the body 130 (however, non-cleavable linkers can also be used for functionalization and providing attachment points for oligonucleotides). As described below, the one or more molecules 140 can include or omit regions based upon application of use. The density of the one or more molecules can be at least 10 times more than the amount of target analyte intended for capture from the sample(s), or otherwise configured with another suitable density in other applications of use.

The capture segment 141 functions to capture the target analyte of interest from the sample. The capture segment 141 is preferably positioned at a terminal end of a respective molecule in order to promote interactions with target analytes of the sample; however, the capture segment 141 can alternatively be otherwise positioned along a molecule. In variations, analytes captured by the capture probe 141 can include: nucleic acids (e.g., DNA, mRNA, miRNA etc.), oligonucleotides with polyT for mRNA capture, oligonucleotides without polyT, with a oligonucleotide with polyT attached to other types of molecules (e.g., antibodies, proteins, peptides, chemicals, etc.), antibodies, aptamers, and/or other suitable capture segments.

In one variation, the capture segment 141 can be configured for target mRNA binding (e.g., dT, dTVN for capturing polyA mRNAs). In downstream applications, captured target mRNAs can be processed (e.g., using reverse transcription to append the capture segments 141 with captured mRNAs with cDNA, followed by amplification of synthesized cDNA, followed by sequencing for target readout, etc.). In alternative variations, the capture segment 141 can include functionality for capture of one or more of: DNAs, other RNAs (e.g., miRNA), proteins (e.g., antibodies, using TotalSeq™ molecules), small molecules, single analytes, multianalytes, etc.), and/or other target material (e.g., to generate combinatorial libraries).

The capture segment 141 can additionally or alternatively be a gene-specific sequence to capture a set of specific genes or targets. The analytes can be modified to have a polyA tail (e.g. oligonucleotide-conjugated antibody, aptamer, peptide, etc.), or analyte-specific sequence, such that they can be captured by the capture probe via DNA complementarity. The capture segment 141 can also be a sequence with complementarity to a sequence on other sets of beads.

A first variation of a capture segment 141 can include a polyT sequence 27 (e.g., dT, dTVN, etc.) for capture of mRNA targets (an example of which is shown in FIG. 1E). Molecules structured for mRNA capture can further include a VN anchor 28 (shown in FIG. 1E) including a dV and a dN (i.e., a V sequence including an A, C, or G nucleotide positioned next to an N sequence including an A, G, C, or T nucleotide) at or near 3′ end. The addition of a VN anchor 28 can promote capture of mRNAs further into a polyA portion of the mRNA molecule, closer to 5′ end. The addition of the VN anchor 28 can also support approaches for capturing polyadenylated (A-tailed) nucleic acids (e.g., polyadenylated micro RNAs, polyadenylated small nuclear RNAs, polyadenylated viral RNAs, polyadenylated microbial RNAs, polyadenylated RNAs non-host RNAs, polyadenylated coding and non-coding RNAs, etc.), where polyadenylation can involve use of yeast poly A polymerase or other suitable components for polyadenylation.

A second variation of a capture segment 141 can include a sequence for capturing a second target type (e.g., targets comprising nuclei or nuclei-associated targets, for instance, with randomer segments, segments that are gene-specific to nuclei targets, segments that capture extended nuclei targets, segments that capture polyadenylated nuclei targets, or other capture segments for nuclei targets). An example of a capture sequence 141 for tagging nuclei targets of a sample can include a sequence of 5′ GCTTTAAGGCCGGTCCTAGCAA 3′.

A third variation of a capture segment 141 can include a capture segment configured to support capture of targets in a manner that does not compete with mRNA capture (e.g., by other molecules structured for mRNA capture where the other molecules are coupled to the same particle). A specific example of the third variation of the capture segment 141 can include a sequence of 5′ AAGCAGTGGTATCAACGCAGAGTG 3′. In exemplary use cases, the third variation of the capture segment 141 can support applications involving CRISPR screening, antibody capture, VDJ immune repertoire sequencing, and other applications.

A fourth variation of a capture segment 141 can include a capture segment configured to support 5′ capture of single targets. A specific example of the fourth variation of the capture segment 141 can include a sequence of 5′ TTTCTTATATrGrGrG 3′. In exemplary use cases, the fourth variation of the capture segment 141 can be used to capture 5′ ends of targets by taking advantage of non-templated rC nucleotides.

The capture segment 141 can, however, be configured for capture of other targets.

Related to the variations described, a functionalized particle can include a first subset of molecules with capture segments of a first type and a second subset of molecules with capture segments of a second type. Fabrication of a particle with different subsets of molecule types can include incorporating a mixture of a recognizable element (e.g., phosphoramidite, such as dt-DMT) and a protecting group (e.g., dA-Lev, where the ratio of the recognizable element and the protecting group in the mixture can be used to control the ratio of the first subset of molecules and the second subset of molecules at a particular functionalized particles. The recognizable element and the protecting group can both be configured to couple to running ends of synthesized molecules at the particular functionalized particle. Then, synthesis of the molecules of the first subset of molecules can continue from running ends incorporating the recognizable element, followed by capping (e.g., after final detritylation for dT molecules). Synthesis of molecules of the second subset of molecules can continue from running ends incorporating the protecting group, after deprotection of the protecting group (e.g., removal of the dA-Lev group).

Ratios of the first molecule type to the second molecule type at a functionalized particle can be: 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, or 1:99.

In variations where multiple types of molecules are coupled to the same functionalized particles, only a subset of molecules can be configured to be cleavable from the functionalized particle (e.g., depending upon intended assay).

Further embodiments, variations, and examples of the capture segment 141 can be structured as described in U.S. application Ser. No. 17/376,396 filed on 15 Jul. 2021, as incorporated by reference above.

The barcode segment 142 functions to enable identification of the functionalized particle with which it is associated, upon sequencing of the barcode segment 142. In some variations, the barcode segment 142 and/or other variations of the barcode segment 142 can function to enable identification of the substrate 110, entire distribution of functionalized particles 120 associated with the substrate 110, and/or position of a functionalized particle (and thus captured target analyte), upon sequencing. As such, readout of the barcode segment 142 can facilitate characterization of the distribution of target analytes at the substrate 110 and/or other aspects of the substrate 110 and distribution of functionalized particles 120.

The barcode segment 142 is preferably configured to be unique to each functionalized particle. Furthermore, the barcode segments 142 are configured to have diversity such that each functionalized particle or group of functionalized particles associated with the substrate 110 can be uniquely identified. In particular, the diversity of the bead barcode library can be at least 10-fold more than the number of functionalized particles deposited at the substrate, such that almost every functionalized particle has unique barcode on the substrate. Alternatively, the barcode segment 142 can be characterized in terms of diversity in another suitable manner. In embodiments, the barcode segment 142 can have from 5-50 bases in order to provide a sufficient number of unique sequences for a desired number of particles in solution for a given process (i.e., such that each particle can be uniquely identified); however, in alternative variations, the barcode segment 142 can have other suitable numbers of bases (e.g., less than 5 bases, more than 50 bases). Additionally or alternatively, the barcode segment 142 can have a number of bases designed to occupy a percentage (e.g., 10%, 20%, 30%, etc.) of the length of a unit of a respective molecule of a functionalized particle.

Furthermore, in some variations, a percentage of functionalized particles can have known barcode sequences and can be spiked in amongst the distribution of functionalized particles to serve quality control functions for downstream sequencing operations (e.g., in situ sequencing operations).

In use, decoding of the barcode/associated location can be performed using an optical approach (e.g., sequencing-by-synthesis with or without reversible terminators, in-situ sequencing that is ligation based, hybridization based, rolling circle amplification-based, fluorescence in situ hybridization-based, etc.), using a nearest-neighbor approach with a next generation sequencing readout (as described in the applications incorporated by reference), using morphology of the functionalized particles (e.g., using a pattern etched on the particle or other uniquely identifiable particle morphological feature, each feature associated with one barcode), using a combination of fluorescent colors emitted from the functionalized particle (e.g., each combination of fluorescent colors corresponding to a barcode), and/or using another suitable method.

In examples, decoding of the barcode/associated location can include performing a sequencing by ligation (SOLID) operation to decode nucleic acid labels (e.g., with sequencing primer sites, with UP primer sites, etc.) and positions of associate labels on functionalized particles at the substrate. Sequencing/decoding can be performed using a microfluidic device having a flow cell by which units the system 100 are processed for decoding prior to packing and delivery (e.g., in series, in parallel). The microfluidic device can thus control flow of materials for interactions with units of the substrate, in coordination with an imaging system having a field of view encompassing the substrate(s) configured to capture images from which sequences of the barcodes can be determined in relation to substrate positions.

The optional first cleavable linker 143 is coupled to the body 130 and functions to provide a mechanism by which molecules coupled to the body 130 can be controllably released from the body 130 (e.g., post-interaction with target analytes, or for controlled release to tag targets of a sample, such as nuclei targets). The first cleavable linker 143 can also extend units of the one or more molecules 140 out into space, thereby enabling interactions of the one or more molecules 140 with target analytes of the sample.

In embodiments, the optional first cleavable linker 143 is configured for selectable attachment (e.g., with functional groups specific to specific chemistries) and/or activatable cleavage, to enable controlled release of the one or more molecules 140 from the body 130, and/or controllable release of material derived from captured target analytes for downstream processing. In variations, activatable cleavage or separation in another manner can be achieved with linker regions configured to be cleave in response to one or more of: a thermal cleavage mechanism, a pH shift, a photocleaving mechanism, an enzymatic cleaving mechanism (e.g., as in molecular scissors), separation based upon changes in charge, or another suitable cleaving mechanism.

In variations, photocleaving can be achieved with photocleavable linkers that controllably cleave with specific light characteristics (e.g., wavelengths, intensities, exposure times, etc.). In such variations, controlled cleavage characteristics can prevent undesired cleavage of molecules (e.g., prior to use, such as in storage environments with ambient or other forms of light). In examples, photocleavable linkers can be structured to cleave with exposure to UV light (e.g., from 100-400 nm in wavelength), with an exposure time of 1 second, 2 seconds, 3 seconds, 4 seconds, 5 seconds, 10 seconds, 30 seconds, 100 seconds, 200 seconds, 300 seconds, or greater.

In composition, the optional first cleavable linker 143 is preferably composed of a polymer (e.g., non-nucleic acid polymer), and in a specific example, the first cleavable linker 143 can be composed of polyethylene glycol (PEG) or another suitable polymer. However, the first cleavable linker 143 can be composed of another suitable material (e.g., natural material, synthetic material).

In structure, the optional first cleavable linker 143 can have a linear structure that extends units of the one or more molecules 140 into space. Alternatively, the first cleavable linker 143 can have a branched or otherwise non-linear structure (e.g., dendrimer segment, dual-linking segment, other branched segment). For instance, in variations in which the first cleavable linker 143 is configured to control spacing/density of molecules coupled to the body 130, the first cleavable linker 143 can have a branched structure that reduces density and/or controls spacing/orientation of molecules coupled to the body 130. Additionally or alternatively, the length and/or structure of the optional first cleavable linker 143 can be configured to prevent steric hindrance of any enzyme or material that would interact with the oligonucleotide molecules during use. Exemplary dual-linker structures can have a free OH group on a 5′ end of the structure, or a phosphoramidite on a 5′ end of the structure to improve loading density characteristics. Branched structures can include protecting groups (e.g., lev protecting groups).

In relation to properties, the first cleavable linker 143 can be configured with a desired charge and/or other characteristic (e.g., level of hydrophilicity, level of hydrophobicity, etc.) that prevents undesired interactions between molecules (e.g., tangling, clumping, undesired structures, etc.). As such, the first cleavable linker 143 can be configured to extend molecules of the one or more molecules 140 into space (e.g., perpendicular from a surface of the body 130); however, the first cleavable linker 143 can be configured to extend from the body 130 in another suitable manner.

In variations, units of the one or more molecules can omit or include additional segments as needed. For instance, one or more of the one or more molecules 140 can include segments configured for amplification reactions (e.g., PCR handles, etc.). Additionally or alternatively, one or more of the one or more molecules 140 can include fluorescence-embedded labels. Additionally or alternatively, one or more of the one or more of the one or more molecules can include segments configured to simplify library preparation steps or sequencing processes of specific sequencing platforms. In more detail, molecules of the one or more molecules can include adapter segments (e.g., associated with P5/P7 adapters for Illumina™ platforms), index sequences associated with adapters, and/or other sequences. Additionally or alternatively, additional segments can be added during sample processing (e.g., during reverse transcription, etc.). Units of the one or more molecules 140 can additionally or alternatively include other sequences (e.g., for other fragmentation/sequencing/processing platforms). In embodiments, the molecules can be produced/synthesized by at least one of split pool synthesis (e.g., chemically, with an oligo synthesizer, etc.), enzymatic synthesis (e.g., with ligation and polymerase extension), by emulsion PCR, by template-free synthesis (e.g., using terminal transferase, etc.), and/or by other suitable methods, as described in more detail below.

In relation to capturing of different targets (e.g., cytoplasmic targets, nuclei targets, protein targets, targets from other sample regions, etc.)

Functionalized particles can have molecules with different PCR handles corresponding to different target types, in order to enable selective amplification, detection, and mapping of different target types.

Functionalized particles can have different subsets of molecules, where a first subset of molecules can be cleaved (e.g., in order to access a first target type such a nuclei target) by a first mechanism, and a second subset of molecules may not be cleavable (or cleavable by a second mechanism different than the first mechanism), in order to capture a second target type (e.g., a cytoplasmic target). The first cleaving mechanism or the second cleaving mechanism can include: a thermal cleavage mechanism, a pH shift, a photocleaving mechanism, a chemical cleaving mechanism, an enzymatic cleaving mechanism (e.g., as in molecular scissors), separation based upon changes in charge (e.g., as in an electrostatic interaction), or another suitable cleaving/separation mechanism. In relation to cleaving, samples or processing reagents can include diffusion limiting compounds (e.g., long-chain polymers, such as in polyethylene glycol solutions), in order to prevent diffusion of cleaved functionalized particles beyond a threshold distance away from originating positions. Diffusion limiting compounds can thus enable accurate mapping of targets, even when cleavable capture molecules are implemented to capture targets post cleaving. In use cases where a nuclei target is tagged with functionalized molecules originating from different functionalized particles (e.g., as determinable using barcode sequences or other spatial tag sequences), bioinformatics can be used to characterize locations of such nuclei targets, as well as diffusion behavior of cleaved molecules (e.g., average distances diffused in order to arrive at a target), which can be used to improve SNR of maps generated.

For instance, in embodiments where a nucleus is tagged using cleavable molecules of functionalized particles, tagging of the nucleus can involve tagging the nucleus with different cleavable molecules associated with different spatial positions. A bioinformatics approach can involve determining the position of the nucleus based upon a subset of positions corresponding to a subset of stochastic barcodes of molecules that tagged the nucleus. The position of the nucleus can be determined from an average position of the subset of stochastic barcodes (e.g., a centroid of positions of the subset of stochastic barcodes). In variations, nuclei can be tagged using a combination of cleavable and non-cleavable molecules, such that positions of the nuclei can be determined from stochastic/spatial barcode positions of cleavable and non-cleavable molecules (e.g., as a weighted centroid of positions, where positions of non-cleavable components are weighted more heavily than positions of cleavable components).

Different molecules configured to capture different targets can be coupled to the same particle/feature/body. Alternatively, different subpopulations of particles/features/bodies can be paired with different molecules configured to capture different targets.

Molecules can be coupled to functionalized particles with a suitable density of molecules per particle. For instance, a particle can have on the order of 10s to 100s of molecules for mapping nuclei targets and/or other target types.

Additionally or alternatively, molecules can be coupled to functionalized particles with a suitable percentage of surface area covered by molecules, where the percentage can be 5%, 10%, 15%, 20%, 25%, 30%, 35%, 40%, 45%, 50%, 55%, or another suitable percent coverage. Percent coverage, in addition to other sample processing aspects described, can improve recovery rate of nuclei targets mapped (e.g., in relation to actual numbers of nuclei targets present) to greater than 15%, greater than 20%, greater than 25%, greater than 30%, greater than 35%, greater than 40%, greater than 45%, greater than 50%, or greater.

Some embodiments, variations, and examples of molecule segments are further described in U.S. application Ser. No. 17/376,396 filed on 15 Jul. 2021, which is incorporated by reference above.

### 2.3 System-Support Structure and Use

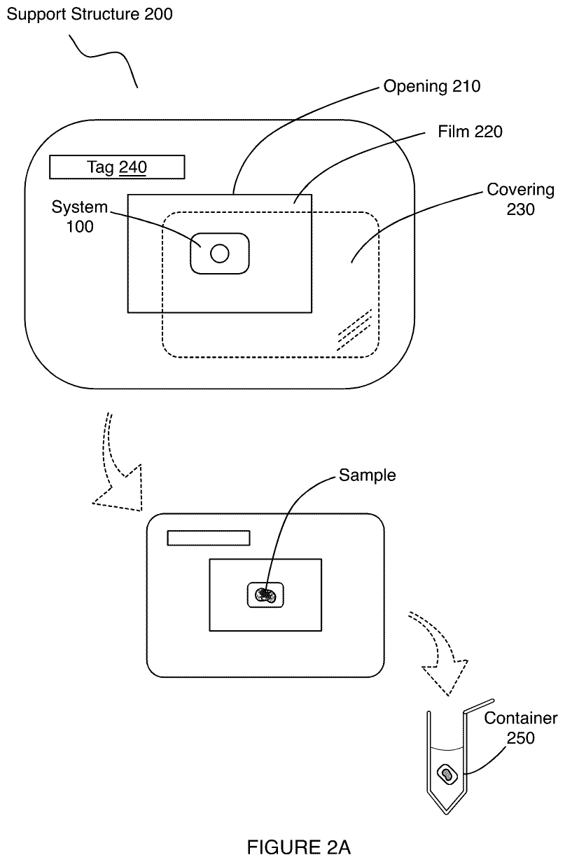

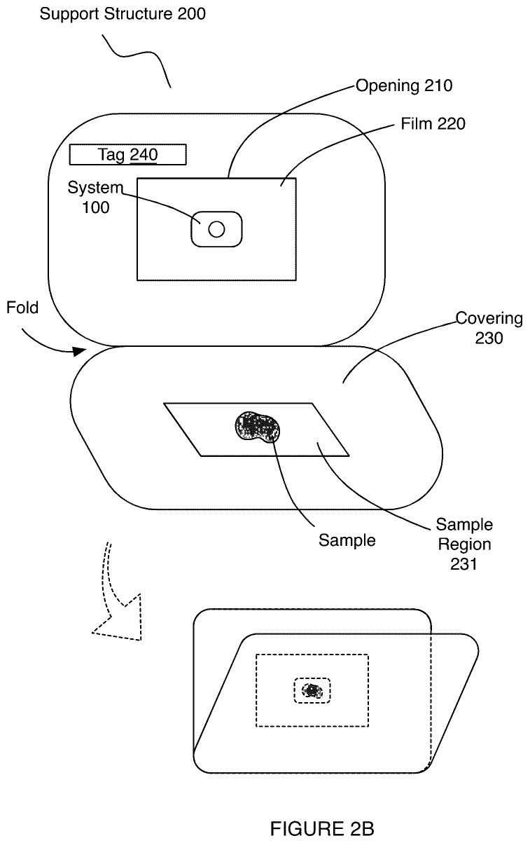

As shown in FIG. 2, the invention(s) can further include a support structure 200, which functions to support one or more units of the system 100 and to facilitate usage of the system 100 by a sample-processing entity. In embodiments, the support structure 200 can retain one or more units of the system 100 in position, facilitate contacting of units of the system 100 by a tissue sample or other sample, and enable release of the one or more units of the system 100 from the support structure 200 for further processing, post-interactions with sample material.

In embodiments, as shown in FIGS. 2A and 2B, the support structure 200 includes: an opening 210, and a flexible film 220 coupled to the support structure about the opening 210 at a first surface of the support structure, and supporting one or more units of the system 100 (e.g., substrate 110 with functionalized particles 120, array of substrates with functionalized particles, etc.) within the opening 210. In variations, the support structure 200 can further include one or more of: a protective covering 230 opposing the flexible film 220 at the opening 210, and a tag 240 encoding information associated with the system 100/substrate 110.

Details of embodiments, variations, and examples of the support structure 200 and related structural components are further described as follows:

In embodiments, the support structure 200 is composed of a material that provides protection from environments associated with transportation and/or use of the system 100. As such, the support structure 200 can function to provide one or more of: impact resistance (e.g., to protect fragile portions of the system 100), flame-proofing/resistance; sealing (e.g., as in a hermetic seal, in order to prevent intrusion of moisture prior to use, in order to prevent intrusion of gases prior to use, etc.); optical properties (e.g., with respect to shielding from electromagnetic energy, with respect to allowing transmission of light for enabling optical detection or visual observation through the support structure 200, etc.); electrical properties (e.g., with respect to shielding from electric fields, etc.); and/or other suitable properties. In examples, the support structure 200 can be composed of a polymer material, a fibrous material, a foam, or another suitable material.

In morphology, the support structure 200 can have a broad surface, where the broad surface is rectangular in morphology; however, in other variations, the support structure 200 can have another suitable defined morphology (e.g., circular morphology, ellipsoidal morphology, polygonal morphology, amorphous morphology, etc.). In examples, the support structure 200 can have a characteristic length from 1-15 centimeters, a characteristic width from 1-15 centimeters, and a characteristic thickness from 0.5-5 millimeters; however, the support structure 200 can alternatively have other suitable dimensions.

In variations, the support structure 200 can include features configured to facilitate handling by an operator. For instance, the support structure 200 can include surface regions with high-friction (e.g., to facilitate gripping by a human or robotic operator), markings (e.g., to provide indications of orientation, to guide proper usage of the support structure 200 in relation to various operation modes, etc.), and/or other features.

The opening 210 functions to enable an operator to interact with the system 100 according to operation modes described below, when the system 100 is supported by the support structure 200. In particular, the opening 210 can allow the operator to transmit heat to a sample (e.g., through the flexible film 220 described in more detail below) and/or to displace the system 100 from the support structure 200 (e.g., by application of a mechanical force to the flexible film 220 described in more detail below). The opening 210 can be rectangular in morphology; however, in other variations, the opening can have another suitable defined morphology (e.g., circular morphology, ellipsoidal morphology, polygonal morphology, amorphous morphology, etc.). In examples, the opening 210 can have a characteristic length from 0.2-10 centimeters, a characteristic width from 0.2-10 centimeters, and a characteristic depth from 0.5-5 millimeters; however, the support structure 200 can alternatively have other suitable dimensions.

The support structure 200 can include a single opening 210. Alternatively, the support structure 200 can include a set of openings (e.g., arranged as an array, arranged in another suitable manner), where each opening of the set of openings is configured to support one or more units of the system 100.

The flexible film 220 functions to support the system 100 within the opening 210 (or multiple openings), and to enable operation modes described in more detail below. Properties of the flexible film 220 can allow an operator to transmit heat to a sample (e.g., through the flexible film 220 and/or substrate 110) and/or to displace the system 100 from the support structure 200 (e.g., by application of a mechanical force to the flexible film 220).

In embodiments, the flexible film 220 is retained in position about the opening 210 (e.g., coupled to a broad surface of the support structure 210, retained between layers of the support structure 210, etc.). The flexible film 220 can be retained without any slack or under tension. Preferably, the flexible film 220 is retained in a manner such that application of force or heat to the flexible film 220, as intended during use during operation modes described below, does not compromise coupling of the flexible film 220 to the support structure 200 or compromise its functionality. Alternatively, the flexible film 220 can be retained in position about the opening 210 in another suitable manner.

The flexible film 220 is preferably composed of a material processed with: suitable mechanical properties (e.g., in relation to tear strength, in relation to strain behavior, in relation to allowing plastic deformation for displacement of the system 100 from the support structure 200, in relation to allowing elastic deformation for displacement of the system 100 from the support structure 200, etc.), optical properties (e.g., degree of transparency to allow observation of a unit of the system 100 coupled to the flexible film 220 during use), thermal properties (e.g., in relation to conductivity for transmission of heat to a sample through the flexible film 220 and substrate 110, in relation to melting temperature, etc.), surface and bulk properties (e.g., in relation to charge, in relation to degree of hydrophobicity, in relation to porosity, etc.), electrical properties, and/or other suitable properties. The flexible film 120 can have a thickness from 75 to 150 micrometers (or alternatively, another suitable thickness).

In embodiments, the flexible film 120 is a flexible polymer film composed of polyvinyl chloride (PVC), polyolefin, polyethylene, polyethylene terephthalate (PET), nylon, and/or another suitable polymer material. In variations, the flexible film 120 further includes an adhesive layer coupled thereto, in order to provide a mechanism for coupling with units of the system 100 in a non-permanent manner. In variations, the adhesive layer is composed of an acrylic adhesive; however variations of the adhesive layer can be composed of another suitable material. The adhesive layer can have an adhesive strength configured based upon size and mass characteristics of the system 100 and/or in relation to specified force required to displace a unit of the system 100 from the flexible film 120 during use. In a specific example, the flexible film 120 is a PVC dicing tape used during manufacturing of the substrate 110 (e.g., with respect to scribing and sawing of the substrate 110); however, in variations of the specific example, the flexible film 120 can be otherwise composed and configured.

For instance, as an alternative to mechanical breaking of adhesive bonds, the adhesive layer can be structured such that adhesive bonds are broken upon exposure to specific wavelength ranges of light (e.g., ultraviolet light, etc.), thermal stimulation (e.g., upon exposure to heat at certain temperature ranges), and/or another suitable mechanism.

The protective covering 230 functions to protect units of the system 100 supported by the support structure 200 (e.g., during transportation, during phases of use, etc.). In some embodiments the protective covering 230 is composed of the same material as the bulk material used for the support structure 200; however, the protective covering can alternatively be composed of another suitable material, embodiments, variations, and examples of which are described above.

The protective covering 230 can be an element separate from the bulk material of the support structure 200, such that the protective covering 230 can be provided with the support structure 200 (e.g., in relation to the kit/package described in more detail below), and removed from the support structure 200 during use. Alternatively, the protective covering 230 can be physically contiguous with the bulk material of the support structure 200 and/or transitionable between a covered mode (e.g., in which units of the system 100 are covered) and an uncovered mode (e.g., in which units of the system 100 are uncovered), where transition between modes can be enabled through folding, sliding, or another mechanism facilitated by structural relationships between the protective covering 230 and the bulk material of the support structure 200. In one such variation, as shown in FIG. 2B, the protective covering 230 can be folded over the opening 210 in the covered mode, and unfolded in the uncovered mode.

Sample retention and positioning for interactions with the system 100 can, however, be enabled by other suitable sample positioning structures.

Furthermore, in relation to operation modes described in more detail below, the protective covering 230 can include a sample region 231 configured to support or retain a sample, as shown in FIG. 2B, where the sample can be positioned at the sample region 231, and then transition of the protective covering 230 to the covered mode (e.g., through folding, through another mechanism) can position the sample into contact with the system 100 for sample processing in a consistent and reliable manner.

The tag 240 functions to encode information pertaining to one or more of the support structure 200, units of the system 100 supported by the support structure 200, reagents being provided with a kit including the support structure 200, the sample(s) being processed using the support structure 200, molecular barcode information (e.g., spatial locations associated with different molecular barcodes of distributions of functionalized particles 120, etc.). In variations, information encoded by the tag 240 can include one or more of: batch number (e.g., of the support structure 200, of a system 100 unit, of reagents), lot number (e.g., of the support structure 200, of a system 100 unit, of reagents), sample-identification information, patient or subject information associated with a sample, molecular barcode information (e.g., spatial locations associated with different molecular barcodes of distributions of functionalized particles 120, etc.), other spatial information (e.g., associated with a position of a system 100 unit at the support structure, associated with spatial locations of material at the system 100 unit), other molecular information, and/or other suitable information.

In embodiments, the tag 240 can be a computer-readable tag. In embodiments, the tag 240 can thus have the form of a barcode, a QR code, a code including characters (e.g., alpha-numeric characters, other characters, etc.), or another suitable code that is readable upon scanning (e.g., with an optical detection subsystem). Additionally or alternatively, the tag 240 can be a digitally-readable tag (e.g., decoded upon transmission of electrical signals).

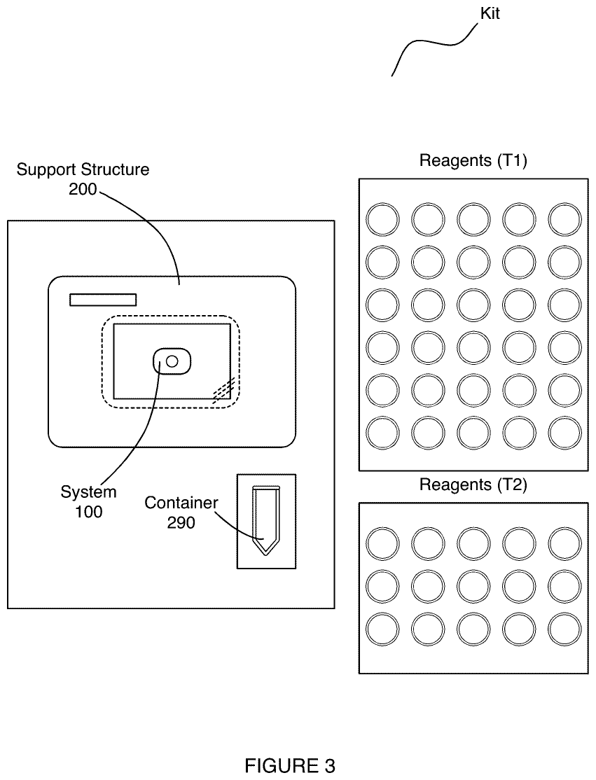

In embodiments, the support structure 200 with one or more units of the system 100 can be provided as a kit (as shown in FIG. 3), where the support structure 200 is assembled (e.g., pre-packaged) with the one or more units of the system 100, including the substrate 110 with the distribution of functionalized particles 120. The kit can further include a process container 250 configured to receive one or more units of the system 100 (e.g., in relation to operation modes described below where the system 100 is displaced from the support structure into a container 250, such as a process container or collecting container).

Additionally or alternatively, the kit can further include one or more reagents for sample processing operations and/or for library preparation operations. One or more of the reagents can be provided in separate containers; additionally or alternatively, one or more of the reagents can be provided in container 250 (e.g., pre-packaged in container 250), in order to stabilize or store material captured at a unit of the system 100 (e.g. prior to transportation or other downstream processing operations).

In one variation, provided reagents can be designed for reception/storage of the system 100 at a first temperature (e.g., −20 C). In examples, the reagents can include one or more of: RNase inhibitor, superscript/reverse transcriptase buffer, reverse transcriptase enzyme, dNTPs, reverse transcription enzyme, template switching oligonucleotides, exosome isolation reagents, TC enzyme/buffer, superscript enzyme, amplification primers, PCR reagents (e.g., PCR buffer, PCR primer mix, PCR enzyme, etc.), proteinase K, exonuclease, cDNA amplification buffer, cDNA amplification primer mix(es), cDNA amplification enzyme, TE, and/or other suitable reagents.

In another variation, provided reagents can be designed for reception/storage of the system 100 at a second temperature (e.g., 4 C, etc.). In examples, the reagents can include one or more of: functionalized particle washing buffer, TC enzyme/buffer, water (e.g., nuclease-free water), hybridization buffer, tissue clearing buffer, Tris buffer, sodium hydroxide, and/or other suitable reagents.

In another variation, provided reagents can be designed for reception/storage of the system 100 at a third temperature (e.g., room temperature, etc.). In examples, the reagents can include one or more of: functionalized particle washing buffer, TC enzyme/buffer, water (e.g., nuclease-free water), hybridization buffer, and/or other suitable reagents.

Additionally or alternatively, reagents can be configured for library preparation and/or other assays. In examples, library preparation materials can support hybridization (e.g., hybridization with whole genome sequencing primer sites, with universal primer (UP) sites, etc.), template switching reverse transcription (RT), sample and bead removal (e.g., within process container 250), exonuclease treatment or other methods of removing single stranded oligonucleotides from functionalized particles, denaturation steps (e.g., involving sodium hydroxide), second strand synthesis

Reagents of the kit can be provided in a separate housing (e.g., container, box, etc.) from the support structure 200 and/or other system elements, examples of which are shown in FIG. 3. Additionally or alternatively, reagents of the kit can be provided in the same housing (e.g., container, box, etc.). Additionally or alternatively, the kit can include open receptacles (e.g., for optional or custom reagents), or can otherwise omit reagent provision.

Additionally or alternatively, the kit can include training substrates (e.g., substrates with or without functionalized particles, and/or with or without decoding of functionalized particle positions at the substrate), which can be used by new users to practice application of a sample to the substrate and/or to practice other aspects of using the kit.

Other examples of the kit can include: one or more units of a substrate with functionalized particles for tagging nuclei of a sample (embodiments, variations, and examples of which are described above), a set of reagents (e.g., dissociation buffers, extraction buffers, wash buffers, RNase inhibitors, etc.) for nuclei isolation of a sample, and/or other components.

In a first example, a substrate of a kit can include a first subpopulation of bodies having a first diameter (e.g., a diameter of 10 microns, a diameter of 3 microns) and/or other first characteristic, and a second subpopulation of bodies having a second diameter (e.g., a diameter of 10 microns, a diameter of 3 microns) and/or other second characteristic. In the first example, bodies of the first subpopulation can be functionalized (e.g., with a first capture molecule type) for capturing a first target type (e.g., cytoplasmic targets, sample surface targets, mRNAs by polyA/polyT interactions, etc.). Bodies of the second subpopulation can be functionalized (e.g., with a first capture molecule type) for capturing a second target type (e.g., targets comprising nuclei or nuclei-associated targets, for instance, with randomer segments, segments that are gene-specific to nuclei targets, segments that capture extended nuclei targets, segments that capture polyadenylated nuclei targets, or other capture segments for nuclei targets). Bodies of the first subpopulation and the second population can be randomly or non-randomly distributed across the active region of the substrate.

In a second example, a substrate of a kit can include a first subpopulation of bodies having a first diameter (e.g., a diameter of 10 microns) and/or other first characteristic, and a second subpopulation of bodies having a second diameter (e.g., a diameter of 3 microns) and/or other second characteristic. In the first example, bodies of the first subpopulation can be functionalized (e.g., with a first capture molecule type) for capturing a first target type (e.g., cytoplasmic targets, sample surface targets, mRNAs by poly A/polyT interactions, etc.). Bodies of the second subpopulation can be functionalized (e.g., with a first capture molecule type) for capturing a second target type (e.g., targets comprising nuclei or nuclei-associated targets, for instance, with randomer segments, segments that are gene-specific to nuclei targets, segments that capture extended nuclei targets, segments that capture polyadenylated nuclei targets, or other capture segments for nuclei targets). Bodies of the second subpopulation can be dispersed interstitially (e.g., in spaces between) or amongst the first population at a substrate without compromise of mapping resolution for all target types. As such, in this application, multiple subpopulations of functionalized particles can be implemented in order to capture different targets simultaneously, for characterizations of distributions of different targets using the same system.

In a third example, a substrate of a kit can include bodies with a first subset of molecules functionalized (e.g., with a first capture molecule type) for capturing a first target type (e.g., cytoplasmic targets, sample surface targets, mRNAs by polyA/polyT interactions, etc.) and a second subset of molecules functionalized (e.g., with a first capture molecule type) for capturing a second target type (e.g., targets comprising nuclei or nuclei-associated targets, for instance, with randomer segments, segments that are gene-specific to nuclei targets, segments that capture extended nuclei targets, segments that capture polyadenylated nuclei targets, or other capture segments for nuclei targets). An example is shown in FIG. 1D with respect to particles of a 2nd particle type.

A workflow associated with the first, the second, and the third examples of the kit can involve: receiving a sample at the substrate with the distribution of functionalized particles; performing a hybridization reaction with capture portions of functionalized molecules of the functionalized particles with targets of a first target type (e.g., nuclei targets) and targets of a second target type (e.g., cytoplasmic targets) of the sample; performing a reverse transcription operation upon products of the hybridization reaction; performing a tissue dissociation operation and a nuclei operation; and performing sequencing operations for generating spatial maps of the first target type and the second target type across the sample.

### 2.3.1 Support Structure—Example Operation and Variations

During use, the support structure 200 can provide a set of operation modes for sample processing (e.g., with respect to protecting aspects of the system 100 during shipping/handling/processing, with respect promoting contact between units of the system 100 and samples during processing, with respect to enabling release of units of the system 100 from the support structure 200 for downstream processing, etc.).

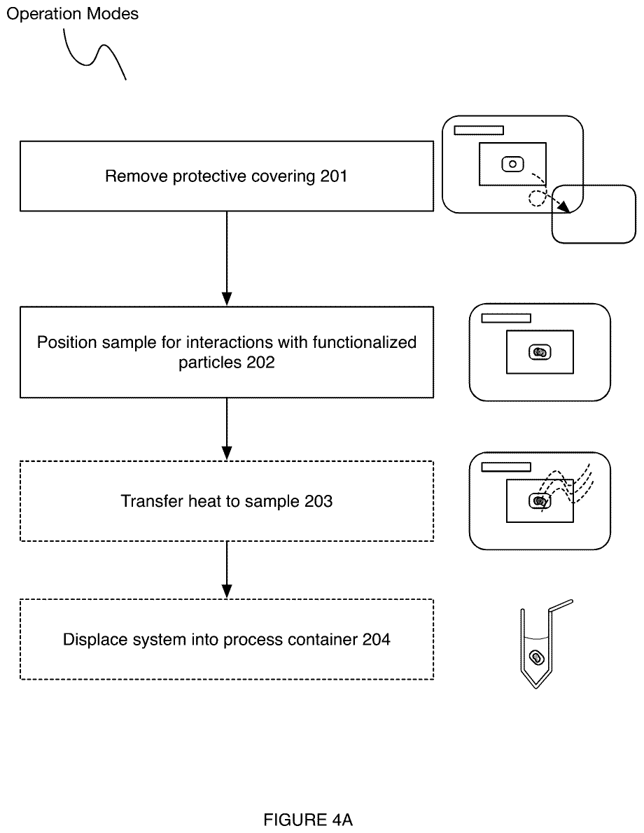

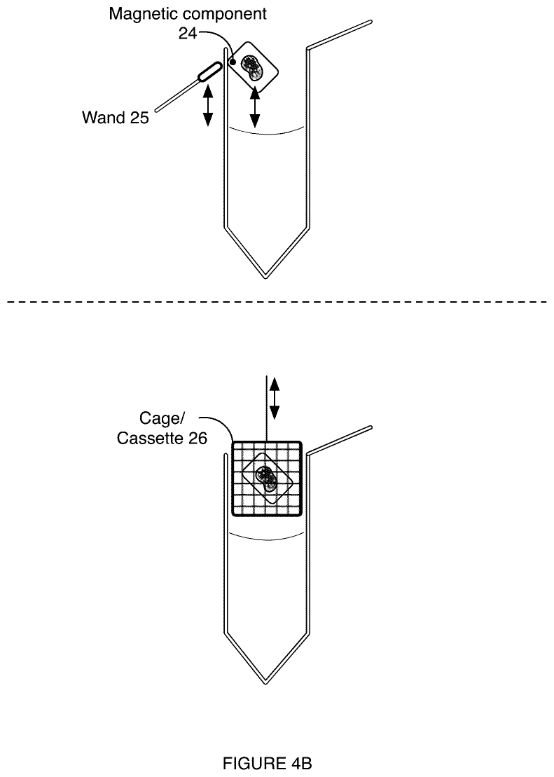

In variations, as shown in FIG. 4A, the set of operation modes can include a first operation mode 201 in which the protective covering 230 is removed from the support structure 200 or otherwise positioned away from the substrate 110; and a second operation mode 202 in which a sample is positioned in contact with the substrate 110 and the distribution of functionalized particles 120. The set of operation modes can further include a third operation mode 203 in which heat is transferred from the substrate 110 to the sample (e.g., in variations in which the sample is frozen, in variations in which the sample is paraffin-embedded, etc.); and a fourth operation mode in which the flexible film 220 is deformed, thereby displacing the substrate 110, with or without the sample, from the support structure 200 and into a process container 250. Additionally or alternatively, additional operation modes can include facilitating removal of functionalized particles from the substrate 110, within the process container 250 (e.g., by aspiration and delivery of liquid within the process container 250 to dislodge functionalized particles from the substrate 110 after they have interacted with the sample).

As described above, the first operation mode 201 and the second operation mode 202 can be associated with covered and uncovered modes provided by the protective covering 230, where the protective covering 230 can receive the sample, and transition to the covered mode (e.g., by folding), in order to position the sample into contact with the distribution of functionalized particles 120 at the substrate 110. Operation modes 201 and 202 can be enabled by variations of the support structure 200 in another suitable manner (an example of which is described in relation to variation of the support structure 200b below).

In relation to operation mode 203, heat can be transferred from the substrate 110 to the sample through the flexible film 220. In a first example, an operator can position a warm object (e.g., finger, heating element, etc.) against the flexible film 220 opposite the system 100, and heat from the warm object can be transmitted to the sample (e.g., to melt the sample). In another example, a platform (e.g., automated platform) can transmit heat to the sample with a heat source (e.g., plate heater, convective heater, etc.) in thermal communication with the sample (e.g., through the flexible film, through the support structure, through the substrate, etc.). In particular, in relation to operation modes described, at least one of the flexible film 220, the substrate 110, and the support structure 100 has a thermal conductivity of greater than a thermal conductivity threshold (e.g., 0.05 W/mK), providing a thermal transmission pathway to the sample during operation.

In relation to operation mode 204, the flexible film 220 is deformed, to displace the substrate 110 from the support structure 200 and into a process container 250 for transportation, storage and/or further processing (e.g., sequencing, etc.). In one variation, an operator can apply a force to the flexible film 220 (e.g., backside of the flexible film 200), to displace the substrate 110 from an adhesive layer coupled to the flexible film 220. In another variation, a robotic apparatus can apply a force (e.g., using a tip or other extremity) to the flexible film 220 (e.g., backside of the flexible film 200), to displace the substrate 110 from an adhesive layer coupled to the flexible film 220. In alternative variations, operation mode 204 can omit implementation of a mechanical force, and instead application of light within a specified wavelength range (e.g., UV light) and/or application of heat (e.g., at a specified temperature range) can promote separation of the substrate 110 from the film 220.

Additionally or alternatively, in relation to operation mode 204, the system can include a magnetic component 24 (e.g.) coupled to the substrate 110, or to which the substrate 110 with the distribution of functionalized particles is transferred, prior to transfer of the substrate 110 into the process container, as shown in FIG. 4B (Top). Furthermore, the system 100 or other entity performing sample processing can apply magnetic forces (e.g., by actuating a magnetic wand/stylus 25) to the substrate 110, in order to control motion of the substrate 110 into, out of, or within the process container 250 during sample processing steps.

Additionally or alternatively, in relation to operation mode 204, the system can include a cage/cassette 26 into which the substrate 110 with the distribution of functionalized particles is transferred in coordination with interacting the distribution of functionalized particles with the sample, where the cage/cassette can be manipulated more easily than the substrate, for controlling motion of the substrate 110 into, out of, or within the process container 250 during sample processing steps, as shown in FIG. 4B (Bottom).

In variations the film may not be flexible and can instead be rigid.

The invention(s) described can support further operation modes and/or include other elements. For instance, the invention(s) can include a flow cell configured to receive one or more units of the system 100 (e.g., post-interaction with samples, and post-displacement from a support structure), where the flow cell enables sequencing of target analytes, material derived from captured and processed target analytes, and/or other sample processing steps. Such a flow cell can thus include a fluid channel in communication with the distribution of functionalized particles at the substrate of a unit of the system, and enable optical detection of signals generated from captured and/or processed target analytes of the sample. Furthermore, the flow cell can enclose one or more units of the system for higher throughput and/or multiplexed operations.

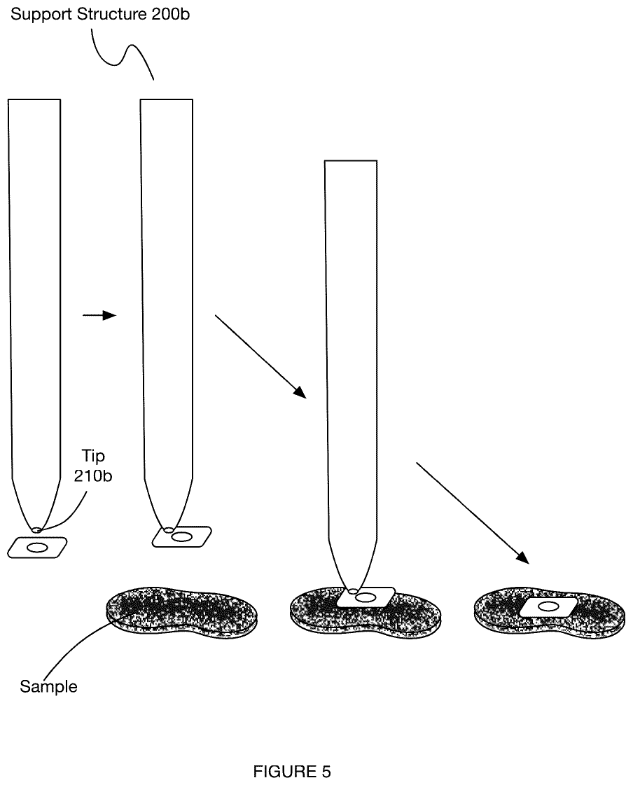

Alternative Variation: In an alternative variation, as shown in FIG. 5, the support structure 200b can include a tip 210b supporting one or more units of the system 100, where, during use, the tip 210b can be positioned into contact with a sample, and the unit(s) of the system 100 can be displaced from the tip 210b to contact a specific portion of the sample. In examples, the unit(s) of the system 100 can be coupled to the tip 210b using a layer (e.g., adhesive layer), where contact with the sample provides a force that separates the system 100 from the layer to interact with the sample. Additionally or alternatively, the tip 210b and/or other portion of the support structure 200b can provide a controlled release mechanism using, for example, a plunger (e.g., mechanical plunger that displaces the system 100 from the tip 210b), magnetic forces (e.g., in which reversible polarity or removal of magnetic forces displaces the system 100 from the tip 210b), or other forces are used to separate the system 100 from the tip 210b.

Furthermore, the variation of the support structure 200b described can include multiple tips individually supporting units of the system 100, where the multiple tips can be synchronously controlled and/or individually controlled to displace respective units of the system 100 to promote sample interactions, target analyte capture, target analyte location characterization, and/or other aspects of sample processing.

In a specific application of use, a sample/tissue can still be integrated with a patient or other subject (e.g., not removed from the patient/subject), and in specific examples, the sample/tissue can include a skin sample (e.g., lesion) or other section of tissue made accessible (e.g., during a procedure for biopsy, during orthoscopy, during endoscopy, etc.), allowing sampling of the tissue without removal from the patient. For instance, in one specific use case, during removal of cancerous tissue, the support structure 200b can apply one or more system units to the neighboring tissue to help confirm if the cancerous tissue is completely removed. The support structure 200b can, however, be applied in other suitable manners.

Still other variations of the support structure can be otherwise configured with respect to promoting interactions with samples for target analyte capture, target analyte location characterization, and/or other aspects of sample processing. For instance, the system can include a sample positioning structure configured to retain the sample in position relative to the substrate.

### 3. Methods of Manufacturing

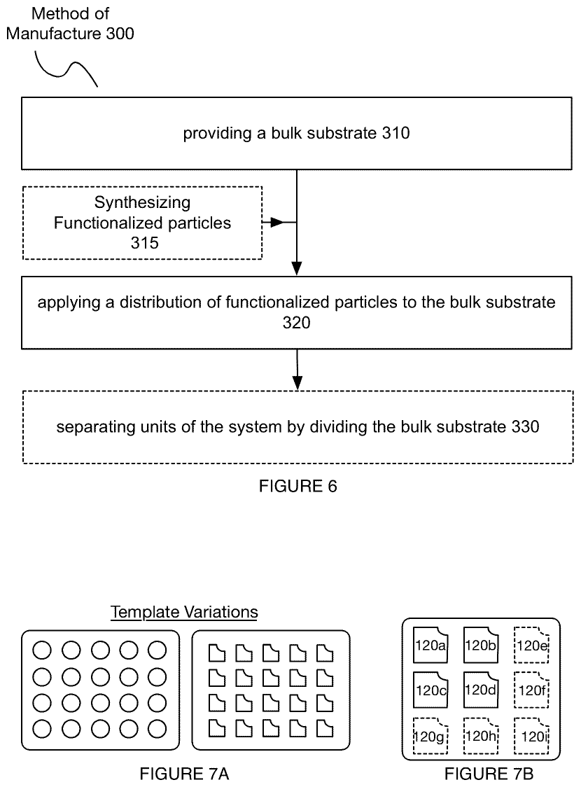

As shown in FIG. 6, a method 300 for manufacturing units of system embodiments described above includes: providing a substrate 310; and applying a distribution of functionalized particles to the substrate 320. Compositions of system components can be provided as described in the embodiments, variations, and examples covered in Section 2 above; however, compositions of system components can additionally or alternatively include other compositions.

Providing the substrate 310 can include providing a bulk substrate that can be separated into separate units (each having one or more distributions of functionalized particles). Separating the bulk substrate into separate units can implement fiducials that facilitate separation of the bulk substrate (e.g., manually, in an automated manner). In variations, the fiducial(s) can be coupled (e.g., etched into, applied with an adhesive, marked with an ink, chrome-marked, cut into, etc.) to the surface of the bulk substrate. Alternatively, the bulk substrate can be positioned adjacent to or retained in position adjacent to another substrate (e.g., sheet) having markings/fiducials during associated fabrication and separation operations. Additionally or alternatively, in relation to multiple distributions of functionalized particles on a substrate, each distribution can have associated fiducials for location identification purposes and/or other purposes

Applying a distribution of functionalized particles to the substrate 320 can involve a coating and/or deposition process. In variations, step 320 can include coating or depositing layer 112 described above onto the surface of the substrate prior to coupling of the distribution of functionalized particles to the substrate. In one example, coating layer 112 onto the substrate involves spin-coating the layer 112 onto the substrate in one or more stages, each stage involving de-gassing material of the layer 112 (in liquid form, if solvents are present), and spin-coating the de-gassed material onto the substrate (e.g., with a final layer thickness from 0.05 to 2 millimeters). The coating process can include multiple coats, desired spin rates, dynamic dispensing of coating material, static dispensing of coating material, and/or suitable drying processes (e.g., between 35 and 60 C, with heating during centrifugation, etc.). Additionally or alternatively, the layer 112 can be deposited using an ultrasonic deposition process and/or other process (described further in relation to manufacturing methods below). The coating process can further produce a desired level of roughness and/or texture, as described above.

Then, applying the distribution of functionalized particles onto the substrate involves dispensing the distribution of functionalized particles, in solution, into openings of a template aligned in position with the substrate, followed by centrifugation (e.g., with heating at 40 C, at another suitable temperature), and drying. As such, the functionalized particles are applied randomly to the substrate within openings of the template. Additionally or alternatively, the distribution of functionalized particles can be spin-coated, printed, or applied in another suitable manner (described further in relation to manufacturing methods below). The openings of the template can be sized according to characteristic dimensions described above, and/or have desired shapes and morphologies as described above. The openings of the template can further be spaced to facilitate separation of units of the system in downstream manufacturing steps. In variations (shown in FIG. 7A), the openings can be circular (e.g., 1.5-5 millimeters in diameter), with center-to-center spacing ranging from 1.75 to 10 millimeters. In an alternative variation (shown in FIG. 7A), the openings can be square (3 millimeters by 3 millimeters) with a notch for orientation, and center-to-center spacing of 4.5-5 millimeters. However, the template can be otherwise configured.

Applying the distribution of functionalized particles can further include centrifuging the substrate (e.g., at a desired temperature) with the solution of functionalized particles, washing the substrate (e.g., to remove stacked/overlapping particles, to produce a monolayer of particles, etc.), implementation of reducing reagents, implementation of recovery reagents, and/or other suitable processing steps. In examples, washing the distribution of functionalized particles can include pelleting the functionalized particles and washing the pellet(s) with a wash buffer. Alternatively, in other examples, washing the distribution of functionalized particles can include performing washing of functionalized particles within a column (e.g., chromatographic column), using a wash buffer. However, washing can be performed in another suitable manner.

The distribution of functionalized particles can additionally or alternatively be applied using another suitable deposition, coating (e.g., ultrasonic coating), or printing process.

In embodiments, the method 300 can further include synthesizing the functionalized particles 315 (e.g., prior to application of the distribution of functionalized particles to the substrate), where synthesis can include functionalizing the particle bodies with linker molecules (e.g., as described above), and performing one or more synthesis operations (e.g., split pool synthesis with an oligonucleotide synthesizer, enzymatic synthesis with ligation and polymerase extension, emulsion PCR, etc.), to produce resultant functional molecules for target capture and identification, at each particle. Synthesis can further include agitation steps (e.g., agitation of functionalized particles) to promote control/consistency of synthesis. Synthesis can further implement use of exonucleases to remove truncated oligonucleotides that could interfere with capture or amplification efficiency, as well as accurate barcoding. In alternative variations, synthesis can include functionalization for protein or amino acid-associated assays (e.g., with etching of antibodies, with implementation of oligonucleotides with poly DA and antibody-specific barcodes, etc.), and/or other suitable processing steps.

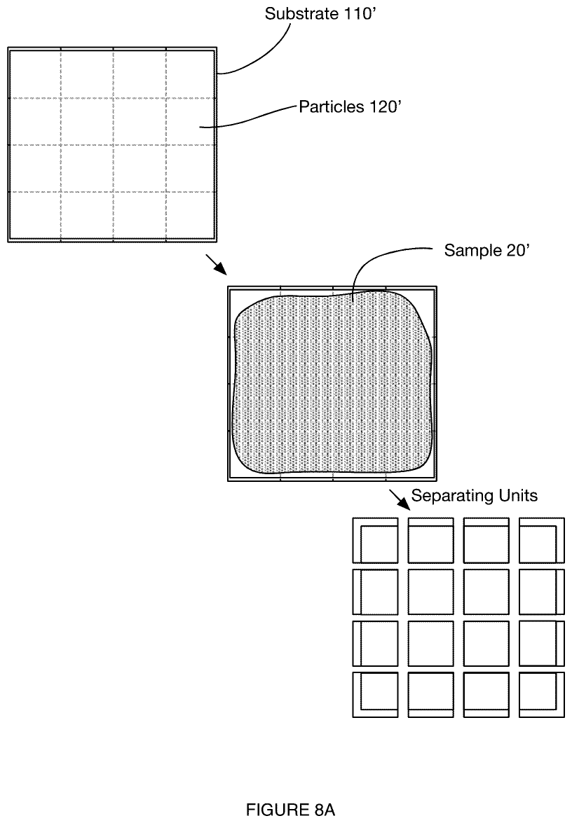

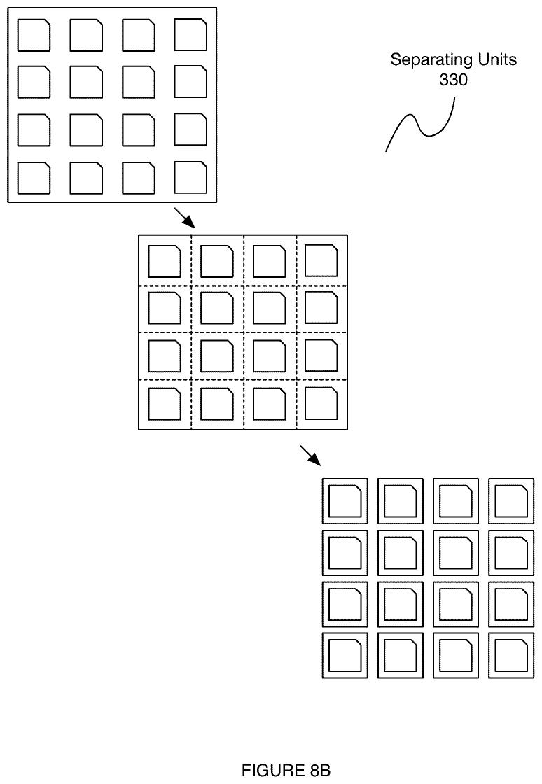

In some embodiments, the method 300 can further include separating units of the system from each other 330, which functions to enable scaling of manufacturing of system units in an efficient manner. In one variation, separating units can include scribing the bulk substrate between units of distributions of functionalized particles (e.g., using fiducials described above, using other alignment methods, etc.), and cleaving units from each other, according to the scribing pattern, as shown in FIG. 8. Additionally or alternatively, separating units can implement one or more of: etching, scoring, cutting (e.g., laser cutting), sawing, or another suitable separation method, with pick-up tools (e.g., vacuum tweezers, etc.) to relocate separated units for packaging. However, as described above, in relation to generating substrates with multiple units of distributions of particles, the bulk substrate can be separated in a manner that produces arrays of distributions of functionalized particle at each separated unit.

Additionally, the method 300 can further include generating documentation (e.g., text files, databases) of locations (e.g., in Cartesian coordinates, in cylindrical coordinates, in spherical coordinates, etc.) of functionalized particles and associated barcode sequences, as well as other information characterizing system, support structure, sample, and/or other aspects of the invention(s) described.

In specific examples, units of the system can be configured to fit within a collecting container (e.g., provided with kit described above), and be submerged or otherwise placed in contact with process fluids (e.g., for perfusion) in the collecting container. As such, in specific examples, separation of individual units of the bulk substrate can produce unit sizes having lengths from 3-10 millimeters and widths from 3-10 millimeters (e.g., with distances between units of 1 millimeter to 5 millimeters).

In variations, manufacturing and quality control (QC) operations associated with manufacturing and/or sequencing (e.g., with optimization to significantly reduce processing time) can include one or more of: assessing monolayer formation of functionalized particles (e.g., through image analysis, etc.); assessing smoothness or roughness of the coating of layer 112 (e.g., adhesive layer), such as through atomic force microscopy (AFM) or other suitable methods; detection and identification of spatial labels associated with target analytes by way of at least one of: in situ sequencing (e.g., ligation-based sequencing, hybridization-based sequencing, rolling circle amplification, other decoding operations, etc.) and detection of a spatial nucleic acid label associated with at least one of: a morphological feature of a functionalized particle and fluorescent compositions of particles during synthesis of the functionalized particle; operations to determine functionalized particle sensitivity; operations to improve focusing and image optimization associated with imaging subsystems used for sequencing (e.g., with magnification optimization to reduce number of images and/or image size); other operations to characterize capture efficiency (e.g., to produce greater than 90% recovery of oligonucleotides from functionalized particles, to produce greater than 85% recovery of oligonucleotides from functionalized particles, to produce other suitable capture efficiency rates, etc.); operations to improve the number of system units that can be processed in parallel within a flow cell (e.g., up to 100 system units per flow cell, greater than 100 system units per flow cell, etc.); operations to mitigate bubble formation-associated effects within a flow cell; operations associated with optimization of ligation, denaturing, and washing steps (e.g., during in-situ sequencing); and/or other suitable quality control operations.

### 4. Methods and Example Applications of Use

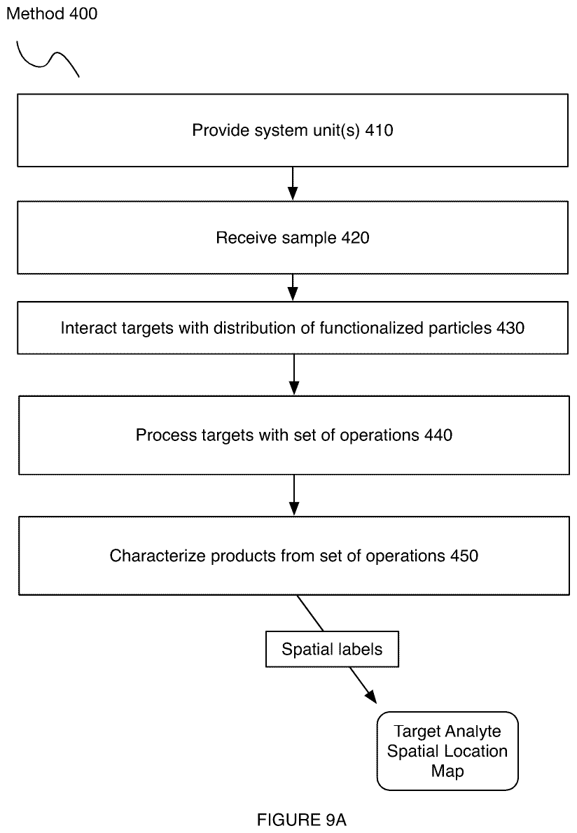

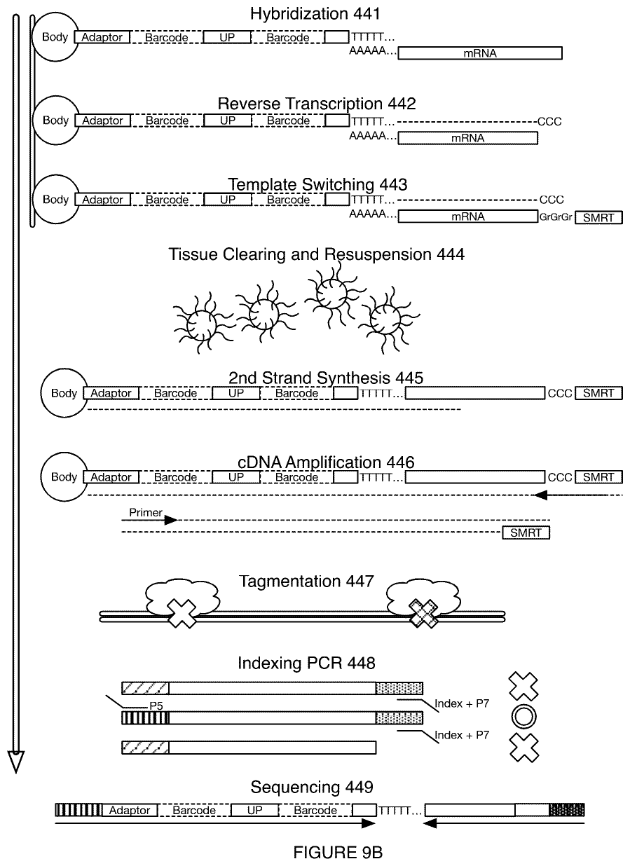

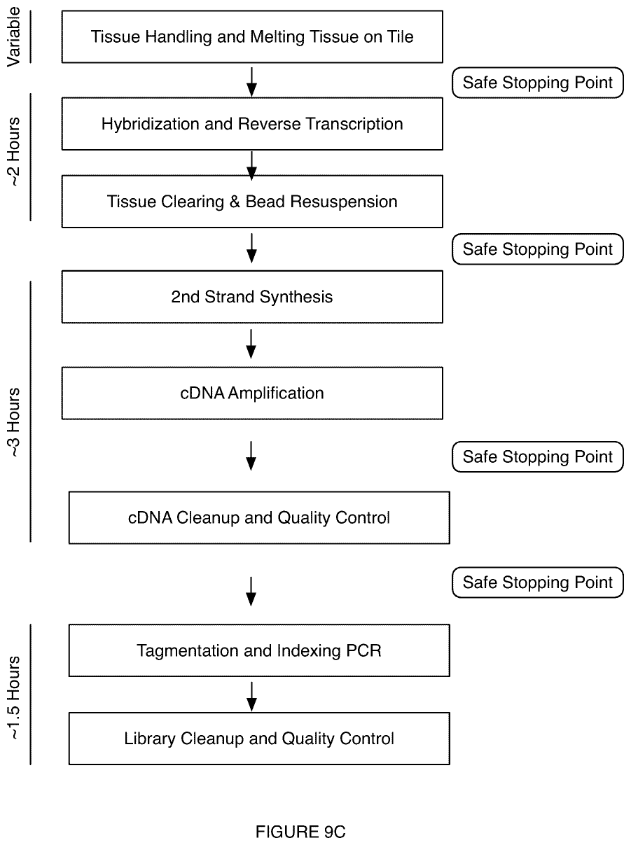

As shown in FIG. 9A, an embodiment of method 400 for characterizing locations of target analytes of a sample can include: providing a substrate having a distribution of functionalized particles coupled to the substrate, wherein each of the distribution of functionalized particles comprises a body, optionally a first cleavable linker coupling the body to the substrate, and one or more molecules coupled to the body, the one or more molecules comprising a capture segment, a barcode segment (e.g., stochastic barcode sequence described), and optionally a second cleavable linker coupled to the body 410; receiving the sample at the substrate 420; promotion interactions with target analytes of the sample by way of the distribution of functionalized particles at the substrate 430, upon promoting interactions between the distribution of targets of the sample and the distribution of functionalized particles; applying a set of reactions to the sample at the substrate, thereby processing target analytes of the sample with a set of operations 440; and characterizing products resulting from the set of operations 450 (e.g., obtaining a set of sequences of a population of molecules generated from the set of reactions, the set of sequences associated with the distribution of targets labeled using the stochastic barcode sequences of the distribution of functionalized particles) in order to return a set of positions of the distribution of targets.

The method 400 functions to process one or more samples in order to characterize spatial locations of target analytes of the sample. The method 400 can additionally or alternatively provide other suitable functions (e.g., in relation to diagnostics for various pathologies, in relation to characterization of target analytes of a tissue, in relation to characterization of target analytes of distributions of single cells, etc.).

The method 400 can generate high-resolution spatial maps of targets of the sample(s), where, in examples, the method 400 can achieve resolutions of: greater than one target mapped per 500 um², greater than one target mapped per 400 um², greater than one target mapped per 300 um², greater than one target mapped per 200 um², greater than one target mapped per 150 um², greater than one target mapped per 100 um², greater than one target mapped per 50 um², greater than one target mapped per 40 um², greater than one target mapped per 30 um², greater than one target mapped per 20 um², greater than one target mapped per 10 um², or any intermediate number of targets mapped per unit area.

Mapping can be performing for each of a set of at least 2 targets, 3 targets, 4 targets, 5 targets, 6 targets, 7 targets, 8 targets, 9 targets, 10 targets, 11 targets, 12 targets, 13 targets, 14 targets, 15 targets, 16 targets, 17 targets, 18 targets, 19 targets, 20 targets, 25 targets, 30 targets, 40 targets, 50 targets, 100 targets, 1000 targets or any intermediate number of targets simultaneously, at resolutions described.

Generated maps can have an associated signal-to-noise ratio (SNR) of at least 100, 200, 300, 400, 500, 600, 700, 800, 900, 1000, 2000, 3000, 4000, 5000, 6000, 7000, 8000, 9000, 10000, 100000, or greater, where background noise is attributed to leakage of targets away from their original positions at the sample and toward functionalized particles that are positioned further away from the original positions at the sample. As such, SNR can be determined by calculating a ratio between a number of signal copies observed at “correct” positions and a number of background copies of targets present in “background” positions). In variations, determining the SNR can include: identifying one or a set of genes known to be expressed in regions (e.g., single cell types, single cell subtypes) of a sample (where such genes can be expressed at low level, such as less than 100 copies per particle, or at high level); quantifying expression of the one gene or the set of genes across the sample (e.g., according to method steps described below), determining a value of the signal from a measure of the expression of the one gene or the set of genes in regions that should express such gene(s), and determining a value of the noise from a measure of the expression of the one gene or the set of genes in regions that should not express such genes. The SNR can then be determined from the value of the signal divided by the value of the noise. In a specific example, for a mouse hippocampus sample, the SNR was determined using hippocalcin hpca) gene and transthyretin (ttr) gene, which are known to be expressed by certain hippocampus cell subtypes and not represented in other sample regions. Using hpca and ttr, the value of the noise was determined to be 0, indicating that hpca and ttr genes were not observed in regions that should not express hpca/ttr. As such, the SNR for the exemplary sample type was shown to be infinite. Values of signal and noise can be determined from raw or normalized counts. Higher levels of noise can be attributed to anchoring of oligonucleotides onto glass substrates directly (e.g., without a particle layer intermediary), which can create different surface physical characteristics that promote target leakage and thus greater levels of background noise.

Generated maps can have an associated false positive rate less than a threshold number (e.g., a false positive percentage), where the false positive rate is determined from a percent (e.g., x %) of positive copies of targets observed beyond a threshold distance (e.g., y micrometers) away from where the positive copies (“signal”) should actually originate from (e.g., originating positions of targets in the sample). In examples, the false positive rate can be less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, or less than another suitable percent. In examples, the threshold distance can be 15 micrometers, 14 micrometers, 13 micrometers, 12 micrometers, 11 micrometers, 10 micrometers, 9 micrometers, 8 micrometers, 7 micrometers, 6 micrometers, 5 micrometers, 4 micrometers, 3 micrometers, 2 micrometers, or another suitable threshold distance, where background noise is attributed to leakage of targets away from their original positions at the sample and toward functionalized particles that are positioned further away from the original positions at the sample. As such, the method 400 can include generating a spatial map of a distribution of targets of a tissue sample, wherein upon generating the spatial map by a set of processes, wherein the set of processes comprises: receiving the tissue sample at a substrate comprising a distribution of functionalized particles arranged in a random close packed configuration, each of the distribution of functionalized particles comprising a stochastic barcode sequence paired with a position on the substrate, applying a set of reactions to the tissue sample at the substrate, obtaining a set of sequences of a population of molecules generated from the set of reactions, the set of sequences associated with the distribution of targets labeled using the stochastic barcode sequences of the distribution of functionalized particles, returning a set of positions of the distribution of targets upon processing the set of sequences, identifying a gene with a known expression profile across regions of the tissue sample, and determining the false positive rate upon quantifying expression of the gene across the sample based upon the set of positions of the distribution of targets.

The method 400 can further achieve generation of spatial maps that have a resolution of less than a threshold distance between features (e.g., beads or other particle bodies, rods, protrusions, recesses, ridges, valleys, channels, wells, oligonucleotide spots, etc.) of a substrate for target capture/interactions. Embodiments, variations, and examples of spatial maps generated have a resolution of less than 50 picometers between features, less than 40 picometers between features, less than 30 picometers between features, less than 20 picometers between features, less than 10 picometers between features, less than 5 picometers between features, or less than 1 picometer between features

In particular, in relation to platforms involving non-close-packed features (e.g., functionalized particles) for target capture and mapping, the invention(s) described achieve high resolution mapping with minimal (or non-existent noise, as described above), by having smaller functional unit areas and spatial unit areas for target capture. Furthermore, the systems described minimize the amount of empty or dead space between features for target capture, by close packing such features (e.g., in comparison to platforms where features are printed as spots that are spaced apart at the surface of a substrate, such as a glass slide). In examples, as shown in FIG. 1C, structural configurations of the features for target mapping, as in the inventions described, produce smaller capture areas per feature (and therefore higher resolution data), smaller spatial unit areas, smaller functional unit areas, less leakage of targets from the sample and therefore lower levels of background noise, and the ability to identify features (e.g., single cell subtypes) based upon a clustering analysis of spatial biomarkers, and without requiring deconvolution or other more involved computational approaches.

In embodiments, the target analytes characterized spatially according to the method 400 can include one or more of: nucleic acid material (e.g., DNA, RNA, miRNA, etc.), protein material, amino acid material, other small molecules, other single analytes, other multianalytes, and/or other suitable target material of a sample. In embodiments, the sample can include whole tissue structures, tissue portions (e.g., histological tissue slices, formalin-fixed paraffin-embedded (FFPE) tissue, frozen tissue, biopsied tissues, fresh frozen plasma, seeded natural scaffolds, seeded synthetic scaffolds, etc.), organs, whole organisms, organoids, cell suspensions (e.g., frozen cell suspensions that are separated prior to processing with the system, cell suspensions retained in a medium/hydrogel medium, etc.), nuclei suspension, single cells, organelles, sub-organelle structures, intra-organelle components, mitochondrial targets, viruses, microorganisms, and other samples.

Slices of tissues, cells, or suspensions (e.g., cell suspensions, nuclei suspensions, etc.) can be 2 micrometers thick, 3 micrometers thick, 4 micrometers thick, 5 micrometers thick, 6 micrometers thick, 7 micrometers thick, 8 micrometers thick, 9 micrometers thick, 10 micrometers thick, 11 micrometers thick, 12 micrometers thick, 13 micrometers thick, 14 micrometers thick, 15 micrometers thick, 16 micrometers thick, 17 micrometers thick, 18 micrometers thick, 19 micrometers thick, 20 micrometers thick, 25 micrometers thick, 30 micrometers thick, or of other suitable thickness.

Slices can be generated using a microtome system, free-hand sectioning, tissue slicers for submerged samples, tissue dissociation systems, and/or other techniques.

For cell or nuclei suspensions, chosen sample thickness can improve recovery rate of captured nuclei targets (e.g., percent of nuclei targets mapped in comparison to actual number of nuclei targets), based upon exposure of nuclei, ability to separate samples from functionalized particles at a substrate, or other factors. Additionally or alternatively, samples can be sandwiched between different substrates with functionalized particles, in order to improve recovery rate of captured nuclei targets using the distribution of functionalized particles. Recovery rate of nuclei targets can additionally or alternatively be improved by implementation of magnetic functionalized particles (e.g., which can be applied to samples with force, injected into samples, or otherwise used), where magnetic retrieval after target capture can result in improved recovery rates. Functionalized particles (e.g., magnetic functionalized particles) can implement antibodies configured against nuclear membrane components, to further enhance interactions with nuclei targets and improve recovery rates. Sample processing can further include use of electroporation (e.g., application of an electric field) or membrane permeabilization techniques when samples are in contact with functionalized particles, in order to increase access to nuclei for capture of nuclei targets for mapping (in addition to cytoplasmic targets). Viral vectors can also be used in order to deliver capture probes for nuclei targets.

In embodiments involving sandwiching of samples between two substrates, in order to capture targets at both sides of the sample, one or both substrates can include features that allow reagents to access interior sample portions during sample processing. In variations, one or both substrates can be composed of a porous material (e.g., porous glass) that allows reagents to cross the substrates) to interior sample portions. Additionally or alternatively, in variations, reagents can be stored within bodies of functionalized particles (e.g., as vesicles), where controlled release of such reagents in response to a trigger (e.g., chemical stimulus, mechanical stimulus, light stimulus, pH stimulus, temperature stimulus, etc.) allows reagents to access interior sample portions. Additionally or alternatively, bodies of particles (e.g., the functionalized particles, particles positioned among the functionalized particles and configured to be sacrificial) can be controllably degraded in response to a trigger (e.g., chemical stimulus, mechanical stimulus, light stimulus, pH stimulus, temperature stimulus, etc.), in order to allow reagent penetration through spaces created upon degradation, to interior sample portions.

Samples can be further processed to efficiently isolate nuclei of a sample (before and/or after spatial tagging of nuclei). In specific examples, nuclei isolation efficiency can be achieved using improved buffer compositions, using mechanical shearing of tissue to expose nuclei, using photocleaving parameters to improve nuclei tagging efficiency, and/or other nuclei isolation mechanisms. For instance, nuclei can be coupled to buoyant particles (e.g., microbubbles) functionalized to bind to/capture nuclei, which can be retrieved by buoyancy-based separation (e.g., nuclei bound to such buoyant particles float and are thus isolated from other sample components). Additionally or alternatively, nuclei can be coupled to magnetic particles (e.g., magnetic microparticles, magnetic nanoparticles, etc.) functionalized to bind to/capture nuclei, which can be retrieved by magnetic-based separation (e.g., nuclei bound to such magnetic particles are isolatable from other sample components upon application of a magnetic field).

Samples can further be processed in other suitable manners prior to interactions with the system. For instance, sample processing can include one or more of: preserving sample material (e.g., through freezing, through fixing, through embedding, etc.), lysing sampling material, washing sample material, inducing cell/tissue swelling/expansion or shrinking (e.g., through hypertonic/hypotonic solutions), inducing cell/tissue gelling, clarifying cells/tissues (e.g., using lipid clarification), and/or other suitable processing steps. In relation to mapping of nuclei, sample processing can include freezing of nuclei (e.g., in suspension, in a layer), followed by application onto a substrate with functionalized particles, and optionally, sealing of the nuclei sample at the substrate with a membrane or material (e.g., gel material). In order to improve recovery and/or loss of particles, samples can be gently fixed, in order to reduce stickiness and support ease of peeling a sample from a substrate with functionalized particles, for further processing. Further processing can include using a device to further expose nuclei in order to improve recovery rate of nuclei targets, where such a device can homogenize samples in a manner that preserves and enables separation of nuclei (e.g., with nuclei targets captured using functionalized particles described below) for further characterization and mapping.

In relation to frozen sample material, the method 400 can include freezing of sample material in a manner that lyses cell membranes and/or other sample structures. Alternatively, the method 400 can include freezing of sample material in a manner that preserves cell membranes and/or other sample structures. For instance, freezing in a preserving manner can implement one or more of: rapid freezing (e.g., in liquid nitrogen, in another freezing medium, at another freezing temperature); nucleating proteins, low molecular weight solutes, saccharides (e.g., glucose), or other compounds that draw water from cells (thereby reducing the amount of water turned to ice and reducing volumetric expansion during freezing); and/or other anti-freeze compounds, in order to implement the method without lysis or structural compromise (e.g., with respect to characterizing surface target analytes without disrupting original structures, etc.). Affecting the nature of sample freezing can further affect water volume and/or analyte concentration during sample processing.

In variations, captured target analytes can be processed and observed upon harvesting such target analytes and/or their derivatives after they have interacted with embodiments, variations, and examples of the system(s) and support structure(s) described above. Additionally or alternatively, the method can implement steps for observing and mapping locations of target analytes in space without harvesting of target analytes or derivatives from host tissues, cells, or other host material.

In some non-limiting examples, sample material from which targets can be captured and processed according to embodiments of the method 400 can include natural tissue including one or more of: nervous system biological material (e.g., brain tissue, spinal cord tissue, nerve tissue, etc.) spanning single or multiple layers (e.g., cortical layers) of tissue and/or in relation to different types of neurons (e.g., excitatory neurons, inhibitory neurons), lymphatic system biological material (e.g., spleen tissue, lymph material, tonsil tissue, etc.) spanning zone 1, zone 2, and/or zone 3 tissue, cardiovascular system biological material, integumentary system biological material, skeletal system biological material, muscular system biological material, respiratory system biological material, digestive system biological material, endocrine system biological material, urinary system biological material, and reproductive system biological material. Additionally or alternatively, sample material can include plant tissue material, fungal tissue material, or other material. Cellular material can be associated with normal and diseased states, including one or more of: cancer cells, circulating tumor cells, metastatic cells, benign cells, or any combination thereof. In embodiments, the sample can include solid/contiguous tissue material obtained from a subject.

In some non-limiting examples, sample material from which targets can be captured and processed according to the method 400 can include synthetic tissue including cell-seeded scaffolds or other composite material.

Receiving the sample at the substrate 420 can additionally or alternatively include implementing one or more structures for retention of the sample in position relative to the functionalized particles, where, in examples, structures can include substrates (e.g., substrates patterned with the distribution of functionalized particles), microwells, microarrays (e.g., with nucleic acids capturing particles), scaffolds, or other 2D/3D structures. Additionally or alternatively, one or more of the sample and the functionalized particles can be retained in position by use of forces (e.g., magnetic forces, electrical forces/charged surfaces, gravitational forces, forces applied using acoustic or other vibration, centrifugal forces, buoyancy forces, chemical binding, etc.). In such variations, retention can be reversed by releasing retained functionalized particles from a support structure or substrate by one or more of: application of magnetic forces of reverse polarity or removal of a magnetic field (e.g., for functionalized magnetic particles), application of reverse polarity charge or other removal of electrical forces, removal of gravitational forces, removal of forces applied using acoustic or other vibration, application of a detergent to remove chemical bonds, and/or other suitable mechanisms. As such, retention and release of a sample from a substrate can be performed in a reversible or non-reversible manner (e.g., to facilitate enzymatic reactions at the substrate and/or within a process container after transfer of the substrate to the process container).

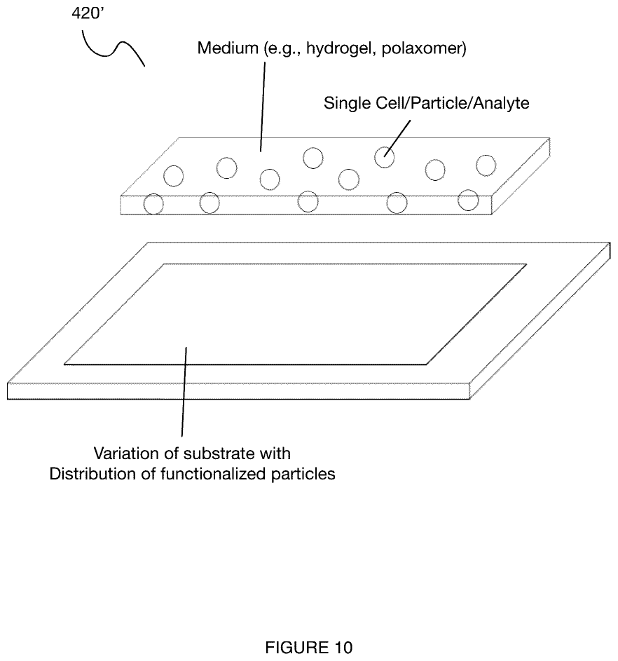

As shown in FIG. 10, a variation of receiving the sample at the substrate 420′ can include receiving a composite sample, including a distribution of single cells (or alternatively, single particles, single, analytes, etc.) distributed within or across a medium (e.g., hydrogel medium, polaxomer medium), at the substrate, and performing embodiments, variations, and examples, of the method(s) described accordingly. Such a variation can thus enable single-cell or single particle spatial multi-omics without droplet-based or microwell-based systems, thereby producing shorter hands on time and/or less complex single-particle processing apparatus. Furthermore, such a variation can implement various substrate sizes in order to overcome doublet, triplet, quadruplet, etc. rates and increase throughput.

Additionally or alternatively, promoting interactions between functionalized particles of the system and a sample can include infusing functionalized particles and/or a system unit into or onto a sample (e.g., into a tissue, into an organ, etc.). Examples of infusion can include one or more of: injection, electroporation, use of vectors (e.g., viral vectors), and other infusion methods. Additionally or alternatively, Additionally or alternatively, promoting interactions between functionalized particles of the system and a sample can include coupling functionalized particles to a surface of a sample/specimen (e.g., by chemical binding, by magnetic binding, by other binding).

Additionally or alternatively, during use, and in an application of use involving spatial characterization of target analytes in 3D, stacks of substrates with distributions of functionalized particles can be implemented (e.g., with layering of samples/slices of tissue and units of the system 100, with disassembly of sample into sub-portions and interacting sub-portions with various substrates). As such, the system 100 can include additional substrates with distributions of functionalized particles (e.g., a second substrate with a second distribution of functionalized particles, a third substrate with a third distribution of functionalized particles, etc.), with layering or re-assembly of sample pieces and reconstruction of 3D volumes by stitching data derived from implementation of the various substrates.

Additionally or alternatively, during use, and in an application of use involving spatial characterization of target analytes in 3D, methods described can include applying units of the system 100 to a set of sides of a sample (e.g., block of tissue), followed by promoting interactions between the sample and functionalized particles of each unit of the system 100, and reconstructing 2D surfaces and/or 3D volumes by stitching data derived from implementation of the substrates of the units of the system 100. In variations, application of units of the system 100 to a set of sides of the sample can include providing a support structure 200 configured to fold about the set of sides of the sample (e.g., with an origami structure that folds to apply units of the substrate 110 to the set of sides of the sample, and unfolds to release the sample, etc.). Additionally or alternatively, the support structure 200 can be constructed with a shape memory material that responds to environmental conditions (e.g., temperature, electric field, pH, etc.) and adjusts morphology to contact the set of sides of the sample, and/or responds to environmental conditions (e.g., temperature, electric field, pH, etc.) and adjusts morphology to displace units of the system 100 from the set of sides of the sample (e.g., to release the sample for further processing). Such elements and configurations can thus be used to generate spatial distributions of targets of a sample, for samples that have a low level of rigidity. In examples, such tissues can have a Young's Modulus less than 50 kPa, less than 40 kPa, less than 30 kPa, 20 kPa, less than 15 kPa, less than 10 kPa, less than 9 kPa, less than 8 kPa, less than 7 kPa, less than 6 kPa, less than 5 kPa, less than 4 kPa, less than 3 kPa, less than 2 kPa, less than 1 kPa, or other suitable values. Additionally or alternatively, such elements and configurations can be used to generate spatial distributions of targets of a sample, for samples that have a high level of rigidity. In examples, such tissues can have a Young's Modulus greater than 50 kPa, greater than 100 kPa, greater than 1 MPa, greater than 50 MPa, greater than 100 MPa, greater than 500 MPa, greater than 1 GPa, greater than 10 GPa, greater than 20 GPa, greater than 30 GPa, greater than 40 GPa, greater than 50 GPa, greater than 60 GPa, greater than 70 GPa, greater than 80 GPa, greater than 90 GPa, greater than 100 GPa, or other suitable values.

Additionally or alternatively, the method can implement mapping molecules (e.g., delivered using a viral library encoding a diverse collection of RNA sequences) that interact with corresponding sample targets of the sample (e.g., through interactions with exposed target projection regions of the sample), and tracking the mapping molecules (e.g., through downstream sequencing processes) upon promoting an interaction between the sample and a unit of the system described above.

Additionally or alternatively, the method can implement structures (e.g., mesh structures with affinity molecules, mesh structures with primer-like sequences, etc.) positioned in proximity to a sample, where target analytes transfer to the structures during sample processing, and are subsequently processed using a unit of the system described above (e.g., by promoting interactions between the mesh structure and the unit of the system).

Steps 410 through 430 can implement embodiments of systems and support structures described above, and/or other suitable system elements, some embodiments, variations, and examples of which are described in U.S. application Ser. No. 17/376,396 incorporated by reference above.

In some embodiments, as shown in FIG. 9B, applying a set of reactions to the sample at the substrate, can include performing a hybridization operation 441 between target material of the sample and the capture segments of the functionalized particles (e.g., at the substrate); performing a reverse transcription operation 442 upon outputs of the hybridization operation (e.g., at the substrate); performing a template switching operation 443 upon outputs of the reverse transcription operation (e.g., at the substrate); performing a tissue clearing operation with particle resuspension 444 (e.g., upon displacing the substrate after step 443 into the process container 250); performing a second strand synthesis operation 445 (e.g., within a process container 250); performing cDNA amplification operation 446 with outputs of the second strand synthesis operation 445 (e.g., within a process container 250); performing a tagmentation operation 447 with outputs of the cDNA amplification operation (e.g., within a process container 250); performing an indexing PCR operation 448 with outputs of the tagmentation operation (e.g., within a process container 250); and performing a sequencing operation 449 with outputs of the indexing PCR operation.

Variations of the set of reactions performed in step 440 can include one or more of: performing the second strand synthesis operation at the substrate (e.g., prior to or with omission of tissue clearing and particle resuspension); implementation of betaine, PEG or other molecular crowding agents, formamide, DMSO, and magnesium chloride for hybridization and reverse transcription operations; freezing the sample at the substrate post-application of the sample to the functionalized particles at the substrate, where freezing can be performed at a desired temperature (e.g., at 0° C., at −20° C., at −80° C., etc.) for a duration of time (e.g., 4 days, less than 4 days, more than 4 days) prior to performing subsequent processing steps (e.g., post-thaw) for target mapping, without significant degradation in particle robustness and mapping performance (e.g., in relation to performance and quality metrics described); implementation of terminal transferase enzyme (e.g., in TAS-seq) to generate a second strand (as an alternative to second strand synthesis as described); implementation for RNA fragmentation and A-tailing to capture more RNA material and/or desired regions of RNA material (e.g., non-coding, non-3′ ends of mRNA material) of the sample (e.g., in VASA-seq); implementing immune repertoire VDJ assays (e.g., generating a VDJ recognition site) with template switching and long-read sequencing capabilities; performing probe-ligation for a targeted DNA panel assay, with permeabilization to access DNAs (e.g., using methanol fixation with permeabilization instead of other fixatives, with saponin, Triton X-100, NP40, Tween, etc.).

Variations of the reactions applied in step 440 can additionally or alternatively include executing an antibody-protein sequencing (Ab-seq) workflow with oligonucleotide-conjugated antibodies to examine protein expression. In one variation, the Ab-seq workflow can include performing a fixation operation (e.g., with formalin, with an alcohol such as methanol, etc.) and Ab-seq staining for the sample (e.g., a free-floating sample post-sectioning, a sample applied to a glass slide, a sample applied to a semi-permeable membrane or filter, etc.), followed by a hybridization operation with capture segments of functionalized particles (e.g., with maintenance of contact between the sample and the functionalized particles at the substrate). The Ab-seq workflow can include operations including: a polyA-based capture operation (or alternatively, a ligation-based capture operation), followed by a reverse transcription operation, a template switching operation, a tissue clearing operation, a second strand synthesis operation, a cDNA amplification operation (e.g., with Truseq/SMRT for mRNAs and Truseq/Abseq for AbOligonucleotides), separation of mRNA products from Ab-seq products by double sided cleanup, multiple rounds of PCR for AbOligonucleotide targets, and a tagmentation operation followed by indexing PCR for mRNA targets. Final PCR steps can be followed by next generation sequencing to combine RNA-seq and Ab-seq data outputs to characterize the sample.

Variations of the Ab-seq workflow can include one or more of: determining regions of interest (ROI) for examination by using registration features and/or fiducials to align a sample with the distribution of functionalized particles at the substrate (e.g., lining up sample features with registration features of the system 100 at the corners or other locations of the distribution of functionalized particles; dT blocking and de-blocking operations (e.g., at 55° C., with a washing step); performing Ab-seq with fluorophore staining (e.g., with Cy5 staining) as a quality control method for evaluating staining conditions and antibody titration); performing Ab-seq for adjacent sections, and performing stitching operations upon retrieved data in order to generate 3D characterizations of relevant biomarkers; performing Ab-seq with different stains for the same sample section; and performing other suitable operations.

In some embodiments, processing target analytes and characterization in steps 440 and 450 can include read-out/detection and identification of spatial labels/stochastic barcodes associated with target analytes by one or more of: sequencing (e.g., sequencing with error-reduction by dynamic annealing and ligation (SEDAL); sequencing by hybridization; sequencing by ligation; sequencing by polymerization; hybridization of fluorescent probes against barcode sequences (e.g., using Nanostring technology); PCR performing read-out operations of barcoded analytes (e.g., for applications with mRNA capture and next generation sequencing (NGS) read-out, for applications with reverse transcription followed by cDNA amplification followed by generation of NGS libraries, etc.); implementing documentation associating barcode sequences with locations of functionalized particles (e.g., by reading functionalized particle barcode sequences and transcript identities by NGS or other sequencing, then associate the barcode/transcript identities with the spatial location from the spatial decoding process performed during manufacturing); and performing other suitable steps. Identification of spatial labels/barcodes, in combination of detection of signals derived from target analytes captured at the distribution of functionalized particles can thus enable characterization of locations of the target analytes in space.

In variations, the method 400 can further include returning outputs based upon processing and characterization in steps 440 and 440. In example applications, returning outputs can include one or more of: returning an output characterizing a stage of cancer (e.g., upon identifying a set of cancer genes and/or spatial distributions thereof) associated with the sample; returning an output characterizing a somatic mutation associated with the sample; returning an output characterizing an immune response associated with the sample; returning an output characterizing a stage of biological development (e.g., development stage associated with clustering of mRNAs, etc.) associated with the sample; returning an output characterizing a pathological state (e.g., associated with liver disease, associated with kidney disease, associated with a neurological disease, associated with another disease state, performing diagnostics without whole transcriptome assessment etc.) associated with the sample; returning an output characterizing a spatial characteristics of a whole transcriptome associated with the sample; returning outputs characterizing gene expression of a targeted set of genes; returning outputs characterizing protein expression (e.g., via oligonucleotide-conjugated antibodies), returning outputs characterizing nucleosomal positioning (e.g., with ATAC-seq), returning outputs characterizing methylation sequences, returning outputs characterizing chromatin structure (e.g., with characterization of open chromatin, with characterization of chromatin accessibility, etc.), returning outputs characterizing transcription factor binding, returning outputs characterizing genomic features (e.g., mutations, copy number variations, etc.), and/or returning other suitable outputs.

In examples, the systems and methods described provide transcriptome-wide RNA (i.e., whole transcriptome) sequencing information with single-cell spatial resolution or better, with a rapid workflow (e.g., processing time from tissue sectioning, to reverse transcription, to library amplification within 3 hours, within 2.5 hours, or less).

Additionally or alternatively, returned outputs and/or processing steps implemented according to the method 400 can be used to associate genotypic information with phenotypic information. In one example use case, (e.g., adapted from neuroscience applications), a phenotype of a target region can be controlled by optogenetics and/or observed in another suitable manner (e.g., using electrophysiology, using magnetic encephalography, etc.). The target region can then be analyzed with one or more units of the system to acquire spatial genomic information and to generate associations between phenotypic information and genomic information. Such methods can further be applied to other sample types, with or without stimulation of the sample.

As described, spatial characterization can be performed in 2D and/or 3D (e.g., with 3D structures and/or layering of system units with sample slices, etc.). Furthermore, a set of samples can be processed for a set of subjects/patients in parallel, using different subject/patient barcodes (e.g., molecular barcodes) in a manner that allows for decoding of characterizations corresponding to respective subjects/patients in an efficient manner.

Variations of the method 400 can include steps for mitigation of issues associated with movement of targets away from nearby functionalized particles, and subsequent capture of targets at distant functionalized particles during sample processing steps. Target movement and capture can be attributed to one or more of: presence of highly-expressed targets (e.g., target genes); diffusion or directional flow of targets or functionalized molecules away from originating positions, due to sample processing steps and/or apparatuses that allow for target flow away from originating positions within a sample; and provision of a distribution of functionalized particles having a substantially larger footprint (e.g., attributed area, number of functionalized particles, etc.) than a footprint occupied by the sample.

Mitigation of smearing can be enabled by one or more of: increasing density or number of available capture sites on functionalized molecules used to spatially tag targets of the sample; increasing volumes of chambers used for hybridization and washing steps, in order to reduce undesired hybridization of target molecules (e.g., of highly-expressed genes) to non-nearby functionalized particles; matching an area (or attempting to match an area) of a footprint of a distribution of functionalized particles with a footprint of a sample; blocking capture molecules of exposed functionalized particles of a distribution of functionalized particles after applying a sample to the distribution of functionalized particles, where exposed functionalized particles include particles not covered by the sample, and where blocking capture molecules comprises adding adenosine (dA) blockers (or other blockers) to hybridization buffers used during assay workflows; implementing agitation of solutions involved in hybridization, during hybridization steps of assay workflows; applying less efficient hybridization buffers and/or longer incubation times, in order to prevent leakage; and performing other suitable operations.

In examples, directional flow of targets, which can result in a smearing effect in generated maps, was correlated with orientation of the substrate within a chamber containing hybridization buffer for hybridization steps, where a predominant direction of transmission of a system unit into a chamber of hybridization buffer, and/or a predominant direction of removal of a system unit from a chamber of hybridization buffer is correlated with the direction of smearing (e.g., loose/unbound targets are moved and recaptured during transmission or removal from hybridization buffer).

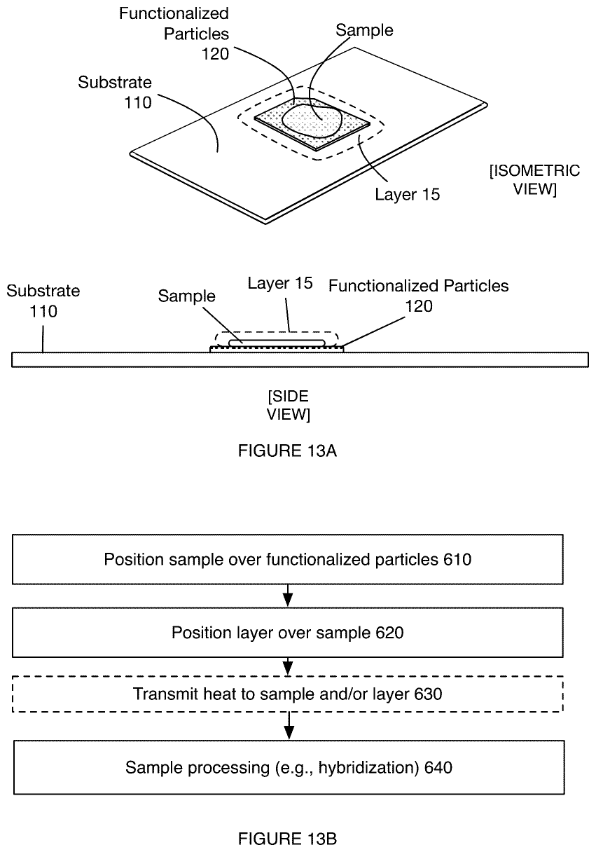

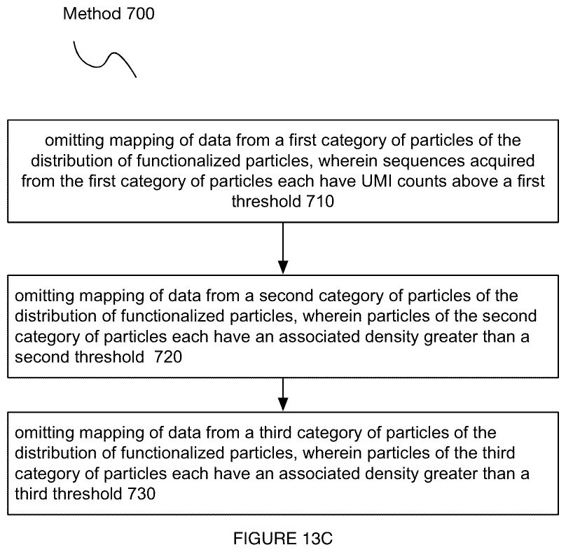

In one variation, methods involving hybridization of targets to functionalized particles can include application of a smear-prevention layer 15 (e.g., smear-prevention layer) over the sample, such that the sample is sandwiched between the smear-prevention layer 15 and the distribution of functionalized particles (e.g., during hybridization steps) to prevent target drift and/or to prevent smearing in maps generated from processes and reactions involving the distributions(s) of functionalized particles (see FIG. 13A).

In one variation, the smear-prevention layer 15 can include a layer of optimal cutting temperature (OCT) compound, and methods for application of the smear-prevention layer 15 (e.g., of OCT compound) can include: positioning a sample (e.g., frozen tissue slice, other sample) at the substrate over at least a portion of the distribution of functionalized particles 610; positioning a layer (e.g., a layer of OCT compound) over the sample 620; optionally transmitting heat to the sample and to the layer 630; and performing subsequent sample processing steps (e.g., hybridization steps) 640, as described (see FIG. 13B). In variations, methods for application of the layer can include re-freezing the sample and the layer (e.g., of OCT compound) prior to subsequent processing.

In variations, the layer (e.g., of OCT compound) can be generated from a solidified (e.g., frozen) body of material, where the layer can be sectioned from the solidified body. In variations, sectioning can be performed using one or more of: cutting, laser cutting, shaving, or another suitable method. In variations, the smear-prevention layer 15 may not be a section, and can alternatively be applied by aspiration and delivery, spraying, coating, spinning, or other methods of application of a liquid volume of material of the layer.

In variations, the thickness of the layer can be: 2 micrometers thick, 3 micrometers thick, 4 micrometers thick, 5 micrometers thick, 6 micrometers thick, 7 micrometers thick, 8 micrometers thick, 9 micrometers thick, 10 micrometers thick, 11 micrometers thick, 12 micrometers thick, 13 micrometers thick, 14 micrometers thick, 14 micrometers thick, 15 micrometers thick, 16 micrometers thick, 17 micrometers thick, 18 micrometers thick, 19 micrometers thick, 20 micrometers thick, 21 micrometers thick, 22 micrometers thick, 23 micrometers thick, 24 micrometers thick, 25 micrometers thick, 26 micrometers thick, 26 micrometers thick, 27 micrometers thick, 28 micrometers thick, 29 micrometers thick, 30 micrometers thick, 35 micrometers thick, 40 micrometers thick, 50 micrometers thick, 60 micrometers thick, a thickness intermediate to thickness values described, or a thickness greater than 60 micrometers.

In variations, material of the layer (e.g., the OCT compound) can be diluted, which can improve diffusion of process reagents across the layer during sample processing while still preventing target drift and recapture. As such, the thickness of the layer can be adjusted and/or material of the layer can be diluted to provide suitable conditions for simultaneously achieving reagent transmission and target drift prevention. Dilution of the layer can be performed in a way that does not significantly affect temperature-associated performance aspects (e.g., freezing behavior, melting behavior, etc.) in relation to sample processing steps described.

In variations where application of the layer results in reduced sensitivity with respect to number of targets captured (e.g., targets captured at “correct positions”, without smearing) from the tissue sample, material of the layer can be combined with sample processing reagents associated with various capture steps and/or steps for promoting interactions between targets and functionalized particles.

In one example, material of the layer can include a combination of OCT compound with: a hybridization buffer, a sample lysis buffer, reagents for crosslinking, reagents for cleaving (e.g., of molecules with cleavage sites), and/or other suitable materials). As such, combination of the material(s) of the layer with process reagents can promote efficiency of sample processing steps, improve sensitivity of capture, and/or provide other suitable benefits.

Application of the layer (e.g., layer of OCT compound) can result in a percent reduction of smearing and background artifacts in generated maps, where in examples, the percent reduction in smearing was: greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, or greater. In examples, the percent reduction in smearing was determined by comparing values (e.g., percentage values, number values) of functionalized particles to which noise (e.g., noise associated with target movement and re-capture) was attributed, between a first section of a tissue sample and a second section of the tissue sample, where the smear-prevention layer was added to the first section, and was not added to the second section. In examples, the percent reduction in smearing was determined by comparing values (e.g., percentage values, number values) of UMIs/bead detected during sequencing, to which noise (e.g., noise associated with target movement and re-capture) was attributed, between a first section of a tissue sample and a second section of the tissue sample, where the smear-prevention layer was added to the first section, and was not added to the second section.

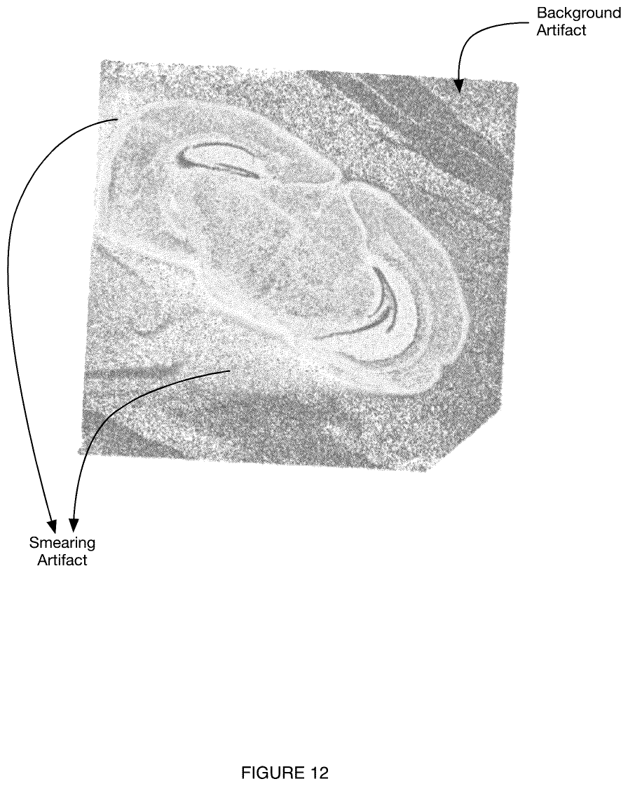

Mitigation of smearing can additionally or alternatively be enabled using bioinformatics-informed approaches to processing of data generated using units of the systems described, where data processing architecture can be structured to remove background signal artifacts and directional signal artifacts (which are observed as “smearing” in generated spatial maps), where background artifacts and/or directional signal artifacts can be attributed to leakage of targets away from originating positions, and subsequent recapture by capture molecules of the distribution of functionalized particles, as described above. An example of each type of artifact is shown in FIG. 12.

A method 700 for removing signal artifacts can include (after obtaining a set of sequences of a population of molecules generated from reactions involving molecules of the substrate): for a distribution of functionalized particles (or other features), omitting mapping of data from a first category of particles (or other features) of the distribution of functionalized particles (or other features), wherein sequences acquired from the first category of particles (or other features) each have UMI counts above a first threshold 710; omitting mapping of data from a second category of particles (or other features) of the distribution of functionalized particles (or other features), wherein particles (or other features) of the second category of particles (or other features) each have an associated density greater than a second threshold 720; and omitting mapping of data from a third category of particles (or other features) of the distribution of functionalized particles (or other features), wherein particles (or other features) of the third category (or other features) of particles each have an associated density greater than a third threshold 730 (FIG. 13C).

In variations, the first threshold can be a threshold of greater than 70 UMI counts, a threshold of greater than 75 UMI counts, a threshold of greater than 80 UMI counts, a threshold of greater than 85 UMI counts, a threshold of greater than 90 UMI counts, a threshold of greater than 95 UMI counts, a threshold of greater than 100 UMI counts, a threshold of greater than 105 UMI counts, a threshold of greater than 110 UMI counts, a threshold of greater than 120 UMI counts, a threshold of greater than 130 UMI counts, a threshold of greater than 140 UMI counts, a threshold of greater than 150 UMI counts, or another UMI count threshold.

In variations, the second threshold can be a threshold of greater than a first number of particles within a first area, wherein the first number of particles is 1 particle, 2 particles, 3 particles, 4 particles, 5 particles, 6 particles, 7 particles or greater; and wherein the first area is an area of 15 micrometers by 15 micrometers, 20 micrometers by 20 micrometers, 25 micrometers by 25 micrometers, 30 micrometers by 30 micrometers, 35 micrometers by 35 micrometers, 40 micrometers by 40 micrometers, 45 micrometers by 45 micrometers, 50 micrometers by 50 micrometers, 60 micrometers by 60 micrometers, 70 micrometers by 70 micrometers, or another suitable area. The second density threshold can be a finer density threshold, in comparison to the third threshold.

In variations, the third threshold can be a threshold of greater than a second number of particles within a second area, wherein the second number of particles is 7 particles, 8 particles, 9 particles, 10 particles, 11 particles, 12 particles, 13 particles, 14 particles, 15 particles, or greater; and wherein the second area is an area of 70 micrometers by 70 micrometers, 75 micrometers by 75 micrometers, 80 micrometers by 80 micrometers, 85 micrometers by 85 micrometers, 90 micrometers by 90 micrometers, 95 micrometers by 95 micrometers, 100 micrometers by 100 micrometers, 110 micrometers by 110 micrometers, 120 micrometers by 120 micrometers, 130 micrometers by 130 micrometers, 150 micrometers by 150 micrometers, 200 micrometers by 200 micrometers, or another suitable area. The second density threshold can be a coarser density threshold, in comparison to the second threshold.

In a specific example, the first threshold is a threshold of greater than or equal to 100 UMIs, the second threshold is a threshold of greater than 5 particles within a 40 micrometer by 40 micrometer area, and the third threshold is a threshold of greater than 10 particles within a 100 micrometer by 100 micrometer area.

Variations of the method 700 can involve adjustment of thresholds based upon sample type (e.g., tissue type), presence or absence of highly-expressed genes, substrate and functionalized particle characteristics (e.g., size of distribution of functionalized particles, footprint occupied by functionalized particles at the substrate, size of substrate relative to sample size, particle size(s), etc.), processing aspects (e.g., volumes and types of solutions used for hybridization, involvement of agitation during hybridization steps, sequencing depth, and/or other factors.

Variations of the method 700 can involve omission of data used in mapping, based upon other suitable thresholds, where thresholds can be based upon other parameters that distinguish desired signals for mapping, from background and directional signals. Additionally or alternatively, removal of artifacts can be performed with manual removal (e.g., with manual selection of data to be removed, through a user interface for transforming data into target maps) and/or clustering algorithms.

Variations of the method 700 can involve omitting/subtracting data from functionalized particles associated with a smear (for mapping purposes), and/or removing an in-tissue smear by subtracting characteristic transcriptomic patterns in the smear from the tissue.

The described bioinformatics approaches can result in a percent reduction of smearing and background artifacts in generated maps, where in examples, the percent reduction in smearing was: greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 95%, or 100%. In examples, the percent reduction in smearing was determined by comparing values (e.g., percentage values, number values) of functionalized particles to which noise (e.g., noise associated with target movement and re-capture) was attributed, between a first spatial map of targets generated without application of described bioinformatics processes, and a second spatial map of targets generated with application of described bioinformatics processes. In examples, the percent reduction in smearing was determined by comparing values (e.g., percentage values, number values) of UMIs/bead detected during sequencing, to which noise (e.g., noise associated with target movement and re-capture) was attributed, between a first spatial map of targets generated without application of described bioinformatics processes, and a second spatial map of targets generated with application of described bioinformatics processes.

The method 400 can include other suitable steps and/or enable other downstream applications.

### 5. Exemplary Workflows

Example protocols associated with the methods described involve generating high-quality, sequencing-ready libraries from samples (e.g., fresh frozen tissue samples, other sample types described), to generate high-resolution spatial transcriptomic maps for a sample. Specific exemplary workflows are described below.

### 5.1 Exemplary Workflow 1

An example protocol associated with the method 100 involves generating high-quality, sequencing-ready libraries from samples (e.g., fresh frozen tissue samples), to obtain high-resolution spatial transcriptomic information of a sample. Once tissues are sectioned and placed onto a substrate unit, the rest of the workflow can be completed in under eight hours in the example, with multiple safe stopping points. The workflow starts with hybridization of RNA to molecules of the functionalized particles on the substrate, followed by reverse transcription. A tissue-clearing step is performed to digest the tissue and release the beads from the substrate and into solution. Next, second strand synthesis is performed, followed by cDNA amplification. Finally, library preparation with a tagmentation process is used to generate sequencing platform-compatible libraries (e.g., Illumina™ platform compatible libraries). A schematic of the exemplary workflow, showing durations of time and safe stopping points is shown in FIG. 9C.

The exemplary method can be performed with a fresh frozen tissue sample, with prior assessment of RNA quality of the sample to extract an RNA integrity number (RIN). In examples the RIN can be compared against a threshold value (e.g., an RIN≥6, an RIN≥7, an RIN≥8, etc.) to determine if a sample should be used. In examples, tissue quality can be assessed by performing H&E staining on an adjacent tissue section to provide information on tissue structural context and sectioning quality. Details of exemplary method steps are further provided as follows:

### 5.1 Exemplary Workflow 1

Details of a first exemplary workflow are as follows:

### 5.1.1 Reagent and Apparatus Preparation

1. Thaw the following reagents at room temperature and keep them on ice until ready to use: RT/SS buffer (Reverse Transcription buffer; if precipitate is observed, heat the RT/SS buffer at 37° C. for five minutes and briefly vortex before use); dNTP (Keep RNase Inhibitor and RT Enzyme on ice).
2. Set one heat block to 52° C. and another to 37° C.
   5.1.2 Tissue Sectioning and Hybridization with Functionalized Particles of a Substrate
1. Equilibrate the fresh frozen tissue and OCT block (e.g., as an example of a smear-prevention layer 15) to −18° C. in a cryostat apparatus for at least 20 minutes prior to sectioning. The desired temperature for sectioning may vary depending on the tissue type.
2. Prepare the hybridization reaction mix following the table below and keep at room temperature:

| Component | 1 Reaction (μl) | Plus 5% overage (μl) |
|---|---|---|
| Hybridization buffer | 190 | 199.5 |
| RNAse inhibitor | 10 | 10.5 |
| Total | 200 | 210 |

3. Mount the tissue block and the OCT block onto cutting blocks with OCT compound.
4. Place the substrate unit in the cryostat to chill for at least one minute.
5. Record the Identifier of the substrate unit.
6. Section a 10 μm section of tissue.
7. Melt the section onto the substrate unit in one of the two ways described below:
7a. OPTION 1: For precise placement of the region of interest. Place the substrate unit in the cryostat to chill for 1 minute. Place the chilled substrate unit on the cutting stage and arrange the tissue section on top of the tile using a brush. Make sure that the region of interest is positioned directly over the substrate unit. With the substrate unit and tissue section facing up, melt the tissue section onto the substrate unit by moving the substrate unit off the cryostat stage, and GENTLY placing a finger on the bottom of the glass slide of the substrate unit. To avoid curling of the tissue, start from one side and slowly move your finger across the region rather than warming it from the center. A small brush can be used to hold the other end of the tissue flat during the initial melting from one end.
7b. OPTION 2: For quick placement of the region of interest. Hold a room temperature substrate unit in a holder with the substrate unit facing down. Hover the substrate unit over the region of interest. Keeping the substrate unit horizontal, gently lower the tile to bring it into contact with the tissue section. The tissue section should melt onto the tile immediately.
8. Place the substrate unit with the melted tissue section back into the cryostat.
9. Remove the tissue block and replace it with the OCT block.
10. Section a 30 μm section of the OCT block.
11. Move the OCT block section onto the tile so that it is covering the entire tile.
12. Melt the OCT block section onto the tile by placing a finger under the tile and moving it across the tile until the entire OCT block section and tissue section are melted. Alternatively, briefly warm the tile with the tissue section by placing a finger underneath the tile for a few seconds, and melt the OCT block section onto the tile with the stamping method described in step 7b.

SAFE STOPPING POINT. Substrate units can be stored at −80° C. for up to four days in a sealed container. To thaw the substrate unit, remove it from −80° C., warm up the substrate unit to room temperature by placing a finger under the substrate unit and ensure the tissue section has re-melted onto the substrate unit before proceeding to step 13.
13. Carefully remove the substrate unit from the blue adhesive with tweezers and place it in a 1.5 mL Eppendorf LoBind™ tube containing 200 μl of hybridization reaction mix. Make sure the substrate unit is completely submerged.
14. Incubate for 15 minutes at room temperature.
15. Remove the remaining block of tissue and OCT block from the cryostat and store them at −80° C.

### 5.1.3 Reverse Transcription

1. Prepare the 1×RT wash buffer following the table below for washing the substrate unit prior to RT and keep at room temperature:

| Component | 1 reaction (μl) | Plus 5% overage (μl) |
|---|---|---|
| RT/SS buffer | 40 | 42 |
| Nuclease-free water | 160 | 168 |
| Total | 200 | 210 |

2. Prepare the RT reaction mix following the table below and keep on ice:

| Component | 1 Reaction (μl) | Plus 5% overage (μl) |
|---|---|---|
| RT/SS buffer | 40 | 42 |
| dNTP | 20 | 21 |
| RNase inhibitor | 5 | 5.25 |
| RT enzyme | 10 | 10.5 |
| Nuclease-free water | 125 | 131.25 |
| Total | 200 | 210 |

3. Using a pair of clean tweezers, remove the substrate unit from the hybridization reaction mix and dip it in 1×RT wash buffer for three seconds.
4. Transfer the substrate unit to a new 1.5 mL tube containing 200 μl of RT reaction mix. Make sure the substrate unit is completely submerged.
5. Incubate the tube at room temperature for 10 minutes.
6. Move the tube to a heat block set at 52° C. and incubate for 30 minutes.

### 5.1.4 Tissue Clearing and Particle Resuspension

1. If precipitate is observed, heat the TC buffer at 37° C. for five minutes and briefly vortex before use.
2. Make the tissue clearing reaction mix following the table below and keep at room temperature:

| Component | 1 reaction (μl) | Plus 5% overage (μl) |
|---|---|---|
| TC buffer | 196 | 205.8 |
| TC enzyme | 4 | 4.2 |
| total | 200 | 210 |

3. Add 200 μl of tissue clearing reaction mix to the tube containing the substrate unit and RT reaction mix. Carefully pipette-mix 10 times without disrupting the tile.
4. Incubate at 37° C. for 30 minutes. If the tissue is difficult to digest or contains plant cell walls, you may wish to increase tissue clearing incubation from 30 minutes to a total duration of one hour.
5. Following the incubation, add 200 μl of bead wash buffer, pipette-mix repeatedly to dissociate beads from the substrate unit, and mechanically shear the tissue. Aim the pipette tip at the center of the bead patch when dissociating the beads from the glass slide. Occasionally, the residual tissue and beads may still be loosely associated after detachment from the substrate unit. Pipet up and down until the cloud of tissue and beads are fully dissociated. If the tissue persists, it may be placed back at 37° C. for an additional 10 minutes. There should be no visible patches of beads remaining on the glass. It is important to completely dissociate the beads from the substrate unit. Areas of the substrate unit that are not covered by tissue may be harder to dissociate. For areas that are harder to dissociate, mechanically dislodge the beads from the tile by gently brushing the tip sideways while the tip is parallel to the tile and pipetting across the tile where beads remain. Incomplete dissociation of beads not covered by tissue will not affect data quality and performance.
6. Guide the remaining piece of glass from the substrate unit to the top rim of the 1.5 mL tube with a pipet tip. Remove it from the tube with tweezers or forceps and discard.
7. Transfer the contents to a new 1.5 mL tube to ensure effective pelleting of the beads.
8. Spin the beads down for two mins at 3000×g. A white bead pellet should be visible to the eye.
9. Remove any bubbles from the top of the supernatant.
10. Carefully remove the supernatant without disturbing the bead pellet as shown above. Remove the supernatant immediately after centrifugation as the pellet may slide to the bottom of the tube after some time and become harder to visualize. When removing the supernatant, angle the tip away from the pellet and aspirate slowly to not disturb the pellet. Take care to prevent bead loss while removing supernatant. If the pellet begins to slide, spin for an additional 30 seconds at 3000×g. In order to retain as many beads a possible, it is not necessary to remove all of the supernatant from initial washes. 15-20 uL can be left. However, <10 μL of bead wash buffer should remain before adding a reaction mix to the beads.
11. Resuspend the bead pellet in 200 μl of bead wash buffer and pellet the beads by centrifuging for two minutes at 3000×g.
12. Immediately remove the supernatant and resuspend the bead pellet in 200 μl of bead wash buffer. <10 μl of Bead Wash Buffer can be left to preserve the bead pellet.

SAFE STOPPING POINT. Beads can be stored at 4° C. (e.g., for up to three days).

### 5.1.5 Second Strand Synthesis

1. Set one heat block to 95° C. and another heat block to 37° C.
2. Thaw the following reagents at room temperature and then keep on ice: RT/SS buffer; dNTP; SS primer
3. Keep SS enzyme on ice.
4. Gently pipette-mix the beads from the previous step five times.
5. Incubate the beads at 95° C. for five minutes.
6. Prepare the second strand mix at room temperature.

| Component | 1 reaction (μl) | Plus 5% overage (μl) |
|---|---|---|
| RT/SS buffer | 40 | 42 |
| dNTP | 20 | 21 |
| SS primer | 2 | 2.1 |
| SS enzyme | 5 | 5.25 |
| Nuclease-free water | 133 | 139.7 |
| Total | 200 | 210 |

7. After five minutes of incubation at 95° C., immediately spin the beads down for 30 seconds at 3000×g and carefully remove the supernatant. Remove the supernatant immediately after centrifugation. If processing multiple samples, leave samples at 95° C. until you are ready to process them (e.g., for up to 10 additional minutes).
8. Immediately resuspend the beads in 200 μl of second strand mix.
9. Incubate at 37° C. for one hour.
10. Add 200 μl of bead wash buffer.
11. Spin the beads down for two minutes at 3000×g and immediately remove the supernatant.
12. Resuspend the beads in 200 μl of bead wash buffer.
   5.1.6 cDNA Amplification
1. Thaw the following reagents at room temperature and then keep on ice until ready for use: cDNA amplification buffer; cDNA amplification primer mix
2. Keep cDNA amp enzyme on ice until ready for use.
3. Preheat a thermocycler to 98° C. and hold until ready to proceed with amplification. Heat the lid to 105° C.
4. Prepare the cDNA amplification mix following the table below and keep on ice:

| Component | 1 reaction (μl) | Plus 5% overage (μl) |
|---|---|---|
| cDNA amp buffer | 100 | 105 |
| cDNA amp primer mix | 8 | 8.4 |
| cDNA amp enzyme | 4 | 4.2 |
| Nuclease-free water | 88 | 92.4 |
| Total | 200 | 210 |

5. Spin the beads down for two minutes at 3000×g and immediately remove the supernatant.
6. Add 200 μl of the cDNA amplification mix to the beads.
7. Split the cDNA amplification mix and beads into four PCR tubes (50 μl each).
8. Pipette-mix each PCR tube before placing the tubes into the thermal cycler.
9. Immediately run the cDNA amplification program on the pre-heated thermocycler as follows. If starting with a tissue section that does not cover the entire substrate or has low cellular content, additional cycles may be required. For example, if your tissue only covers 50% of the substrate unit, increase the cycle number by 1-2. Add 2-3 cycles for tissues with low RNA abundance:

| | | |
|---|---|---|
| Temperature | | |
| ramp rate: 3° C./s | Time | Cycle |

| | | | | |
|---|---|---|---|---|
| 98° | C. | 2 | min | |
| 98° | C. | 20 | sec | 4 cycles (phase 1) |
| 65° | C. | 45 | sec | |
| 72° | C. | 3 | min | |
| 98° | C. | 20 | sec | 9* cycles (phase 2) |
| 67° | C. | 20 | sec | |
| 72° | C. | 3 | min | |
| 72° | C. | 5 | min | |

| | | |
|---|---|---|
| 4° | C. | Hold |

| Fraction of tile covered by tissue | Recommended # of phase 2 cycles |
|---|---|
| >⅔ but not completely covered | 10-11 |
| ⅔ | 11-12 |
| ⅓ | 12-13 |

### 5.1.7 Purification and Quantification

### First 0.6× Bead Purification

1. Prepare fresh 80% ethyl alcohol.
2. Combine reactions from the four PCR tubes into a single 1.5 mL tube.
3. Vortex the purification reagent for 30 seconds and carefully add 120 μl to the tube (0.6× volume of amplification volume). Pipette mix.
4. Vortex to mix for 10-15 seconds.
5. Incubate at room temperature for five minutes.
6. Briefly centrifuge the tubes and place the tubes on the magnetic rack. Once the solution is clear, carefully aspirate and discard the supernatant.
7. Keeping the tube on the magnetic stand, add 500 μl of 80% ethyl alcohol.
8. Wait 30 seconds and remove the supernatant.
9. Add 500 μl 80% ethyl alcohol.
10. Wait 30 seconds and remove the supernatant.
11. Briefly spin the tube to collect the remaining ethyl alcohol at the bottom of the tube.
12. Place the tube back on the magnetic rack and remove the remaining ethyl alcohol carefully.
13. Let the purification reagent dry at room temperature until the beads appear matte (˜ 2 minutes).
14. Remove the tube from the magnetic rack and add 50 μl of nuclease-free water to the tube and pipette the beads to mix well.
15. Incubate at room temperature for one minute.
16. Place the tube back on the magnetic rack.
17. Once the solution is clear, transfer the supernatant to a new 0.2 mL PCR tube. Discard the used beads.

### Second 0.6× Bead Purification

18. Vortex the purification reagent for 30 seconds and carefully add 30 μl to the tube (0.6× volume of amplification volume). Pipette mix.
19. Vortex to mix for 10-15 seconds.
20. Incubate at room temperature for five minutes.
21. Briefly centrifuge the tubes and place the tubes on the magnetic rack. Once the solution is clear, carefully aspirate and discard the supernatant.
22. Keeping the tube on the magnetic stand, add 200 μl of 80% ethyl alcohol.
23. Wait 30 seconds and remove the supernatant.
24. Add 200 μl of 80% ethyl alcohol.
25. Wait 30 seconds and remove the supernatant.
26. Briefly spin the tube to collect the remaining ethyl alcohol at the bottom of the tube.
27. Place the tube back on the magnetic rack and remove the remaining ethyl alcohol.
28. Let the purification reagent dry at room temperature until the beads appear matte (˜2 minutes).
29. Remove the tube from the magnetic rack and add 20 μl of nuclease-free water to elute. Pipette the beads to mix well and incubate at room temperature for one minute. If the sample is expected to have low cDNA yield, elute with 10 μl of nuclease-free water.
30. Place the tubes on a magnetic rack and incubate for one minute.
31. Transfer the supernatant to a new 1.5 ml or 0.2 ml PCR tube.
32. Quantify the cDNA products Concentrations in the range of 0.2 ng/μl and above are acceptable. There should be no significant amount of primer dimer present. cDNA samples can be stored at −20 C for one week before proceeding to the next step.

### 5.1.8 Tagmentation

1. Preheat a thermocycler to 55° C. and heat the lid to 105° C.
2. Thaw the following reagents at room temperature and then keep on ice: Dual Indexing Primers; Tagment DNA (TD) buffer
3. Keep amplicon tagment mix and PCR master mix on ice.
4. In a 0.2 ml PCR tube, add 600 μg of cDNA. Top off to a total volume of 5 μl with nuclease-free water.
5. Add 10 μl of the TD buffer.
6. Add 5 μl of amplicon tagment mix. Pipette to mix.
7. Briefly centrifuge the tube.
8. Incubate at 55° C. for five minutes.
9. After five minutes, immediately add 5 μl of tagmentation Buffer. Mix by pipetting ˜5 times and spin down.
10. Incubate at room temperature for five minutes
11. Add 15 μl of PCR Mix to each tube.
12. Add index primers to the tube.
13. Pipette to mix and briefly centrifuge the tube.
14. Run indexing PCR

| | | |
|---|---|---|
| Temperature | Time | Cycle |

| | | | | |
|---|---|---|---|---|
| 72° | C. | 3 | min | |
| 95° | C. | 30 | s | |
| 95° | C. | 10 | s | 12 cycles |
| 55° | C. | 30 | s | |
| 72° | C. | 30 | s | |
| 72° | C. | 5 | min | |

| | | |
|---|---|---|
| 4° | C. | Hold |

### 5.1.9 Library Cleanup and Quantification

1. Prepare fresh 80% ethyl alcohol.
2. Vortex the cleanup reagent at high speed for 30 seconds. The beads should appear homogeneous and uniform in color.
3. Perform 0.6× cleanup by carefully adding 30 μl of cleanup reagent to 50 μl of the total sample. Pipette mix.
4. Vortex to mix for 10-15 seconds.
5. Incubate at room temperature for five minutes.
6. Briefly centrifuge the tubes and place the tubes on the magnetic rack. Once the solution is clear, carefully aspirate and discard the supernatant.
7. Keeping the tube on the magnetic stand, add 200 μl of 80% ethyl alcohol.
8. Wait 30 seconds and remove the supernatant.
9. Add 200 μl of 80% ethyl alcohol.
10. Wait 30 seconds and remove the supernatant.
11. Briefly spin the tube to collect the remaining ethyl alcohol at the bottom of the tube.
12. Place the tube back on the magnetic rack and remove the remaining ethyl alcohol.
13. Let the cleanup reagent dry at room temperature until the beads appear matte (˜2 minutes).
14. Remove the tube from the magnetic rack and add 10 μl of TE to elute. Pipette beads to mix well and incubate at room temperature for one minute.
15. Place tubes on a magnetic rack and incubate for one minute.
16. Transfer supernatant to a new 1.5 mL tube.
17. Quantify the cDNA products Concentrations above 1 ng/μl are acceptable.
18. (OPTIONAL) If the final library contains an adaptor dimer peak (˜120 bp) that is greater than 2% of the total library, it is strongly recommended to perform an additional 0.8× cleanup,

SAFE STOPPING POINT. Libraries can be stored −20° C. before proceeding to the next step or for long-term storage.

### 5.1.10 Sequencing

Sequencing can be performed with shallow sequencing or deep sequencing protocols. Read lengths can be 50 bp for Read1, 8 bp for Index1, 8 bp for Index2, 50 bp for Read2.

### 5.1.11 Bioinformatics

Bioinformatics methods described can be used to generate spatial maps of targets from sequencing data performed according to Section 5.1.10.

### 5.2 Exemplary Workflow 2

Details of a second exemplary workflow are as follows:

### 5.2.1 Reagent and Apparatus Preparation

1. Thaw the following reagents at room temperature and then keep on ice: RT/SS Buffer (If precipitate is observed, heat the RT/SS buffer at 37° C. for 5 minutes and briefly vortex before use); dNTP.
2. Keep RNase Inhibitor and RT Enzyme on ice.
3. Place the reaction chamber adapter into the thermal cycler and set the thermal cycler to 52° C., keeping the lid completely open.
   5.2.2 Tissue Sectioning and Hybridization with Functionalized Particles of a Substrate
1. Equilibrate the fresh frozen tissue and OCT block (e.g., as an example of smear-prevention layer 15) to −18° C. in a cryostat for at least 20 minutes prior to sectioning. The optimal temperature for sectioning may vary depending on the tissue type.
2. Prepare the hybridization reaction mix in a 1.5 ml tube following the table below and keep at room temperature:

| Component | 1 reaction (μL) | Plus 5% overage (μL) |
|---|---|---|
| Hyb Buffer | 380 | 399 |
| RNAse Inhibitor | 20 | 21 |
| Total | 400 | 420 |

3. Mount the tissue block and the OCT block onto cutting blocks with OCT compound.
4. Section a 10 μm section of tissue.
5. Melt the section onto the substrate in one of the two ways described below:
6a. OPTION 1: For precise placement of the region of interest. Place the substrate unit in the cryostat to chill for 1 minute. Place the chilled substrate unit on the cutting stage and arrange the tissue section on top of the substrate unit using a brush. Make sure that the region of interest is positioned directly over the substrate unit. With the substrate unit and tissue section facing up, melt the tissue section onto the substrate unit by moving the substrate unit off the cryostat stage, and GENTLY placing a finger on the bottom of the slide glass, as shown in the example below. To avoid curling of the tissue, start from one side and slowly move a finger across the region rather than warming it from the center. A small brush can be used to hold the other end of the tissue flat during the initial melting from one end. Do not lift the edges of the substrate unit off the adhesive while melting the tissue. Apply gentle pressure to the back of the substrate unit as long as the substrate unit does not lift off the adhesive.
6b. OPTION 2: For quick placement of the region of interest. Hold a room temperature substrate unit in the substrate unit holder with the substrate unit facing down. Hover the substrate unit over the region of interest. Keeping the substrate unit horizontal, gently lower the substrate unit to bring it into contact with the tissue section. The tissue section should melt onto the substrate unit immediately.
7. Place the substrate unit with the melted tissue section back into the cryostat.
8. Section a 30 μm section of the OCT block.
9. Move the OCT block section onto the substrate unit so that it is covering the entire substrate unit. Alternatively, briefly warm the substrate unit with the tissue section by placing a finger underneath the substrate unit for a few seconds, and melt the OCT section onto the substrate unit with the stamping method described in step 6b.
10. Melt the OCT section onto the substrate unit by placing a finger under the substrate unit and moving it across the substrate unit until the entire OCT section is melted.

SAFE STOPPING POINT. Substrate units can be stored at −80° C. for up to 4 days in a sealed container. To thaw the substrate unit, remove it from −80° C., warm up the substrate unit to room temperature by placing a finger under the substrate unit and ensure the tissue section has re-melted onto the substrate unit before proceeding to step 13.
11. Write the sample name and substrate unit ID on the side of the reaction chamber.
12. Add 400 μl of Hybridization Reaction Mix to chamber 1.
13. Carefully remove the substrate unit from the blue adhesive with tweezers or forceps and place it in chamber 1 containing 400 μl of Hybridization Reaction Mix. Place the substrate unit straight up and down in the middle of chamber 1. There should be no resistance while placing the substrate unit in the chamber. If you encounter resistance, lightly tap the edge of the substrate unit with the tweezer. Make sure the substrate unit is completely submerged. Do not force the substrate unit into the chamber. Correct placement should allow the substrate unit to smoothly slide into the chamber. Tap gently or reposition the substrate unit if it encounters resistance.
14. Seal the chambers with a chamber seal with a straight flat edge.
15. Incubate for 30 minutes at room temperature.
16. Remove the remaining block of tissue from the cryostat and store it at −80° C.
17. Cover any exposed tissue with a drop of OCT compound prior to storage, to prevent desiccation of the tissue sample.

### 5.2.3 Reverse Transcription

1. Prepare the 1× RT wash buffer following the table below for washing the substrate unit prior to RT and keep at room temperature:

| Component | 1 Reaction (μL) | Plus 5% overage (μL) |
|---|---|---|
| RT/SS Buffer | 80 | 84 |
| Nuclease-free Water | 320 | 336 |
| Total | 400 | 420 |

2. Prepare the RT reaction mix in a 1.5 mL tube following the table below and keep on ice:

| Component | 1 Reaction (μL) | Plus 5% overage (μL) |
|---|---|---|
| RT/SS Buffer | 80 | 84 |
| dNTP | 40 | 42 |
| RNase Inhibitor | 10 | 10.5 |
| RT Enzyme | 20 | 21 |
| Nuclease-Free Water | 250 | 262.5 |
| Total | 400 | 420 |

3. Carefully remove the chamber seal from the reaction chambers.
4. Add 400 μl of 1× RT Wash Buffer to chamber 2.
5. Add 400 μl of RT Reaction Mix to chamber 3.
6. Using a pair of clean tweezers or forceps, remove the substrate unit from the Hybridization Reaction Mix in chamber 1 and dip it in the 1× RT Wash Buffer in chamber 2 for 5 seconds.
7. Transfer the substrate unit to chamber 3 with 400 μl of RT Reaction Mix. Make sure the substrate unit is completely submerged.
8. Remove the liquid from chambers 1 and 2 and discard.
9. Seal the reaction chambers with a chamber seal with a straight flat edge.
10. Incubate at room temperature for 10 minutes.
11. Place the reaction chambers onto the reaction chamber adapter in the thermal cycler that was preheated to 52° C.
12. Incubate for 30 minutes with the thermal cycler lid open.

### 5.2.4 Tissue Clearing and Particle Resuspension

1. If precipitate is observed, heat the tissue clearing (TC) buffer at 37° C. for five minutes and briefly vortex before use.
2. Make the tissue clearing reaction mix in a 1.5 mL tube following the table below and keep at room temperature:

| Component | 1 Reaction (μL) | Plus 5% overage (μL) |
|---|---|---|
| TC Buffer | 392 | 411.6 |
| TC Enzyme | 8 | 8.4 |
| total | 400 | 420 |

3. Take the reaction chambers out of the thermal cycler.
4. Set the thermal cycler to 37° C.
5. Carefully remove the chamber seal from the reaction chambers.
6. Add 400 μl of TC Clearing Reaction Mix into chamber 4.
7. Move the tile from chamber 3 to chamber 4.
8. Remove the liquid from chamber 3 and discard.
9. Seal the reaction chambers with a chamber seal with a straight flat edge.
10. Place the reaction chambers back onto the reaction chamber adapter in the thermal cycler that is set at 37° C.
11. Incubate for 30 minutes with the thermal cycler lid open.
12. After the incubation, carefully remove the chamber seal.
13. Transfer the substrate unit to a 5 ml Lo-bind tube.
14. Carefully transfer the liquid from chamber 4 to the 5 ml Lo-bind tube.
15. Rinse chamber 4 with 400 μl of Bead Wash Buffer and transfer the contents to the 5 ml tube containing the substrate unit.
16. Add an additional 500 μl of Bead Wash Buffer to the 5 ml tube containing the substrate unit.
17. Dissociate beads from the glass slide by pipetting the wash buffer mixture directly onto the beads. Be careful not to create excess bubbles as it will make it difficult to see the substrate unit during bead dissociation. It is important to completely dissociate the beads from the glass slide for the region of the substrate that was covered by the tissue section. TIP: Aim the pipette tip at the bead patch when dissociating the beads from the glass slide. Occasionally the residual tissue and beads may still be loosely associated after detachment from the glass slide. Pipet up and down until the cloud of tissue and beads are fully dissociated. TIP: Areas of the substrate unit that are not covered by tissue may be harder to dissociate. For areas that are harder to dissociate, mechanically dislodge the beads from the substrate unit by gently brushing the tip sideways while the tip is parallel to the tile and pipetting across the tile where beads remain. Incomplete dissociation of beads not covered by tissue will NOT affect data quality and performance.
18. Remove remaining glass of the substrate unit from the tube with tweezers or forceps and discard.
19. Transfer the bead suspension to a new 1.5 mL tube.
20. Pellet the beads for 3 mins at 3000×g. A white bead pellet should be visible to the eye.
21. Remove any bubbles from the top of the supernatant.
22. Carefully remove the supernatant without disturbing the bead pellet.

Remove the supernatant immediately after centrifugation as the pellet may slide to the bottom of the tube after some time and become harder to visualize. When removing the supernatant, angle the tip away from the pellet and aspirate slowly to not disturb the pellet. Take care to prevent bead loss while removing the supernatant. If the pellet begins to slide, spin for an additional 30 seconds at 3000×g.
23. Resuspend the bead pellet 800 μl of Bead Wash Buffer and pellet the beads by centrifuging for 3 mins at 3000×g.
24. Immediately remove and discard the supernatant. <10 μl of Bead Wash Buffer can be left to preserve the bead pellet.
25. Resuspend the bead pellet 400 μl of Bead Wash Buffer.

SAFE STOPPING POINT. Beads can be stored at 4° C. (e.g., for up to 3 days).

### 5.2.5 Second Strand Synthesis

1. Set one heat block to 95° C. and another heat block to 37° C.
2. Thaw the following reagents at room temperature and then keep on ice: RT/SS buffer (reverse transcription buffer); dNTP; SS primer
3. Keep SS enzyme on ice.
4. Gently pipette-mix the beads from the previous step five times.
5. Incubate the beads at 95° C. for five minutes.
6. Prepare the second strand mix in a 1.5 mL tube at room temperature.

| Component | 1 Reaction (μL) | Plus 5% overage (μL) |
|---|---|---|
| RT/SS Buffer | 80 | 84 |
| dNTP | 40 | 42 |
| SS Primer | 4 | 4.2 |
| SS Enzyme | 10 | 10.5 |
| Nuclease-free water | 266 | 279.3 |
| Total | 400 | 420 |

7. After five minutes of incubation at 95° C., immediately spin the beads down for 30 seconds at 3000×g and carefully remove the supernatant. Remove the supernatant immediately after centrifugation. If processing multiple samples, leave samples at 95° C. until you are ready to process them (e.g., for up to 10 additional minutes).
8. Immediately resuspend the beads in 400 μl of second strand mix.
9. Incubate at 37° C. for one hour.
10. Add 400 μl of bead wash buffer.
11. Spin the beads down for 3 minutes at 3000×g and immediately remove the supernatant.
12. Resuspend the beads in 400 μl of bead wash buffer.
   5.2.6 cDNA Amplification
1. Thaw the following reagents at room temperature and then keep on ice until ready for use: cDNA amp buffer; cDNA amp primer mix
2. Keep cDNA amp enzyme on ice until ready for use.
3. Preheat a thermocycler to 98° C. and hold until ready to proceed with amplification. Heat the lid to 105° C.
4. Prepare the cDNA amplification mix in a 1.5 mL tube following the table below and keep on ice:

| Component | 1 Reaction (μL) | Plus 5% overage (μL) |
|---|---|---|
| cDNA Amp Buffer | 200 | 210 |
| cDNA Amp Primer Mix | 16 | 16.8 |
| cDNA Amp Enzyme | 8 | 8.4 |
| Nuclease-free water | 176 | 184.8 |
| Total | 400 | 420 |

5. Spin the beads down for two minutes at 3000×g and immediately remove the supernatant.
6. Add 400 μl of the cDNA amplification mix to the beads.
7. Split reaction mix into 8 PCR tubes (50 μl each).
8. Pipette-mix each PCR tube before placing the tubes into the thermal cycler.
9. Immediately run the cDNA amplification program on the pre-heated thermocycler as follows. If starting with a tissue section that does not cover the entire substrate or has low cellular content, additional cycles may be required. For example, if your tissue only covers 50% of the substrate unit, increase the cycle number by 1-2. Add 2-3 cycles for tissues with low RNA abundance:

| | | |
|---|---|---|
| Temperature | | |
| ramp rate: 3° C./s | Time | Cycle |

| | | | | |
|---|---|---|---|---|
| 98° | C. | 2 | min | |
| 98° | C. | 20 | sec | 4 cycles (phase 1) |
| 65° | C. | 45 | sec | |
| 72° | C. | 3 | min | |
| 98° | C. | 20 | sec | 9* cycles (phase 2) |
| 67° | C. | 20 | sec | |
| 72° | C. | 3 | min | |
| 72° | C. | 5 | min | |

| | | |
|---|---|---|
| 4° | C. | Hold |

| Fraction of substrate unit | Recommended # of |
|---|---|
| covered by tissue | phase 2 cycles |
| >⅔ but not completely covered | 10-11 |
| ⅔ | 11-12 |
| ⅓ | 12-13 |

### 5.2.7 Purification and Quantification

### First 0.6× Bead Purification

1. Prepare fresh 80% ethyl alcohol.
2. Vortex the purification beads at high speed for 30 seconds. The beads should appear homogeneous and uniform in color.
3. Remove the PCR tubes from the thermal cycler.
4. Combine reactions from the eight PCR tubes into a single 1.5 mL tube.
5. Measure the total volume of the combined reaction mixture.
6. Calculate the volume of purification beads needed by multiplying the total reaction mixture volume by 0.6. For example, if the total volume is 400 μl, you will need 240 μl of purification beads.
7. Add the volume of purification beads calculated in step 6 to the tube of amplified cDNA.
8. Vortex to mix for 10-15 seconds.
9. Incubate at room temperature for 5 minutes.
10. Briefly centrifuge the tube.
11. Place the tube on the magnetic rack. Once the solution is clear, carefully aspirate, and discard the supernatant.
12. Keeping the tube on the magnetic stand, add 500 μl of 80% ethanol.
13. Wait 30 seconds and remove the supernatant.
14. Add 500 μl of 80% ethanol.
15. Wait 30 seconds and remove the supernatant.
16. Briefly spin the tube to collect the remaining ethanol at the bottom of the tube.
17. Place the tube back on the magnetic rack and remove the remaining ethanol.
18. Let the purification beads dry at room temperature until the beads appear matte (˜ 5 minutes).
19. Remove the tube from the magnetic rack and add 50 μl of nuclease free water to the tube and pipette the beads to mix well.
20. Incubate at room temperature for 1 minute.
21. Place the tube back on the magnetic rack.
22. Once the solution is clear, transfer the supernatant to a new 0.2 mL PCR tube. Discard the used beads.

### Second 0.6× Bead Purification

23. Vortex the SPRI beads and add 30 μl to the tube (0.6× volume of amplification volume).
24. Vortex to mix for 10-15 seconds.
25. Incubate at room temperature for 5 minutes.
26. Briefly centrifuge the tube
27. Place the tube on the magnetic rack. Once the solution is clear, carefully aspirate, and discard the supernatant.
28. Keeping the tube on the magnetic stand, add 200 μl of 80% ethanol.
29. Wait 30 seconds and remove the supernatant.
30. Add 200 μl of 80% ethanol.
31. Wait 30 seconds and remove the supernatant.
32. Briefly spin the tube to collect the remaining ethanol at the bottom of the tube.
33. Place the tube back on the magnetic rack and remove the remaining ethanol.
34. Let the purification beads dry at room temperature until the beads appear matte (˜2 minutes).
35. Remove the tube from the magnetic rack and add 20 μl of nuclease free water to the tube to elute. Pipette the beads to mix well and incubate at room temperature for 1 minute.
36. Place the tube on a magnetic rack and incubate for 1 minute.
37. Transfer the supernatant to a new 0.2 ml PCR tube.
38. Quantify the cDNA products. Concentrations in the range of 1 ng/μl and above are acceptable. There should be no significant amount of primer dimer present.

SAFE STOPPING POINT. cDNA samples can be stored −20° C. (e.g., for one week before proceeding to the next step).

### 5.2.8 Tagmentation

1. Preheat a thermocycler to 55° C. and heat the lid to 105° C.
2. Thaw the following reagents at room temperature and then keep on ice: Dual Indexing Primers; TD tagmentation buffer
3. Keep amplicon tagment mix and PCR master mix on ice.
4. In a 1.5 mL tube, add 4.8 ng of cDNA. Top off to a total volume of 40 μl with nuclease-free water.
5. Add 80 μL of TD buffer
6. Add 40 μL of amplicon tagment mix
7. Pipette to mix
8. Split the reaction mix into 8 new PCR tubes.
9. Briefly centrifuge the tubes.
11. Incubate at 55° C. for 5 minutes.
12. After 5 minutes of incubation, immediately add 5 μl of Neutralization Tagment Buffer (NT). Mix by pipette-mixing and spin down.
13. Incubate at room temperature for 5 minutes.
14. Add 15 μl of PCR Mix to each tube.
15. Add index primers to each tube. Use the same index primers for each of the 8 partitions from the same sample. WARNING: ensure that each sample that will be sequenced together has a unique combination of F and R primers. One F and R primer set will be sufficient for the 8 partitions of a single tile.
16. Pipette to mix and briefly centrifuge.
17. Run indexing PCR.

| | | |
|---|---|---|
| Temperature | Time | Cycle |

| | | | | |
|---|---|---|---|---|
| 72° | C. | 3 | min | |
| 95° | C. | 30 | s | |
| 95° | C. | 10 | s | 10 cycles |
| 55° | C. | 30 | s | |
| 72° | C. | 30 | s | |
| 72° | C. | 5 | min | |

| | | |
|---|---|---|
| 4° | C. | Hold |

### 5.2.9 Library Cleanup and Quantification

### First 0.6× Bead Purification

1. Prepare fresh 80% ethyl alcohol.
2. Vortex the cleanup reagent at high speed for 30 seconds. The beads should appear homogeneous and uniform in color.
3. Remove the PCR tubes from the thermal cycler.
4. Combine the reaction mixture into one new 1.5 mL tube.
5. Measure the total volume of the combined reaction mixture.
6. Calculate the volume of cleanup beads needed by multiplying the total reaction mixture volume by 0.6.
7. Add the volume of cleanup beads calculated in step 6 to the tube of amplified cDNA. Pipette Mix.
8. Vortex to mix for 10-15 seconds.
9. Incubate at room temperature for 5 minutes.
10. Briefly centrifuge the tube.
11. Place the tube on the magnetic rack. Once the solution is clear, carefully aspirate, and discard the supernatant.
12. Keeping the tube on the magnetic stand, add 500 μl of 80% ethanol.
13. Wait 30 seconds and remove the supernatant.
14. Add 500 μl of 80% ethanol.
15. Wait 30 seconds and remove the supernatant.
16. Briefly spin the tube to collect the remaining ethanol at the bottom of the tube.
17. Place the tube back on the magnetic rack and remove the remaining ethanol.
18. Let the cleanup beads dry at room temperature until the beads appear matte (˜ 5 minutes).
19. Remove the tube from the magnetic rack and add 10 μl of TE to the tube and pipette the beads to mix well.
20. Place the tubes on a magnetic rack and incubate for 1 minute.
21. Transfer the supernatant to a new 0.2 ml PCR tube.
22. Quantify the cDNA products. Concentrations should be >1 ng/μl.
23. (OPTIONAL) If the final library contains an adapter dimer peak (˜120 bp) that is greater than 1% of the total library, it is strongly recommended to perform an additional 0.8× cleanup.

SAFE STOPPING POINT. Libraries can be stored −20° C. before proceeding to the next step or for long term storage.

Examples of the method 100 can, however, be performed with additional steps, omission of steps, and/or combinations of steps described.

### 5.2.10 Sequencing

Sequencing can be performed with shallow sequencing or deep sequencing protocols. Read lengths can be 50 bp for Read1, 8 bp for Index 1, 8 bp for Index2, 50 bp for Read2.

### 5.2.11 Bioinformatics

Bioinformatics methods described can be used to generate spatial maps of targets from sequencing data performed according to Section 5.2.10.

### 6. Computer Systems

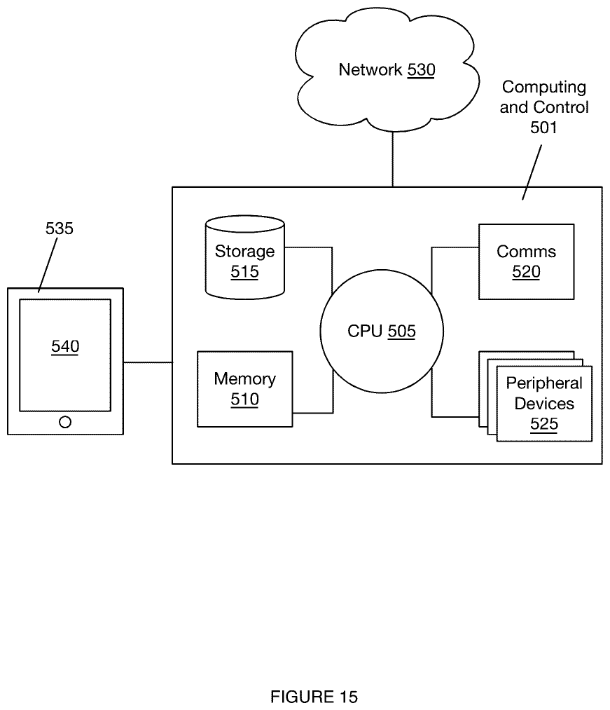

The present disclosure provides computer systems that are programmed to implement methods of the disclosure. FIG. 15 shows a computer system 501 that is programmed or otherwise configured to, for example, perform steps of methods for generating spatial maps of a distribution of targets of a sample, by one or more processes described.

The computer system 501 can regulate various aspects of analysis, calculation, and generation of the present disclosure, such as, for example, generating a spatial map of a distribution of targets of a sample by a set of processes, wherein the set of processes includes one or more of: receiving the sample at a substrate comprising a distribution of functionalized particles, each of the distribution of functionalized particles comprising a stochastic barcode sequence paired with a position on the substrate, promoting interactions between the distribution of targets of the sample, applying a set of reactions to the sample at the substrate, obtaining a set of sequences of a population of molecules generated from the set of reactions, the set of sequences associated with the distribution of targets labeled using the stochastic barcode sequences of the distribution of functionalized particles, and returning a set of positions of the distribution of targets upon processing the set of sequences. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet.

In some embodiments, the network 530 is a telecommunication and/or data network. The network 530 can include one or more computer servers, which can enable distributed computing, such as cloud computing. For example, one or more computer servers may enable cloud computing over the network 530 (“the cloud”) to perform various aspects of analysis, calculation, and generation of the present disclosure, such as, for example, generating a plurality of droplets within a collecting container at a predetermined rate or variation in polydispersity. Such cloud computing may be provided by cloud computing platforms such as, for example, Amazon Web Services (AWS), Microsoft Azure, Google Cloud Platform, and IBM cloud. In some embodiments, the network 530, with the aid of the computer system 501, can implement a peer-to-peer network, which may enable devices coupled to the computer system to behave as a client or a server.

The CPU 505 may comprise one or more computer processors and/or one or more graphics processing units (GPUs). The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some embodiments, the circuit is an application specific integrated circuit (ASIC).

The storage unit 515 can store files, such as drivers, libraries and saved programs. The storage unit 515 can store user data, e.g., user preferences and user programs. In some embodiments, the computer system 501 can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some embodiments, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Embodiments of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, a visual display indicative of generating a spatial map of a distribution of targets of a sample by a set of processes, wherein the set of processes includes one or more of: receiving the sample at a substrate comprising a distribution of functionalized particles, each of the distribution of functionalized particles comprising a stochastic barcode sequence paired with a position on the substrate, promoting interactions between the distribution of targets of the sample, applying a set of reactions to the sample at the substrate, obtaining a set of sequences of a population of molecules generated from the set of reactions, the set of sequences associated with the distribution of targets labeled using the stochastic barcode sequences of the distribution of functionalized particles, and returning a set of positions of the distribution of targets upon processing the set of sequences. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, generate one or more spatial maps with performance characteristics described.

### 7. Conclusions

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or, if applicable, portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts, or combinations of special purpose hardware and computer instructions.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. A system comprising:
a substrate comprising a distribution of functionalized features,
wherein the distribution of functionalized features comprises a set of barcode sequences corresponding to a set of positions on the substrate, and
wherein the distribution of functionalized features comprises molecules functionalized to interact with a set of targets of a sample; and
a smear-prevention layer configured to be positioned over the sample when the sample is positioned on the distribution of functionalized features.

2. The system of claim 1, wherein the smear-prevention layer comprises optimum cutting temperature (OCT) compound.

3. The system of claim 1, wherein the smear-prevention layer comprises OCT compound combined with a sample processing reagent.

4. The system of claim 1, wherein the distribution of functionalized features comprises a set of functionalized particles coupled to the substrate in a random close packed configuration.

5. The system of claim 1, wherein the distribution of functionalized features comprises features with a center-to-center spacing less than 5 micrometers.

6. The system of claim 1, wherein the distribution of functionalized features comprises at least 20 million functionalized features.

7. The system of claim 1, wherein the sample comprises a tissue sample.

8. The system of claim 1, wherein the set of targets comprises mRNA targets of the sample.

9. A method comprising:
generating a spatial map of a distribution of targets of a tissue sample, with a percent reduction in smearing artifacts in the spatial map greater than 60%, upon:
receiving the tissue sample at a substrate comprising a distribution of functionalized features, each of the distribution of functionalized features comprising a barcode sequence paired with a position on the substrate;
applying a smear-prevention layer over the tissue sample at the substrate;
promoting interactions between the distribution of targets of the tissue sample and the distribution of functionalized features;
applying a set of reactions, comprising a hybridization reaction, to the tissue sample at the substrate, with the tissue sample positioned between the smear-prevention layer and the substrate;
obtaining a set of sequences of a population of molecules generated from the set of reactions, the set of sequences associated with the distribution of targets labeled using the barcode sequences of the distribution of functionalized features; and
returning a set of positions of the distribution of targets upon processing the set of sequences.

10. The method of claim 9, wherein the smear-prevention layer comprises optimum cutting temperature (OCT) compound.

11. The method of claim 9, wherein the smear-prevention layer comprises OCT compound combined with a processing reagent.

12. The method of claim 9, wherein the smear-prevention layer comprises a thickness greater than 5 micrometers thick.

13. The method of claim 9, wherein generating the spatial map of the distribution of targets of the tissue sample with the percent reduction in smearing artifacts further comprises:
omitting mapping of data from a first category of features of the distribution of functionalized features, wherein sequences acquired from the first category of features have unique molecular identifier (UMI) counts above a first threshold; and
omitting mapping of data from a second category of features of the distribution of functionalized features, wherein sequences acquired from the second category of features have an associated density greater than a second threshold.

14. The method of claim 13, wherein the first threshold is greater than 70 UMI counts.

15. The method of claim 13, wherein the second threshold is a density greater than 1 particle within an area of 20 micrometers.

16. The method of claim 9, wherein the tissue sample comprises one of a fresh frozen tissue sample and a formalin-fixed and paraffin-embedded (FFPE) tissue sample.

17. The method of claim 9, wherein the distribution of targets comprises mRNA targets.

18. The method of claim 9, wherein the distribution of functionalized features comprises at least 800,000 features with a center-to-center spacing less than 5 micrometers.

19. A system comprising:
a substrate comprising a distribution of functionalized features;
a set of cells positioned at interstitial spaces of the distribution of functionalized features, wherein the distribution of functionalized features comprises a set of barcode sequences corresponding to a set of positions on the substrate, and wherein the distribution of functionalized features comprises molecules functionalized to interact with a set of targets of the set of cells; and
a layer configured to be positioned over the distribution of functionalized features.

20. The system of claim 19, wherein the layer comprises a layer of optimum cutting temperature (OCT) compound.
